(19) United States
(12) Patent Application Publication
(10) Pub. No.: US 2019/0336020 A1
(43) Pub. Date: Nov. 7, 2019

(54) Equipment for monitoring health conditions of monitored persons

(71) Applicant: Vladimir KRANZ, Prague 4 (CZ)

(72) Inventor: Vladimir KRANZ, Prague 4 (CZ)

(21) Appl. No.: 16/086,626

(22) PCT Filed: Feb. 27, 2017
(86) PCT No.: PCT/CZ17/00012

§ 371 Date: Sep. 20, 2018

(30) Foreign Application Priority Data
Feb. 25, 2016 (CZ) PV2016-112
Mar. 11, 2016 (CZ) PV2016-143
Mar. 17, 2016 (CZ) PV2016-157
May 5, 2016 (CZ) PV2016-258
May 25, 2016 (CZ) PV2016-310
Jun. 24, 2016 (CZ) PV2016-374
Nov. 23, 2016 (CZ) PV2016-731
Jan. 24, 2017 (CZ) PV2017-37

(51) Int. Cl. A61B 5/0402; A61B 5/00

(52) U.S. Cl. CPC A61B 5/0402; A61B 5/0006; A61B 5/6831; A61B 2560/0431; A61B 2560/0468; A61B 5/6824; A61B 5/6823

(57) ABSTRACT

Equipment for monitoring of health conditions of monitored persons in different alternatives senses cardiac signals, evaluate it, store it and hand over it further. Monitor is relocateable between auxiliary devices using mounting elements and cooperates with cooperating units.

### TECHNICAL FIELD

This invention relates to a universal monitor of heart signals.

### BACKGROUND ART

The prior art monitors of heart signals allow either a short-time touch of fingers or of another part of the human body to electrodes or the electrodes are fastened for a long time by means of a chest belt or by other means to the human body. Monitors, that would allow a short-time monitoring by touching e.g. of a finger, and if necessary, in case of a longer or permanent measuring, by permanent fastening to chest or to both arms or a wrist for a long-time monitoring, do not exist. Also, in case of using a chest belt for fastening to the chest, the prior art does not offer placement of this belt into a box together with a monitor for easy storage and carrying in case of the possibility to use the monitor by touching a finger or by applying of the monitor, or of a box with the monitor to the chest, without any extracting of the monitor from the box, i.e. instantaneous use of it without any handling to extract the monitor.

### DISCLOSURE OF THE INVENTION

The mentioned drawbacks of the prior art are removed and improvements are implemented by a universal monitor, which is provided with external feeding for sensing of cardiac signals. It is detachably placed on a chest belt. The chest belt is transferrable into a case, where it is conductively linked with contact areas that are off the case and used for sensing of the cardiac signals. Alternatively a universal monitor is inserted into a case with a chest belt, which chest belt is stretched out for sensing of cardiac signals by being grasped by both hands. Alternatively, the sensing is carried out by means of contact areas for lingers of both hands, wherein said areas are connected with areas on said chest belt.

The monitor is transferrable between various sensors of cardiac signals with contact areas and/or it has contact areas placed on itself for short-time sensing of cardiac signals from fingers or from a wrist or for long-time sensing from both wrists and/or from chest, preferably by using of stuck electrodes, and/or by a combination of said methods of sensing.

The controlling and displaying module, into which the monitor transmits the sensed data, makes possible to the monitored person to follow the course of measurement, and to adapt own activities accordingly, for example by actions to minimize arrhythmia or to reach ability to drive a means of transport or to adjust a correct dose of medicaments.

Therefore, some feedback for the above mentioned and other activities is possible. The activities can be modified on the basis of the results obtained on displays, for example on those displays of the control and display modules. Preferably, according to requirements, the data and the results of monitoring are transmitted further into the central surveillance panel where they are processed further automatically, and the results are sent back to the control and display modules, where they are available to the monitored person and/or the results are available to professional healthcare personnel.

Monitor of heart signals is inserted into a case, and it is fastened on a chest belt. The chest belt is provided with contact areas for contact with skin. The contact areas serve preferably as electrodes for heart signals. They are conductive and connected do the monitor. Preferably, this monitor is secured against any dropping-out of the case by backstops.

The chest belt, preferably of some rubber material, inserted into the case by folding it on both sides, and the case is secured against opening up by a small hinged door. Preferably, the door is secured by backstops against opening.

Detachable clips on the electrodes, conductively fastened on the contact surfaces, are connected by short cables to the ECG electrodes, which are placed off the case: They may be touched by fingers of both hands or applied on the chest to carry out short-time monitoring. A part of the case is a hinged door to be tipped back that can be used for a short-time monitoring. Both sides of the chest belt are opened up, they are grasped with both hands or the chest belt is put on chest to provide a better contact than what is that obtained by touching the contact areas by fingers.

The monitor with the chest belt can be extracted from the case. Preferably an elastic belt, preferably extracted from a storage place, is connected to this chest belt. By means of it the monitor is fastened to chest for sensing heart signals by means of contact areas on the case. The monitor processes heart signals and it sends the heart pulses in a data form and/or a ECG curve via a communication unit by means of a wireless link, preferably realized by a Bluetooth, to a local control and display module, preferably represented by a mobile phone, for a detailed display and evaluation, ether live or from memories, or it sends the data directly or via a control and display module to a remote evaluation block. Preferably, the block is represented by a server. Preferably, transmitting is carried out by means of a data network, preferably represented by a WiFi network, or by a mobile operator's network. The module, or the monitor, switch over between the WiFi network and the mobile operator's network automatically, or the monitored person makes it manually, according to network coverage, wherein the WiFi network is preferred because of lower costs, so that there is continuous connectivity with the server. When monitoring the heart activity, preferably, the monitor uses the cost saving communication module, preferably formed by a low energy consuming Bluetooth, and sends heart pulses, preferably continuously, and it sends the heart signals of an ECG curve only on demand issued by the monitored person or by the server, or by a selected participant, or periodically, or when the preset limits of health data are exceeded. The server is accessible for the selected participants, preferably by means of an internet link.

Preferably, at the same time, this communication unit links the monitor also with a watch for operative displaying.

The monitor may be fastened detachably to the chest belt by means of fastening elements, which elements are formed preferably by press fasteners for a long-time sensing of the heart signals from the chest.

Preferably, the contact areas for sensing of the heart signals by touching with fingers or by touching to the chest without any fastening to the chest belt are situated on the monitor.

The monitor may be inserted into a case and snapped into fastening elements, which are preferably formed by press fasteners. The press fasteners are connected with the contact areas that are placed on the case for any short-time measurements of the heart signals by touching them with a fingers, or by touching them to chest. The removed belt is stored under the small door.

The monitor can be connected with a conductively snapped-on plate to the mounting elements, which elements are connected with contact areas for a short-time measurement of the heart signals.

The monitor with the snapped-on plate may be inserted into a case. The case is provided with an opening for making accessible the contact areas for a short-time measurement by touching with fingers of both hands.

Preferably, the monitor is connected by a connector, preferably represented by a USB connector, preferably to ne used for data communication and/or for feeding, from a side to the operative control and display module. Preferably, the module is formed by a mobile phone, for operative evaluation and display of heart signals sensed during a short-time measurement from the contact areas placed on this monitor. Preferably, the monitor is fastened on this operative control and display module by this connector and preferably, for better reinforcing, by a mounting mechanism, preferably formed by a slip-on case spanning this monitor and the module. Preferably, this module is provided with a wristband to be placed on a wrist by means of a fastening mechanism, preferably detachably from this wristband.

The heart signals are sensed from the contact areas or by electrodes, which are present in an optional number. Therefore, the sensing of a one-lead ECG from the contact areas is optionally expandable up to the required number of leads, maximally to 12. Preferably, the wrist of a hand is in contact with a contact area, which is placed on the bottom part of the monitor. Preferably, the next area is placed on the upper side of the monitor for contact of a finger of the other hand. Both contact areas, which are placed alternatively on the upper surface of the monitor, are connected to the front-end ECG monitor for sensing and digitization of heart signals. Alternatively, the heart signals are sensed by electrodes, preferably by such that are stuck on chest and connected by a wire connection to a connector for electrodes on the monitor. In case the monitor is disconnected from the operative module, preferably, the monitor communicates with them by means of a wireless link. The monitor comprises a control unit, a memory, preferably an extractable memory medium, preferably a SD card, for storing of the sensed values of the body of the monitored person in a data form. This allows to monitor the person subject of surveillance on the basis of the actually transferred data, and also subsequently on the basis of data stored in memory. Preferably, the data from the required time period is processed, preferably 24-hours data, preferably into an evaluation of the “Holter” style. Preferably, such monitoring may be carried out for a different period of time than 24 hours. Then the results are re-calculated to the 24-hour basis.

The operative module, which is preferably of smaller size and preferably placed detachably on said wristband for operative displaying on a wrist, preferably communicates by means of wireless link with the control and display module, which is placed loosely, which is of larger dimensions for a detailed displaying. In case of connecting to the module, the monitor does not require any accumulator. In case, it is provided with one, such accumulator may be used as a power source for bridging when the main accumulator in the module is being replaced to avoid interruption of operation. This is done with the help of the fastening mechanism, which is preferably provided with a small door.

The monitor, which is disconnected from the operative control and display module, and which is placed on the chest belt on the chest of the monitored person, preferably communicates via a wireless connection with the operative module and/or with the control and display module, and preferably simultaneously with a wristwatch to be used for sport activities. Preferably, the wireless link is represented by a Bluetooth or an ANT or by an induction connection, preferably at about 5 kHz, and/or preferably, this monitor communicates with the server directly, or preferably via the control and display module of the server, preferably via a data network, preferably represented by a WiFi network or by a mobile operator's network, for evaluation and displaying of numerical data and/or curves, preferably of the pulse rate with the limit curves of the regular pulses, arrhythmia, variability, ECG, which may be evaluated and displayed also in the module. They are displayed individually or simultaneously in optional allocations.

When the monitor is used independently, that is, it is not connected to the module by a connector, and it communicates wirelessly, preferably it is used as a wireless low energy Bluetooth link (BLE). For monitoring of the heart activity, it is enough to transmit only the heart pulse rate value, what consumes less energy than what would consume transmission of the full signal for the one-lead ECG.

Preferably, the limits are set, for example the values of pulse rate or arrhythmia, when an alarm signal is generated. When it is desired by the monitored person and/or automatically, when the limits have been exceeded, it is switched over to temporary ECG transmission to have a possibility to find the reasons why a warning signal was initiated. This modification saves energy of the accumulator, which accumulator then serves for a longer period of time without any charging than as it is in case of an uninterrupted ECG transmission, which ECG is however recorded during the whole period of sensing on a memory medium, which medium is a SD card preferably. From this SD card, the stored data can be replayed when only the data exceeding the preset limit are displayed automatically. Thereby, energy is also saved and the period of time, which is consumed for viewing of the data, is shortened.

Preferably, the monitor is connected to the module via a connector. The connector is provided with a slip-on case for reinforcing. The heart signals are sensed by the sensing elements, preferably by contact areas on the upper part of the monitor by means of fingers of both hands. The monitor is inserted into the slip-on case, which case is mounted on the module cover, preferably by sticking.

Preferably, the monitor is placed on the control element. Preferably, the control element is represented by a steering wheel, or by a handlebar, or by levers, or by a control element of an aircraft or by push-buttons of a machine, or by contact areas of a machine or by a means controlled by the monitored person. Preferably they are formed by a means of transport, preferably they are represented by a vehicle, an aircraft, a train, a motorcycle, a bicycle, a caterpillar vehicle, a tractor, or preferably it is formed by a machine controlled by the monitored person.

This monitor is fastened to the control element firmly or detachably by means of a fastening element. Preferably, this element is represented by press fasteners, or preferably by means of a sleeve, on which sleeve this monitor is fastened firmly or detachably, preferably by means of press fasteners.

Preferably, the monitor is linked by a wireless connection to the control and display module, which is preferably placed detachably in a holder, which is fixed, preferably detachably, on the vehicle, preferably on the windscreen, or on the dashboard of the vehicle, and/or it is connected to a module, which is built in the vehicle, preferably in the panel with the control element and with the display element, preferably formed by the display. Alternatively, the monitor is mounted on the built in module by a wire connection. Preferably, for operative displaying, the monitor is connected simultaneously to the wristwatch and/or to the module on said wristband, preferably one of smaller size, via a wireless connection. Preferably, via a wireless connection, the module is also connected to the evaluation block, preferably via a data network, preferably a WiFi network, and/or via a mobile operator's network. Preferably, the built-in module is linked via a wireless connection to the control and display module in the holder. Said modules, together with the operative module, are adapted for identical displays, preferably on the basis of data from the module, preferably in the holder, or freely deposited, for example in a pocket.

On the control element, preferably on the steering wheel or the sleeve, the sensing elements are placed, wherein, preferably, the contact areas form electrodes for sensing of heart signals from both hands during controlling the means by the monitored person for processing to a value or a pulse rate curve and/or arrhythmia and/or variability and/or ECG. They are connected to the monitor directly or via a fastening element, preferably formed by the press fasteners. Preferably, the results of monitoring are evaluated automatically, or by the health care personnel, on a PC server, or by the remote participants, and the result is sent back to the monitored person as data via a mobile data network, preferably a mobile operator's network, or by phone. Preferably, the fellow travelers are monitored by other monitors connected to the module and further to the evaluation block. Preferably, an emergency call push-button and a reset push-button are placed on the control panel, with which the monitored person may reset it. It may be used in any health or traffic emergency service. Preferably, an accident detector is connected to this module, which accident detector monitors preferably any vehicle impact, vehicle overturn, shooted “air bag” and other phenomena that indicate an accident. Preferably, the accident detector transmits data about such accident via the module to the server, and preferably simultaneously, data from sensors about health state of the monitored person are transmitted, which data are senses by the monitor, or by the multifunction chest belt, preferably by the monitor of heart signals on the chest belt or by the sensors themselves that are sensing physiological values of human body, such as temperature, breath, oxygen content in blood, and other data as shown in other figures. The monitor in the chest belt is connected to the module in the holder or it is built in. Alternatively, the heart signals are sensed by a monitor with contact areas placed on it, which monitor can be moved from the chest belt to a plate, a wristband of the other hand, a case, or it operates independently. Preferably, the health state off limits or an accident initiate a warning signal, preferably resettable one, by means of the reset push-button, which warning signal is transmitted to the server of the surveillance center, preferably, when it is not reset. In case of the permanent connection of the monitored person to the monitor or to the sensor, this person is under constant surveillance, in case of connection for surveillance by the surveillance center in the evaluation block, which center, in case of health data off limits, preferably connects with the monitored person by means of mobile via phone connected instruments or via a hands-free data link provided with a microphone and a speaker. Preferably, they are associated with the module. The system transmits GPS coordinates automatically in case of an accident or in case the health data are off limits, or on demand. Preferably, the monitor is connected with the vehicle computer for coordinated sharing and transmitting of data to the server and to the operation center that monitors vehicle accidents. Preferably, the module is connected to the external GPS antenna of the vehicle, which antenna is preferably placed on the vehicle roof. Preferably, contact areas connected to the monitor are placed on the side of the fellow-traveler for testing of the fellow-traveler.

On the control and display module there is an emergency call push-button, which is pushed to transmit an emergency call to the surveillance center, which is also used to transmit when the set health characteristics are off limits. Preferably, the emergency call is anticipated by a warning signal, which signal allows to decide whether to cancel the emergency call. Having received the emergency signal, the surveillance center is adapted for voice communication with the driver and the center has access to health data detected by the monitor or by detectors in the module of the additional units and on the basis of an evaluation, it is adapted to switch on warning lights in the vehicle, and preferably to stop the vehicle or to carry out long-distance control, for example to park the vehicle on a sustainable place.

A traffic means, preferably a vehicle is provided with video cameras that make the long-distance control easier.

Alternatively, or when the vehicle is not connected with a surveillance center having the ability of long-distance control of the vehicle, preferably, the vehicle is adapted for automatic switching on of the warning lights, and to stop the vehicle or to guide the vehicle to a suitable place where it can stop and park on the basis of GPS and the Google map, when the driver does not reset the warning signal.

Preferably, an alarm is induced also after a period of time has elapsed on a counter, if it is not reset on time.

A wristwatch or a module on a wristband, connected wirelessly on a module and/or on a monitor, are adapted preferably for taking off this wristband for phone operation, which operation is possibly by attaching it to an ear. Preferably, the wristwatch is provided with parts for communication via a mobile operator's network or it is adapted for displaying and implementing.

Preferably, the monitor can be placed on a sleeve and it communicates with the operative control and display module, which is represented preferably by a smaller mobile phone, which is placed preferably on said wristband for operative monitoring and for communicating with the module, which is preferably bigger, for detailed monitoring of an evaluation block. Preferably, the operative module communicates with a module via a wireless connection, and it is adapted for displaying data from the module, preferably about the phone calls, SMS, and it is adapted for taking over the calls. Preferably, the module is adapted to display homothetically the displaying on the display of the module, and vice versa. Preferably, the monitor may be installed in the means of transport firmly, and it may be connected by contacts on the control element. Preferably, the module is replaced by a vehicle computer, via which the means can be controlled preferably, for example to park it in case of an emergency call or when the health characteristics are off limits.

For continuous monitoring also in the time period, when the means is not controlled by both hands, the preferably detachably fastened monitor is removed from the control element or the sleeve and it is fastened to the chest belt, to the wristband on a wrist, which can be connected via a connector to the electrodes by a wire connection with electrodes, preferably stuck to the chest, or it is fastened to the operative module, preferably of smaller size, for mounting on the wristband on a wrist, preferably formed by a mobile phone or watch, for continuous sensing of heart signals by electrodes, preferably stuck to the chest, or for occasional sensing by contact areas. For occasional monitoring, preferably, the monitor can be moved into a case.

The monitor with one contact area on its bottom part, which sits on a wrist of one hand, and with second contact area on its upper part for contact with fingers of the other hand or alternatively, connected with an electrode, preferably stuck on chest as an alternative for contact of the other hand, or connected to an electrode, preferably of the wristband of the other hand by a wire connection.

The monitor can be connected by contact areas to the control and display module via a connector.

Preferably, the monitor can be placed on some clothing, preferably on a tee-shirt, and it is connected by a wire jumper with the contact areas on said clothing, which are situated in the chest area. The monitor may be completed by a second one, which is placed under the waist and is connected by wires with the contact areas, which are placed in the chest area.

The module of additional units for a wider range of the monitor features may be situated in the module, or the monitor and sensors and electrodes are connected to it, which allow sense and process biometric data of human body, which data are then evaluated and displayed in the module or in the evaluation block.

Preferably, the monitor can be mounted on some means operated by human operators, preferably formed by a single track means of transport, by a bicycle or by a motorcycle, and this on the handlebar, firmly or detachably, preferably by means of press fasteners, or on the sleeve firmly or detachably. This monitor may be connected via a wireless connection to the module, which module is mounted by a holder to the steering handlebar firmly or detachably. By connection this module may be connected also with the watch and/or with the evaluation block.

The monitor may be situated on the holder, preferably with a handlebar provided with contact areas that are linked by a wire connection to this monitor, which monitor is mounted on or in the module with a displaying element, and further linked via a wireless connection. The Steering handlebar on the holder is used preferably for sensing of heart signals by touching with both hands.

For sensing of the heart signals, also in case when the monitored person drives a single track vehicle with hands in gloves, the glove is provided with contact areas in the glove, which contact serves for providing contact with the hand, wherein the contacts are connected to the external contact areas of the gloves by a wire connection that provides conductive connection of hands by external contact areas attached to the contact areas of the handlebar or the sleeve of the single track vehicle, when the hands in gloves are attached to them. The contact areas on the handlebar are connected by a short cable to the monitor, or the monitor is placed on a wrist, or on one of the gloves, or on a wristband, and is connected by a wire connection with a contact area in the second glove. They are connected by a wire connection to the monitor.

In one embodiment the monitor is mounted on a said glove or the wristband, and it is connected with the contact area for contact with one hand and it is connected by a wire connection across shoulders with the glove or the wristband with a contact area for contact with the other hand to monitor the heart signals.

In a preferable embodiment the monitor is situated in the control and display module, which is preferably formed by a mobile phone, where heart signals are preferably monitored via contact areas, which are placed on it or by electrodes, which are connected by a wire connection.

The module of additional units may be placed in the monitor or in the control and display module, which module preferably comprises a module of internal sensors provided preferably with body temperature sensors, a blood pressure detector, a sleep phase detector, and a on light based blood oxygenation detector. A set of external sensors, preferably supplementary to the internal sensors, is connected to the module, which sensors are preferably an alcohol level detector, an addictive substance detector, a body temperature sensor, a breath sensor, a blood pressure detector, an ECG detector, an EEG detector, an EMG detector (an electromyograph), a sleep phase detector, a light detector, preferably that of blood oxygenation and pulse rate sensing, preferably supplementary or replacing the module of internal sensors, which is connected to the sensors, which are placed on the mobile phone, which are connected by means of a link and/or a wire via a connector for connecting of detectors and probes. Preferably, the sensors are placed on a tee-shirt for sensing of signals and values from the selected spots on the upper part of the trunk of the monitored person, wherein they are connected by wires or wirelessly to the module and they supplement or replace the sensors in a set of the internal or external sensors and/or they are placed on the chest belt, which is connected by wires or wirelessly to the module. Preferably, the sensors and/or the external ECG electrodes are placed on the steering wheel of the transport or other means with human operators, preferably on the steering wheel of an automobile, wherein their signals are brought to a mobile phone by a wire connection or via a wireless connection. Preferably, the ECG signals are also sensed in this way and transferred further to the ECG detector. Preferably, the module of additional units is placed in a transportation means or in another means, where the driver or the operator has dressed on the chest belt, which is provided with a block of sensors or sensors, which are in a long-distance communication by means of a radio link in a means built in a module, preferably in the module, and via this module or via a data network of a mobile network operator the communication with the server and with the central surveillance panel surveilling the drivers or attendants is implemented. Operation and displaying for an operator or a driver of a means of transport or of another equipment is implemented preferably by a built in module with touch display. Alternatively, the described elements are not built in, but they are separate.

The operative control and display module, preferably of smaller size, can be placed detachably on said wristband for the operative display, preferably formed by a mobile phone or a watch or a monitoring watch or an operative control and display unit.

If it is suitable to use a control and display module, preferably of larger dimensions, for a detailed display, preferably it is represented by a mobile phone, or a tablet, or a PC, or the control and display unit.

Preferably, the evaluation block for evaluation of the sensed and processed data is formed by a server, and/or a PC, and/or a mobile phone, and/or a tablet.

Preferably, data is displayed on a display element, preferably represented by a display, as curves and/or numerical values. The values are in particular the pulse rate value, arrhythmia degree, in degrees or in percents of the arrhythmical pulses from the total number of pulses within the selected time unit, the value of variability in degrees, and the curves of pulse rate, preferably with limits of the regular pulses, the curve of frequency of the occurrence of the arrhythmical and regular pulses, the curve of the ECG variability, wherein, they are displayed simultaneously or subsequently, individually, or in optional allocations.

In case the values get off the adjustable limits, preferably, a warning signal is issued which signal, when it has not been reset by the reset push-button, is changed preferably into an alarm, an alarm is issued instantly, and it is transmitted into the selectable place of destination by phone and/or data link and/or SMS, for example to the server.

Simultaneously with a warning signal the alarm type is displayed, which initiated it. In case of sensing the heart signals in the form of heart rate, preferably, variability, arrhythmia, heartbeat rate, omitted pulses are evaluated.

An alarm is initiated by a too low value of variability, what indicates bad psychic and/or physical state of the monitored person, and requires caution or preventing of driving or controlling of means, or in case of athletes during performing a sport activity. On the displaying element, preferably on a display, in particular for monitoring of a person, preferably instead of the “Variability” the “Condition” is for example displayed, what for laymen better reflects the state, which the value represents. A low value of variability, lower than about 2, also indicates tachycardia or flutter, in particular in case of simultaneously increased pulse rate above the normal value measured at rest. This requires checking of correct occurrence of only one P wave for confirmation of this diagnosis by means of ECG. Also, an alarm is initiated by a too low or a too high heart rate value, which is above the adjustable value or exceeding of the adjustable pulse omission. In case of arrhythmia, in case of arrhythmia increase above the adjustable value, which value indicates atrial fibrillation, which has to be checked by means of the P wave occurrence on an ECG.

On the contrary, when decrease in the usual degree of arrhythmia and simultaneous step decrease of variability are observed, flutter or tachycardia are indicated, what is highlighted by eventual increase of heart rate at rest, and it is necessary to verify the P wave in the ECG to confirm the diagnoses.

Also, an alarm may be initiated by an increased number of extrasystols in comparison to the adjustable value, and it is necessary to check in the ECG, whether there are not any dangerous ventricular extrasystols that are dangerous in particular during physical activity.

Simultaneous monitoring of variability and arrhythmia is important for the heart condition evaluation. The variability curve and the arrhythmia curve provide an overview about the state of the monitored person with regard to the activity carried out within a time course. From said values it is determined whether the monitored person is a roadworthy person to drive the transportation means or fit for an athletic activity and whether the person should be medically examined to avoid any worsening of the health state or to exclude any life threat.

The sensing and evaluation of pulse rate is carried out in the control unit of the monitor automatically and/or in the module and/or in the evaluation block and/or visually by means of subsequent or current displaying of said values and/or curves on the displaying element. Preferably, this is widened by adding evaluation and displaying of the ECG curve, where the atrial and ventricular extrasystols, heart attack indication, and transfer, missing or multiplied P wave and other pathologies are evaluated.

For these diagnoses, simultaneous or subsequent displaying of said values and curves on the displaying element is preferably applied, where preferably, the automatic evaluation is checked from the control unit.

By evaluation of the state, it is confirmed that the monitored person is roadworthy, or able to operate the means, preferably those of transport, and the risk is minimized that an accident will be caused by dangerous conditions of drivers, such as e.g. micro-sleep, heart attack, stroke, heart failure, skipping of heartbeats, when the occurrence of said events is indicated with certain probability, with low value of variability, arrhythmia, by ECG course.

In case of athletes, health risks during performance are decreased by monitoring of said values and curves and conditions for effective training are ensured.

By preventive monitoring, risks following from the specified pathological states are decreased for the monitored persons and their prevention or treatment is supported thereby.

In case of sensing heart signals of a monitored person, which is sitting on a seat in the means, preferably, a shortened chest belt provided with a monitor is used, preferably placed under the clothing, preferably formed by a shirt or a T-shirt, which belt is pressed to chest by a pressure belt, preferably comprising two parts connected by a buckle in the middle, which parts are wound on reels provided with springs, which springs, after extraction and release again, wind said parts of the pressure belt back into the reel. Therefore, preferably, the pressure belt is comprised of two parts, wherein each part is wound on one reel, which reels are placed on both sides of the backrest of the seat of the vehicle, which belt is fastened by a buckle, preferably in the middle between the reels. This arrangement is adapted to allow free movement of the driver. When the driver moves forward or sideways, the belt accordingly unwinds from the reel and again winds on the reel. The springs provide for stretching of the pressure belt to press the chest belt to sense the heart signals. Alternatively, the belt is unwound from one reel and it is inserted by a tongue into a mounting opening in the backrest on the other side and the tongue is locked to prevent pulling it out.

The pressure belt encircles the chest and is wearable also outside the vehicle. Pressing of the chest belt to the chest of the monitored person is provided by its elastic part.

Preferably, the chest belt is inserted under the clothing. Preferably, it is a shirt provided with electrodes and a monitor, which is preferably detachably fastened by press fasteners, with put on extensions provided with U-shaped profiles that hold the pressure belt, which presses the chest belt to the chest in a position above it, so that the pressure belt fits across the clothing into the U-shaped profile.

In a preferable solution, the chest belt with the monitor, which is preferably fastened by press fasteners, is provided with openings for the fitting extensions with U-shaped profiles or magnets. Preferably, when the pressure belt is used, they are placed instead of the elastic belt used for mounting on the chest by spanning. The pressure belt with a magnet is held in the position above the chest belt by magnetic force of the chest belt magnets, which magnets are placed under the clothing.

The pressure belt falls into the extensions with U-shaped profiles so that it holds the monitor in the required position. Preferably, the pressure belt is solved as extendable from the reels, which reels are preferably placed in the backrest of the seat, its halves are connectable by means of a buckle and a tongue, which may be inserted into the buckle, with buckle is provided with a releaseable safety lock against slipping out. Preferably, a chest belt is fastened to the pressure belt, preferably by means of a firmly mounted belt, which is formed preferably by a bridge.

Alternatively, a link to the monitor passes through the pressure belt, wherein the monitor is placed preferably in the backrest, wherefrom it passes a signal on, via a wireless connection or via a wire connection.

Alternatively, the chest belt is adapted for mounting to the pressure belt by means of a bridge, which bridge may be inserted from the outside after the clothing is opened, which clothing is a shirt preferably, whereby pressure of the chest belt to the chest of the monitored person is secured. The pressure belt may be locked by the buckle.

In another embodiment, the chest belt and the pressure belt are provided for fixing of the connection of the chest belt and the pressure belt instead of the U-shaped profiles by magnets, which magnets may be mounted both on the chest belt and the pressure belt, so that they affect themselves through the clothing and maintain the chest belt in the desired position on the chest.

For the purpose of sensing of the heart signals the safety belts of the vehicles may be used as the pressure belt, instead of the usual way which consists in a sash running obliquely across the chest, by means of two pressure belts in the waist area and in the chest area for better safety during the simultaneous pressing of the chest belt to the chest by one of the pressure belts, preferably by the upper one, which serves as the safety belt simultaneously, whereas the second belt, preferably the bottom one, serves only as the safety belt preferably.

A combination of the safety belt, which belt is placed obliquely across chest in usual way, and of a pressure belt which belt is placed horizontally across chest, for pressing of the chest belt, which belt serves also as the safety belt, preferably to increase safety, is preferable

In another embodiment, the safety belt is placed obliquely across chest and serves simultaneously for pressing of the chest belt, which is inserted under the clothing and secured in a required position, preferably by magnets on the chest belt with counterparts, which are placed preferably on the external side of the clothing.

The monitor may be used for a testing equipment of the current health condition, in particular of the heart condition and the psychic condition, in particular for determination of the roadworthiness of a person to drive means of transportation or to control a machine or to carry out some activity prescribed by a job description, which activity required some competence or for a preventive actual health condition check, e.g. before a sport activity, or in the state of nausea.

A testing equipment, which is adapted for multiple testing of a greater number of monitored persons, preferably consists of a stand provided with electrodes, which are formed preferably by contact areas, preferably in the form of a handlebar, on which stand a monitor is placed firmly or removably, which monitor is fed by an accumulator preferably, which accumulator is charged by an accumulator charger.

The output of the monitor, which monitor is mounted preferably on the steering handlebar detachably, is connected by a wire connection and/or via a wireless connection with the controlling and displaying module, which module is placed on the stand and/or to a remotedly placed module for a dispatcher.

The testing equipment is adapted for short tests of values above curves determined and calculated from the heart rate, preferably within one minute, what is suitable also for multiple testing by touching electrodes with both hands when seated, but also when standing, when the monitored person may not be in absolute rest.

Preferably, the initial test tests the values derived from the heart rate and ECG is not tested within the initial test preferably, as for the pulse rate test a more simple, and therefore, cheaper external equipment is sufficient, and also such test is made more quickly, and can be sensed from hands. Preferably, in case the initial test is off limits, the ECG tests are carried out at rest, preferably when lying or at least when sitting, by means of a monitor placed preferably on the chest belt. In this, for the initial determination of the health condition of the monitored person, the evaluation made from pulses is mostly sufficient, and considering the simplicity of the test, and considering the complexity, the ECG test is preferred.

Apart from placing of the monitor and the electrodes on the stand, the testing equipment may be implemented in another preferable embodiment with electrodes that are preferably formed by contact areas and with the monitor placed optionally on other equipment or parts.

The testing equipment for the one-time test may be applied for the individual test, e.g. in a means of transport or on an equipment, which is controlled by the monitored person, or at another suitable place, when the electrodes, which are formed preferably by contact areas that are adapted for a short-time touching with fingers or with a palm of both hands simultaneously, e.g. on the steering wheel, or the sleeve, or the control levers of the equipment, or it is adapted for the one-time test.

For a long-time test, the electrodes, which are connected to the monitor, are adapted for uninterruptible contact with the body to sense the heart signals by being placed e.g. on the chest belt, the wristbands, or by sticking to the chest.

Alternatively, the steering handlebar is placed on a Rotoped, where on the operative and the displaying module, which is built in the Rotoped preferably, the cardiac activity detected by the monitor, which is placed on the Rotoped, is detected by contact areas placed on the handlebar or placed on the chest belt.

Preferably, physiological data of the monitored person pedaling the Rotoped are monitored on the module, for example the calories burnt, rotation speed time, time, preferably on a shared display, with data on the heart activity and/or health data.

The display element of the monitor, which is formed preferably by the display, in the starting phase of the initial test with initial display with a notice giving the instruction to start the test by pressing the push-button “Start”, and by touching the electrodes with both hands, which electrodes are formed by contact areas preferably.

After pushing of the push-button “START” for a preset period of time, which is necessary to stabilize pulse rate curves and others, preferably the displaying element displays a waiting image with a “count down” of the time to starting of the test.

The testing image displayed shows values and/or curves, optionally those of the pulse rate and/or of the ECG and/or of the arrhythmia, and/or of the variability, or optionally the testing image is not displayed, and the waiting image is displayed till the end of the test, and than it is replaced directly by an image with results.

After some adjustable time, preferably a short one, up to 1 minute, to make possible that a great number of monitored persons can be tested within a short period of time, a summary of the test results is displayed, which summary shows good state as OK allowing the person to drive the respective traffic means or to control a machine, what indicates good health state. When the result is unsatisfactory “NO GOOD” (N.G.), certain action is required, e.g. checking of the test result by ECG as described on the displaying element before it is permitted to perform the required activity, e.g. driving of a means of transport.

Preferably, in case of the N.G. state, the test is repeated, preferably for a longer period of time, to exclude errors of the initial test, preferably on another set of the testing equipment. On the display displaying the results the monitored person is prompted to do so by an instruction to repeat the test. In case of a second test, carried out on the same set of the testing equipment, the image with results shows a push-button for making a second test, and the time indicated by the prompt, which has elapsed till the moment the push-button was pushed, is monitored. In case of the NG result of the repeated test, one of the next recommended actions is to carry out an ECG examination, and this on the testing monitor on the stand, if it is provided for it, or on other monitor, preferably on one with electrodes that are for higher accuracy attached on the chest, and preferably equipped by electrodes for up to 12-lead ECG, which ECG is evaluated automatically in the module and/or by a trained person and/or by the health care personnel, preferably by a physician present on the place of the testing equipment, or the heart signals monitored preferably by the monitor, preferably by the module are transmitted by means of a long-distance link, preferably via a Wifi network or via a mobile operator's network into an evaluation block, preferably into an evaluation block preferably of a server and/or to remote participants for evaluation by them. Preferably, this is carried out in the server automatically and the result is transmitted as data back to the display element of the module and/or it is reviewed by professional health care personnel, which personnel communicates with the dispatcher preferably present at the testing place and/or with the monitored person by phone or by sending data.

An image with results of the current test is displayed by pushing the push-button, preferably marked “RESULT” on the displaying element of the module, and actual measured values and curves are displayed. In a long-time testing heart signals are sensed by electrodes permanently applied to the body of the monitored person, e.g. attached to the chest belt with a monitor, and the processed data are displayed, and/or stored in a memory medium, e.g. of the control and display module. In case the result is requested, preferably by a push-button placed in the module, the result of the actual test based on said data is processed and displayed.

Preferably, the test is initiated automatically by putting a hand on the electrodes, which are formed preferably by contact areas e.g. on the steering wheel, what is registered by the detector of heart signals, which detector is preferably comprised in the monitor. This is preferable in case of testing during a ride, because to carry out a test it is not necessary to push the START push-button.

Eventually, the test is initiated preferably by a voice command sensed by a voice decoder provided with a microphone interconnected with the module, what is preferable in particular during a ride or in controlling of a machine.

A detailed initial image, a display element, preferably on the module, which image is preferably displayed during initialization of the preparatory phase to a short-time test of the condition by the respective command entered e.g. to the module, is an image which shows an instruction, e.g. “Enter ID” and “To test health condition press the push-button “START””.

Instead of pressing the push-button start, preferably the test is released automatically after gripping the electrodes, e.g. on the steering wheel, by both hands, what is registered by the detector of heart signals, preferably in the monitor, or by a voice command registered by the voice decoder, what is prompted by an instruction displayed on the initial display. Preferably, the initial display is provided also by a push-button for prolonged test, e.g. that of double length, where e.g. by each knocking on the push-button the test length is doubled, and by holding it the test length is altered to a long time one.

On the waiting display an instruction is displayed, e.g. “Hold the steering handlebar by both hands in the place of electrodes and wait till the test measurement starts”. Further, preferably, the instruction e.g. “Wait, test setting is running” may be displayed here. Preferably, time counting to the test start, carried out by the counter, is displayed here.

After elapsing of the waiting display, the displaying element shows preferably the test course, displaying the course of measurements, preferably with pulse rate curves and/or values with limits of regular pulses, arrhythmia, and/or variability and/or ECG. Preferably, this screen displays also an adjustable delay after pushing of the push-button, after which the waiting display is displayed, to hide the deformed curves during the stabilization of the testing equipment, for example by noise, after touching the electrodes by hand.

After the test lasting preferably up to 1 minute, an image with test results is displayed, e.g. “Test OK”. In the instruction, a notice appears preferably that the tasting was terminated, e.g. “Push the push-button OK if ready for the next test”.

If the result of the test is negative, the notice “NO GOOD” N.G., preferably appears in the image with results as well as the instruction to repeat the test, preferably a longer running test, e.g. up to 3 minutes, to eliminate testing error. In case, the test is again N.G., and in case the ECG has not been tested within the initial repeated test, this screen with image of the repeated test displays apart from the results a recommendation to carry out an evaluation of the ECG test.

Preferably, the ECG is not tested in the initial test, because for the pulse rate test is sufficient the simpler, and therefore cheaper equipment, and this test is shorter and can be sensed from hands, e.g. while standing, when the monitored person does not need to be absolutely at rest, so as it is required for the ECG sensing. At the same time for initial testing of the physical condition of the monitored person, the evaluation from the heart rate is mostly sufficient, and considering the simplicity of the test, and considering the complexity of the ECG test, it is preferred.

Preferably, the ECG test is carried out on another monitor with ECG than what is the one, which has been used for the initial condition test. This makes possible to use a cheaper monitor without any ECG for the initial test, and also in case of measurements of a group the monitor is not blocked so as it would be the case when the monitor is used also for the ECG, which measurement takes a longer time than the pulse rate test.

For an ECG test the monitored person is brought into the maximal possible rest in lying or at least sitting position, and the monitor is connected to electrodes, which are formed preferably by contact areas for sensing of the heart signals from the chest, e.g. on the chest belt or on the electrodes affixed on the chest.

The result of the initial measurement from the pulse rate and/or of a repeated measurement and/or of the ECG measurement is preferably used to send data into the evaluation block, preferably into the server, where they are evaluated automatically, and the result is sent back as data into the control and display module of the monitored person or into the dispatcher's PC or the ECG is evaluated by professional health care personnel, e.g. by a physician, who reports the result by a data report or by phone to the dispatcher, who organizes the testing of a group or to the monitored person.

For an ECG test to be carried out in the testing equipment, its monitor is preferably provided with more electrodes, i.e. up to 10 electrodes, for sensing of up to 12 lead ECG. Alternatively, another ECG equipment is used allowing to send the result into the evaluation block, into a server and/or to remote participants.

Preferably, the results of tests are evaluated and displayed according to a table O displaying on the displaying element, from which table the results are taken over according to the measured values, preferably as numbers and word, in case of a test with negative result. The test result evaluates preferably the pulse rate, arrhythmia, health condition, what is preferably the value of variability calculated from the pulse rate and a combination of values.

The initial test is carried out preferably by a simplified method by means of pulse rate and the degree of arrhythmia, or also of the degree of variability, with the following ECG test in case of a negative result or directly by a complex method with ECG. In the initial test by the simplified method the sinusoidal course is evaluated, what means in case of the zero degree of arrhythmia that the result is OK, or arrhythmia occurs, if the arrhythmia degree is not zero, what means negative result i.e. the NG result. In case of arrhythmia, the arrhythmia degree is evaluated, from the neglected one to the high one. One degree of arrhythmia is considered to be 10% of the of the irregular pulses from the total number of pulses, i.e. the first degree corresponds to 10%, the fifth degree to 50%. The highest, the tenth degree corresponds to the theoretical value of 100%. In words, the degrees of heart rate, i.e. the number of heart beats per minute, is preferably expressed as follows: <35 very low pulse rate, 35-40=low pulse rate, 40-45=lowered pulse rate, 45-85=normal pulse rate, 85-100=increased pulse rate, 100-115=high pulse rate, >115=very high pulse rate.

In words, the degrees of arrhythmia are preferably expressed e.g. as follows: Ar=0 none; 0<Ar≤0.5 low; 0.5<Ar≤1 increased; 1<Ar≤2 high; 2<Ar≤3 very high; 3<Ar extremely high arrhythmia.

In words, the degrees of health condition are preferably given by variability, they express preferably the following: >7=excellent; 5.0-7.0=perfect; 4.0-5.0=very good; 3.0-4.0=good; 2.5-3.0=satisfactory; 2.0-2.5=unsatisfactory; 1.2=bad; <1.2 very bad.

A combination of the above mentioned values is expressed in words preferably as follows:

1. Ar=0.1<K<2 Possibility of arrhythmia of the type (see. 4,5)

2. Ar=0; K<1 Probability of arrhythmia of the type (see. 4,5)

3. Ar=0; K<2; T>100 Increased probability of arrhythmia of the type (see. 4,5)

4. T<200 Tachycardia

5. T>200 Flutter

Further, from the course of the heart rate are evaluated preferably individual extrasystols and their groups, omitted pulses, and heart arrest.

To consider the course as sinusoidal, 13% of irregular pulses is permitted, and the regular pulses are delimited by the limit curves of the regular pulse rate.

The absolutely regular pulse rate with 0 arrhythmias does not yet mean that the pulse is sinusoidal, it has to be tested with regard to arrhythmia of the tachycardia type, when the pulse rate is below 200 pulses/minute or with regard to the flutter, when the pulse rate is higher.

Therefore, not only the arrhythmia is tested with regard to the permitted tolerance, but also the pulse rate, which pulse rate, when unchanged, i.e. when it is a straight line in a graph within a certain optional number of pulses, there is a suspicion of arrhythmia, which arrhythmia is confirmed by the low variability below 2, measured within a longer period of time than what is the one for determination that the pulse rate is a straight line.

Therefore, in case the variability of the test result of the displayed element is lower than 2, it will be recommended to repeat the test, and when the result is confirmed, in particular when the pulse rate is a line, the ECG test is carried out.

Further the pulse rate above 85 is evaluated so that it is recommended to repeat the test at rest after some time.

When it is confirmed, the ECG test is recommended, so as if the pulse rate is too low, preferably lower than 45, or when pulses are missed.

When the pulse rate is tested by a simplified method by means of pulse rate and the degree of arrhythmia and variability, their combination is evaluated. In case of the variability, the value of variability lower than 2.5 is evaluated as NG, because it indicates weaker health condition with a risk of micro-sleep during driving of the transportation means or controlling of machines. This risk is increasing with the decreasing degree of variability. The numerical data are accompanied by verbal evaluation of the degree with regard to the results of the pulse rate, the arrhythmia or the variability. In case of the NG results because of a low variability, it is recommended to repeat the test after relaxation. When the NG result is confirmed, the ECG test is recommended.

In case of an ECG, apart from the state according to the initial test, the QRS complex, A-V block, PAC, PUC, AF, FLUTTER, TACHYCARDIA a and other pathological states are evaluated. Preliminarily, the evaluation is carried out automatically in the monitor or in the module or more accurately in the evaluation block, preferably in a PC, in a server, or in a PC of the remote participants, and preferably it is sent back to the module of the monitored person, or the evaluation is carried out manually by the professional health care personnel, preferably by a physician, and it is sent into the data module or it is reported to the monitored person or to the dispatcher, who carries out tests of a group by phone or by e-mail. In case the first test is negative, the next test, i.e. the second test is carried out preferably by means of the push-button.

In the long-time or the continual tests the automatic evaluation of the NG states is usually adjusted in the monitor screen and/or in the control and display module, and/or in the evaluation block with initialization of the alarm in case of their occurrence. To reach some savings in the volume of the data transmitted, the evaluation block transfers data as data doses or only the state that are off limits or only from time to time, according to some instruction of the monitored person or it is not transmitted and it is recorded on a memory medium in the monitor and/or in the module, preferably formed by an SD card, which card is copied to the PC. For evaluation, it can be copied from the PC by means of internet, what is cheaper than via a a mobile operator's network, or it is transmitted from the monitor or the module via a local WiFi network.

Preferably, the tests are used to do some evaluations for various activities. For example for driving of means of transport, in case the health condition is <2.5 it is alerted that there is a danger of micro-sleep, by means of reflexes in sport the possibility of overtraining is alerted, in case of weak health condition long after a training session generally danger of a stroke is alerted in case of some arrhythmia.

Preferably, evaluation of a long-time record is processed as some “HOLTER”. This automatic evaluation may be requested by a push-button “HOLTER” from a record lasting minutes to a record lasting months, wherein preferably it is possible to require a result of certain section of the record or of more sections, that may be selected from the record.

In case, the required period of time has another length than 24 hours, it is possible to request a recalculation of the results to a 24 hour record length, to obtain the results that are common for a 24-hour-HOLTER.

The result is processed in the monitor, or in the modules, and it is displayed on displaying element or it is printed. The monitor with the accessory is adapted for automatic processing of the long-term tests, of those of the HOLTER type.

Preferably, the long-time record of the heart activity of the monitored person is displayed, in particular the records for selected sets of the optional parameters for monitoring.

They include for example exceeding of the adjustable upper limit of heart rate “PULSE ABOVE”, exceeding of the limit of the minimal heart rate “PULSE BELOW”, arrhythmia above the adjustable value “AR ABOVE”, arrhythmia below the adjustable value “AR BELOW”, occurrence of tachycardia “TACHYKAR”, occurrence of flutter “FLUTTER”, time marks entered by the monitored person in times, when the person was not comfortable “NOT COMFORTABLE MARKER”, and premature atrial contractions “PAC”. Before a measurement the operator enters the limits of the values where applicable, limits for monitoring, i.e. for example the maximal and the minimal values. At first the operator selects in the field of settings the parameters to be displayed in each of the fields for displaying of the courses. There is an optional number of such fields on such display according to the number of the selected parameters and the planned length of the record to reach the optimal use of the display. Time is plotted on the x-axis, on the y-axis, occurrence of the monitored phenomenon, parameter, is marked by a short thin line segment. Because it is a long-time record, when in one display the time period is up to one month, the occurrences in time may be merged into one thick line or rectangle. This indicates that in such section the monitored phenomena occurred. An electronic magnifier of time is available for the operator, and the operator may change the scale as required to separate the merged records. By placing the magnifier to a specific place of the record and by selection of the scale, for example by transition from a month to a week, the observed place on the time axis is transformed into one field, and in other fields the records appear in the same time scale, but they are preceding the selected place and/or are after the selected place, so that all fields are logically linked together. Therefore the advantage consists in that the operator is quickly oriented whether and when the observed phenomena occurred. Subsequently, in few steps, the operator gets to the display of the exact period of time of the individual occurrence of the phenomenon. In this moment, the operator may switch to a record of the ECG and pulse rate curves of this moment and the immediate surrounding time period. Preferable is the display, when there are six ECG courses, one under the other, always following each other, preferably the earliest is at the bottom, the latest at the top. To the ECG courses corresponds a heart rate course, provided with six marked sections corresponding to the ECG curves so that the earliest part is on the right side, the latest on the left side. The corresponding places in both records are marked by cursor.

The testing equipment may be completed by some attached pieces of equipment of the operative control and display module the pieces being preferably additional units preferably placed on one or more wristbands. The monitor is mounted, preferably detachably, on a fastening element, preferably formed by press fasteners, on said wristband on a wrist, preferably it is connected by a short cable via a connect or via a wireless connection to the operative module, which module is placed, preferably detachably, on the same or another wristband, which wristband is preferably formed by a mobile phone of small dimensions, about 1 cm narrower than the wrist for comfortable and esthetic wearing.

Preferably, on the part applied to an arm or to a wrist, the monitor is provided with a contact area, which area forms preferably an ECG electrode sensing on first arm, and on the upper part of the monitor is provided another contact area for a finger, preferably forming an ECG electrode sensing on the other hand. Preferably, both electrodes are used to sense signals for processing to monitor pulse rate and/or ECG.

Alternatively, the electrode for the other hand is not placed on the monitor. It is placed on the wristband of the other hand and it is linked by a short cable via a connector with the monitor, or this electrode of the other hand is stuck on the chest, preferably in a place suitable for sensing of heart signals of the other hand, and it is connected to the connector by a wire. The number of electrodes may be increased by adding further electrodes for sensing of up to 12-lead ECG, in this case preferably without any use of the contact areas on the monitor. Alternatively, the contact areas are not placed on the monitor, they are placed on the bottom side of the wristband for a contact with the wrist, and on the upper side of the wristband for contact of fingers of the other hand, preferably above each other, so that two fingers, preferably the thumb and the index finger of the other hand push the upper contact areas and simultaneously push the bottom contact to the forearm or the wrist.

Preferably, the monitor comprises an accumulator, preferably adaptable for feeding of the operative module via a short cable, which cable preferably provides also for the data communication. Preferably, pieces of additional equipment are present, comprising additional units, such as for example additional accumulators, detectors, communication units, which are fastened, preferably detachably, on the wristband of the monitor, or on another wristbands placed next to the monitor.

The operative module on said wristband is provided with a displaying element, a controlling unit, and preferably with an optical front-end for sensing of pulse rate, of oxygen content in blood by passing light through the forearm skin. Preferably other pieces of the additional equipment are present, and they are placed externally on said wristband, preferably detachably, preferably on a spring clamp, or they are placed internally, so as for example communication unit, which preferably comprises a receiver operating at about 5.3 kHz and/or a “Bluetooth” unit, and/or an ANT unit, and further also other pieces of the additional equipment, such as for example an additional accumulator, preferably placed extractably in a case for this accumulator and/or further for example a voice unit, preferably fixed detachably on said wristband, preferably by means of a spring clamp 9.

The operative module, which is preferably of smaller size, communicates by means of wireless link with the module, which is preferably of larger dimensions and preferably is formed by a mobile phone or a tablet, and said modules are adaptable for receiving and displaying of data, in particular on phone calls, or on the SMS messages, preferably of those done by means of a SIM card present in the module, and preferably initialized in the operative module. Preferably, the operative module is adaptable to receive incoming phone calls or SMS messages and/or is adapted for realizing the calls in the “hands free” mode, or by putting the phone on an ear after the phone has been disconnected from the wristband, or by means of a voice unit adapted for putting it on an ear, preferably detachably fastenable to the operative module, or to its wristband, and communicating with the operative module via a wireless connection. It comprises an accumulator, preferably a replaceable one, a microphone, a speaker, and a control unit with an amplifier. Preferably, voice is sensed by means of a microphone built in the operative module, so that transmitting of the sound onto the voice unit is only one-directional to the speaker, and preferably for the wireless communication one-directional radio signal of a limited reach is used, which signal does not require any coded hand shaking of the communication and is undemanding with regard to energy consumption, and provides for listening. In this case, the microphone is preferably placed in the module. Alternatively, instead of the voice module, a detachable module is used for the communication, which module is put on ear after removal. Preferably, a connector is present in the mounting element for fixing and releasing of the module, which connector allows a disconnectable connection of the connectable units. The module is adaptable to operate independently without any module. In this case it comprises preferably a SIM card for operation as a phone.

Preferably, the operative module is adaptable for connecting of other external pieces of the additional equipment, such as for example of a detector of temperature, pulse rate, and oxygen content in blood, which detector is preferably fixed on a finger, or a pressure meter preferably fixed to an arm or a wrist.

Preferably, the attachment equipment comprises additional units placed preferably on individual wristbands interconnected by a short cable or via a wireless links with the operative control and displaying module.

The so sensed pulse rate, is processed preferably by the operative control and display module, or the control and display module to obtain values and/or curves of the pulse rate, arrhythmia, variability, or the operative module is preferably adaptable to process the pulse rate sensed for example by the monitor.

A compact design of the attachment equipment is advantageous, wherein the attachment units are detachably attached to each other by a mounting element, and a connector is serving simultaneously for interconnecting of the attachment equipment with the operative module by a short cable or via a wireless connection, or je attachment equipment placed on the operative module is connected directly by a connector without any short cable and by mounting elements.

In a preferable embodiment, the monitor is built in the operative module provided by contact areas on the upper and the bottom sides of the monitor for a contact with the forearm of one hand and with fingers of the other hand, wherein preferably, they are replaceable by a contact area on said wristband of the other hand or by a stuck electrode on the chest, or the contact areas are replaceable by two electrodes stuck on the chest. Alternatively, the contact areas are situated on the bottom and on top of the wristband for contact with a forearm of one hand and with fingers of the other hand. The contact areas, preferably the electrodes are connected preferably by a short cable to the front-end of the ECG and control unit of the monitor for processing, and from there, they are transmitted into the operative module, preferably for displaying. Alternatively, the monitor is connected disconnectably to the operative module by means of a connector and a mounting element.

Preferable is a set of the operative module and a monitor, which are placed on wristbands on first arm, which set is completed by a sleeve sensing the blood pressure on the same arm, and also with a finger detector of oxygen in blood, where the pressure meter and the of oxygen in blood finger detector are connected by a short cable or via a wireless connection into the operative control and display module, which module is connected preferably to the monitor for displaying data about ECG or pulse rate. In particular, if the monitor is not used, preferably with the operative module, it receives alternatively values of pulse rate from the detector instead from the monitor.

Preferably, Alternatively, the pressure monitor senses the pulse rate from the pressure in the measuring sleeve for sensing of pressure, which pressure fluctuates in the pulse rate rhythm, where decreasing of pressure in the sleeve is preferably delayed for a longer record of the pulse rate, or after reaching the low pressure value pressure in the sleeve is again increased to a level, when the pulse is measurable to reach a longer record of the pulse rate value. Data from the pressure meter are sent to the operative module placed on an arm or a wrist by means of a short cable or a wireless link.

The so sensed pulse rate is processed, preferably by the operative module or the module to values and/or curves pulse rate, arrhythmia, variability or the operative module is preferably adaptable for processing of the pulse rate sensed for example by the monitor. The monitor and/or the module and/or the pressure meter are preferably connectable with the module for displaying of data and the measurement results. The pressure meter may be controlled from the control and display module. Similarly, another meter sensing health state data from the body of the monitored person is connected.

In another embodiment the pressure meter is preferably placed on the pressure sensing sleeve on a forearm or a wrist or the pressure meter is placed on a wrist separately from the sleeve, and it is connectable by a tube, or it is placed, preferably detachably, a monitor or the monitor is built in the pressure meter. The ECG electrode of first arm is placed on the bottom side of the sleeve to contact first arm and the other contact area, preferably situated on the surface of the monitor or from above on the sleeve, preferably forms the electrode for fingers of the other hand and is used to sense heart signals, preferably the pulse rate or the ECG. The electrodes are connected to the monitor for processing of the heart signals. The control unit of the pressure meter processes data about pressure and sends them further to the operative module, into which module the data on pulse rate or the ECG are also sent from the monitor. Preferably, data may be displayed also on the displaying element of the pressure meter. In the operative module, data is processed and stored in memory and/or displayed on the displaying element and/or transmitted to the module and/or to the block for evaluation. Preferably, data from the detector of oxygen in blood, placed preferably on a finger, and from the sensor of body temperature, connected by a short cable or via a wireless connection are also transferred to the operative module. Preferably, when monitoring the arrhythmia or the extrasystols or the left out pulses, the heart rhythm is transmitted by an induction 5.3 kHz connection, which connection is energy saving, and only in case of errors of the monitored quantities with regard to an adjustable standard complete ECG data starts to be transmitted by a connection, which is to transmit it, for example a Bluetooth connection, which connection exhibits high power demand. Preferably, some Bluetooth Low Energy, or another principle is used, which is low power demanding if only pulse information is transmitted, and it demands more power if data transmitting necessary for the ECG transmission has been started. Evaluation of pathological phenomena, such as arrhythmia, from the pulse curve instead of from ECG, to monitor such phenomena, is not only advantageous with regard to the power consumption, but another advantage is also that a longer period of time may be checked. It is enough to transmit the ECG curve only for a moment of time, when there appear data that are off the adjustable limits. Preferably, the ECG curve is not transmitted, but stored continuously in memory of the monitor, to allow that in case it is necessary, the curve can be transmitted not only from the moment when an error was found in the pulse curve, but as any optional section before this event backwards. Storing of data is preferably carried out using a portable memory medium, preferably a SD card, to make possible to transfer the data not only electronically, but also by moving the SD card and inserting it into the selected equipment. So the monitor operates as a HOLTER monitor with optional length of the recording, which may be used for some k long-time monitoring, even for months.

Preferably, the testing equipment is configured as one which is placed on a stand and/or on at least one Rotoped, wherein a pressure meter, oxygen detector in blood and monitor are connected by a short cable or via a wireless connection directly or to the module, which is preferably placed on stand or it is on the Rotoped. The data is transferred from the module preferably to the evaluation block, and evaluated preferably they are transferred back to the dispatcher's PC, which is preferably localized at the stand or they are sent to the monitored person. Mounting of the contact areas and of the above specified units is preferably adaptable to other suitable configurations than on the stand.

The stand or the Rotoped with the chest belt with the independent monitor or with the there moved one from the stand is adaptable preferably for measurement and evaluation of blood pressure, oxygen content in blood, temperature, pulse, ECG the from them derived quantities, including the variability, arrhythmia, and all functions and errors in the heart activity that can be detected from them, such as for example PVC, PAC, AV block, omitted pulse, heart attack, arrhythmia, flutter, bradycardia, tachycardia.

They are evaluated automatically, preferably either in the monitor, module, where they may be even displayed on the displaying element 1, or they are processed more precisely in the evaluation block, from which block they are preferably sent back to the module of the monitored person or to the dispatcher's PC and as data for displaying on the displaying element and/or for storing in memory, or they are evaluated by a physician and communicated by voice, preferably via a phone communication or by an email or by another suitable way. Preferably, the described equipment can be placed also in some means of transport, e.g. in a vehicle or in an aircraft.

The operative control and display module is preferably placed in the mechanism for its detachable mounting, which is preferably formed by a magnetic holder of the operative control and display module, which is preferably formed by a mobile phone or a mobile watch, which are preferably provided with a SIM card for phone operation. The mechanism for detachable mounting is equipped with a system of magnets, preferably formed by a magnet, which may be preferably doubled or tripled for higher holding force, wherein the counter-magnets are placed in the module body. Preferably, the system of magnets is formed by at least two magnets, the magnet and the second magnet placed on the holder body with oppositely oriented counter-magnets, a counter-magnet, and a second counter-magnet on the module body in such configuration that when the module gets into a near but incorrect position, there is a strong force acting so that the module is automatically pulled into the correct position in the holder, and in this position the monitor is snapped into the holder in such a way that always a pair of magnets with opposite poles is tightly joined. Preferably, the join is also secured by a mechanical flap, which can be used for securing of the module position in the holder in extreme conditions, for example in doing some sport.

The flaps may swivel in hinges, are arrestable in both positions, the open one and the closed one, preferably with a notch on the hinge axis with opposite protrusion on the hinge body. Preferably, the flap is controlled by thumb and index finger. For better grip of the flap during opening is provided with a protrusion.

Similarly, the mobile wristwatch may be inserted in the smaller mechanism.

Advantageously they communicate with another mobile phone, which phone they preferably control, receive or initialize phone calls, display the phone number of the calling person or the called person, the SMS reports, a survey of the realized or not received calls that were realized or were not realized from this mobile phone and/or the watch, and data from the mobile phone operated as a module, moreover, the wristwatch preferably receives health care data from about the body of the monitored person via a mobile phone or directly and preferably they pass them to the mobile phone. When a phone call is received they communicate as a standard phone or if taken away the phone can be put near to an ear and to use the speaker of the watch functioning as headphones and microphone. Preferably, the wristwatch is operated independently, without any module, as a mobile phone detachable by means of a mechanism with a wristband and preferably they receive health data about the body of the monitored person, and preferably control the monitor and sensors. Preferably, the wristwatch is provided with headphones on its bottom side for putting them to an ear after their extraction from the wristwatch, and on the upper side is a speaker, which can be heard also when it is inserted in the mechanism. The wristwatch is adapted to be operated as a module, and for a mobile phone they are adaptable by parts of the mobile phone to operate a module, and then they are provided with all parts and features.

In an alternative universal embodiment of the monitor the monitor is put on the control and display module, preferably formed by any mobile phone with a cross-connecting connector of the mobile phone, preferably some USB connector, connected with a data connector of the monitor, whereby a mechanical and electrical connection of both parts is realized. An advantage of the universality is the possibility to connect it with most of the common mobile phones, and when the monitor is removed, it can be put on another equipment. Preferably, this connection is supported by auxiliary mounting, preferably stuck by means of protrusions, preferably for sticking to a spare module cover of the module 358, which is preferably formed by the mobile phone, from which the monitor is detachable. Preferably, the spare cover is replaceable in reversal by the removed original cover of the mobile phone, which phone was, after putting on of the auxiliary mounting, stuck on the spare cover. On the body of the monitor there are two contact areas serving as electrodes for fingers of the left and the right hands to sense the heart signals, preferably to obtain an ECG curve. Alternatively, the areas for sensing of said heart signals are adapted for applying also to the chest, and they are placed on cover of the mobile phone. Said areas forming electrodes are connected with the monitor by means of a short cable of the electrodes to the chest and by a connector of electrodes. Preferably, said areas and the short cable are preferably fastened on a sticking strip, with which they are stuck to the spare cover, wherein the overhanging short cable is inserted into the connector. When the monitor is disconnected, the connector is during a transitional period of time preferably placed in a small pocket on the sticking strip. The electrodes for chest may be used also to sense the heart signals from fingers of both hands. Preferably, the so connected monitor does not increase the thickness of the mobile phone.

The monitor of this design is powered by the mobile phone, and it communicates with it by data transfer by means of a cross-connecting connector of the mobile phone or it communicates with it by means of a wireless link, preferably formed by a Bluetooth. An advantage of the direct connection of the monitor to the control and display module is that the monitor is powered from it and does not need any own accumulator, and it communicates via a connector and does not need any wireless module, e.g. some Bluetooth, what allows smaller dimensions.

This monitor can be inserted into the base of the monitor, with which it is connected by a cross-connecting connector. The base of the monitor is provided with additional equipment, preferably by an accumulator of the base of the monitor, which is preferably replaceable aby a charged one, a communication module of the base, preferably formed by a Bluetooth, and by the parts as required, that provide the monitor for different use, and the fastening elements of the base, preferably represented by press fasteners, serve for both mechanical and electric connection to the chest belt or wristband snap plate, shortened chest belt, which can be held on the chest without any spanning by an elastic belt. It is made of a rigid material, preferably a flexible one, e.g. of rubber or of a plastic, to make possible that it is applied to the chest, where for good contact with skin it is held by hand, preferably in the place of the monitor snapped on the press fasteners.

The belt is provided with a U-shaped profile, which is used to push the chest belt by means of the pressure belt.

So, the monitor may be used for sensing of heart signals both from the contact areas of the chest belt and the electrodes, or the electrodes for fingers, to sense the heart signals in case the chest belt is not connected.

Placement of the monitor base on the wristband, placed on a wrist of the monitored person, preferably communicating wirelessly with the control and display module, preferably formed by a mobile phone and/or communicating with the operative control and display module formed preferably by a mobile phone on a wrist is preferable, wherein one electrode for sensing of the heart signals is formed by a contact on the wrist of one hand, which is placed preferably on said wristband, and the other electrode of the other hand is formed by one of the contact areas placed on the body of the monitor. Alternatively, the other sensing electrode is placed apart from the monitor, and it is connected to it by a wire via a connector of electrodes, and it is placed preferably on the wristband on the other hand as a contact on the wrist of the other hand, or preferably it is firmly fixed by sticking as a contact stuck on the chest of the monitored person in a place suitable for sensing of the heart signal of the opposite part of the body, and therefore, of the other hand. The monitor is connected to the base of the monitor fixed on the wristband with the contact of the first hand on the upper surface of the monitor, and the contact of the other hand, the contact placed on the wristband, connected via press fasteners with the bottom part of the base. The monitor communicates with the control and displaying module by means of a wireless link.

The module, preferably the detachably placed on the wristband on a wrist, is adapted to receive the incoming phone calls to the module. Alternatively, the operative module is placed on the wristband non-detachably, and it is provided with a voice unit, preferably detachably fastened on the wristband, which communicates with them or with the module and realizes phone calls by means of a microphone and headphones.

Preferably, the base of the monitor is connected by means of a connector to the contact of the other hand, preferably formed by an electrode stuck on the chest, or preferably formed by an electrode in the wristband of the other hand, and wireless communication of the monitor base with the control and display module. The base is adapted for wire connection of two or more electrodes, preferably stuck to chest to broaden the ECG sensing up to 12-lead one.

One design of connecting the electrodes to the connector of the monitor base connected with the monitor by a connector is that for the contact of the first hand are used small surfaces on the bottom side of the monitor and for contact of the other hand are used surfaces on the upper side of the monitor, or an electrode stuck on the chest. The base communicates with the module wirelessly.

Advantageously, the monitor is inserted in the monitor base next to it, on the wristband with a contact of the other hand on the bottom part of the monitor.

Another advantageous embodiment is that the monitor is placed removably in the monitor in case, wherein the case shape is adapted for perfect connection with the control and display module, preferably formed by a mobile phone, or with its cover to form a compact unit, advantageously by gluing the protrusions of the case to the cover or by forming a prolonged cover, which substitutes the original cover so that this design is optically one unit. The electrodes for sensing of the heart signals are placed on the cover of the controlling and displaying module, preferably formed by a mobile phone, preferably connected to other connector of the electrodes.

Preferably, the bridging accumulator is placed in the monitor case and makes possible to feed the control and display module, preferably formed by a mobile phone, when its main accumulator is replaced for a charged one, which replacement is thus carried out preferably without any interruption of the module operation.

To link the monitor with the control module, preferably insertion into the cross-connecting connector of the control module, preferably formed by a mobile phone on said wristband, is used. The electrode of one hand for the wrist is preferably placed on the bottom part of the monitor, the electrode for fingers of the other hand is placed on the upper part. The view in the D direction shows the holder for extractable mounting of the monitor fastened by protrusions to the cover of the control module or it is carried out as a prolonged cover, which replaces the original cover of the module.

The monitor is powered, and it communicates with the control and display module by means of the cross-connecting connector or it communicates with it by means of a wireless link, preferably formed by a Bluetooth module.

When the monitor is without any physical connection with the mobile phone, the set is placed in a plug-in-holder.

The independently placed monitor is connected by a cross-connecting data connector of the monitor to the base, preferably provided with an accumulator of the monitor base and/or with a communication module, which is preferably formed by a Bluetooth communicating with the control and display module, preferably formed by the mobile phone, and/or the operative control and display module, preferably formed by a mobile phone preferably placed on the wristband. Further, the monitor base is preferably provided with units as required, preferably with a SD card, with parts for communication via a mobile operator's network, with a SIM card, with an optical front end for sensing of the heart pulse, preferably with a fall detector, a receiver and/or a transmitter for wireless link in the 5 kHz band and with other units of the attached module. Preferably, the monitor is provided with own control unit. Preferably, various monitor bases are connected to the monitor as required with various equipment. Preferably, the fastening elements are formed by the press fasteners and allow fastening of the monitor case with the monitor to the chest belt or to the wristband.

The plug-in holder is formed to as to allow to insert in it the monitor.

Preferably, the case 1021 prolongs the base and is of the monitor form.

Connecting of the monitor with the control module is solved preferably by means of a cross-connecting bridge, where the monitor is connected via a bridge and the first connector of the bridge, where the second bridge connector is connected to the control and display module, which is formed preferably by a mobile phone. The monitor is placed in the attached cover, wherein it is put in it preferably in the disconnected state. The additional accumulator is also here, and via a short cable connected to the bridge, it feeds and/or charges the module and preferably also the monitor.

The accumulator is removably placed in the attachment cover by means of a fastening device, which device makes replacement possible, preferably without interruption of the module operation. Preferably, the fastening device comprises a door for easy extracting of the accumulator.

The monitor is extractable from the attachment cover and when extracted it can be used for connecting to the monitor case with an accumulator, what replaces the feeding from the accumulator or the module. To save place, the monitor is preferably without the accumulator and the wireless module, which are preferably present in the attachment cover. The additional cover is put on the module, preferably instead of its own cover. The attachment cover is preferably provided with contact areas for fingers or for putting them on chest that are connected by a short cable to the connector of electrodes.

Design of the monitor is also preferable in that the monitor is connected to the control module, preferably formed by a mobile phone, by plugging it into the cross-connecting connector of the module by a data connector of the monitor preferably placed as eccentric to form a space for placement of the control unit of the display. The contact surface areas are situated preferably on a side and on the bottom when viewing the display.

The monitor is provided with a display unit, which unit is formed by a control unit of the display and a small display. The display unit can be connected to the monitor by means of a connector placed on a side so that it forms a compact whole with the monitor and the module. The monitor is provided with a test start push-button.

Another embodiment, the one with the large display, is solved so that the monitor is inserted into the cross-connecting connector, and its display unit is provided with a large display, the size of which is nearly across the whole face side of the monitor. Preferably, the monitor is integral with the control unit of the monitor. The control unit is visible when the display module is observed.

In another embodiment of the monitor model, the monitor is designed for a specific module, wherein its cover is modified so as to solve connecting and shared covering both of the monitor and the display unit. The cover is provided with contact surface areas to be used for sensing of the first hand signal and the other hand signal either by touching them with fingers and/or by putting the cover to the chest of the monitored person.

Another model of the monitor, which is used for short-time tests, when the monitored person intentionally touches the contact area for a short time is such, that the monitor is put into the monitor base, which base comprises a control unit of the base, an accumulator of the monitor base, a double cross-connecting bridge, and press fasteners for connecting and fastening of the monitor according to the previous embodiment. The bridge is provided with a connector interconnecting, into which the monitor via its connector and the monitor base via the base connector are connected. The monitor is provided with contact areas for sensing of signal of one hand and the signal of the other hand.

Advantageous is also the alternative when the bridge is formed by a triple bridge, when the connector connections provides also connection of the control unit of the display and of the big display by the connector of the display.

In another similar embodiment a small display is used, which is placed at the control unit of the display, whereby lower construction height of the whole set is obtained.

In another solution the monitor is connected via a data connector of the monitor to the operative control and display module, which is placed detachably on the wristband. The monitor is provided with sensing contact surface areas arranged so that it is possible to sense the signal of one hand by the contact surface area on the bottom side of the monitor, and the signal of the other hand by touching with fingers of the other hand the other contact surface area placed on a side of the monitor body.

In another similar solution the monitor is provided additionally with a control unit of the display and with the small display, with which the connector in the cross-connecting double bridge is connected, which is element placed between the monitor and the operative module. Alternatively, the large above the control unit placed display is used.

In case a long-time test is necessary, during placement of the control and display module and the monitor on the wristband, the monitor is provided with a connector for electrodes to be used for wired connection of the electrodes sensing the signal of one and/or of the other hand by electrodes or by contact surface areas. The electrodes are placed either on wristbands on the wrists and/or they are stuck on suitable places of the body. Alternatively, the electrode of one hand is solved by means of a contact surface area on the bottom side of the wristband. In case of the wired connection of the electrodes the operative module is fixed in its position on the wristband. Therefore, for the possibility of communication with the monitored person by means of the operative module a detachable voice unit is used, which unit is placed detachably on the wristband and is connected with the operative control module wirelessly, preferably via a Bluetooth and makes possible to make a “hands-free” call.

A monitor model provided with monitor base and a transmitter that can be located is advantageous. This principle may be applied for any design of the monitor. The transmitter transmits with constant transmitting level, preferably in time intervals to save energy. Its position can be located according to the signal strength by a directional antenna connected to a measuring receiver. According to the signal intensity this measuring receiver preferably evaluates approximate distance to the located transmitter. By cooperation of two such receivers it is possible to locate the position of the transmitter as the point of intersection of both obtained directions. This transmitter for locating the position is used, when the GPS system is not available, when the control and display module transmits information about current position, preferably by means of the mobile operator's network or via WiFi.

Advantageously the monitor is placed on the monitor base, which base is fastened to the belt placed on body of the monitored person by means of a case or a spring clamp, preferably fastened to the base by means press fasteners. To sense the heart signals some above mentioned contact areas are used, preferably two stuck electrodes connected to the monitor by wires by means of a connector for electrodes. The number of electrodes may be increased to a greater number to receive up to 12-lead ECG.

Another advantageous embodiment consists in the multiple use of the monitor in connection with Rotopeds, that transmit, preferably wirelessly, by means of the provided control and display modules and monitors current results of pedaling, preferably at least “the mileage”, speed and time, preferably to a shared screen. When the person rides up to the finish which is adjustable by the number of kilometers according to the number of pedal rotations at the adjustable load, time is measured on individual Rotopeds, and thereby the order of racing persons. If handicaps are used different loads are adjusted according to performance of the racing persons. Simultaneously, health data and the heart activity are preferably displayed on the control and display module.

The monitor is provided with mounting elements that simultaneously allow receiving of heart signals, and it is transferrable to various pieces of the auxiliary equipment, such as to the chest belt, the snap-on plate with contact areas, control elements.

If the main monitor is inserted into the base it acquires all features of a self-sufficient monitor. The monitor cooperates with various cooperating units, in particular with the control and display module, the operative control and display module.

### BRIEF DESCRIPTION OF THE DRAWINGS

FIG. 1 shows a monitor of cardiac signals inserted in a box and fixed to a chest belt

FIG. 1 det. 1 shows an opened door for short-term monitoring

FIG. 1 det. 2 shows how to take a monitor out of the box for long-term monitoring

FIG. 2 shows a monitor detachably connected to a chest belt using mounting elements

FIG. 2 det. 1 shows a disconnected monitor

FIG. 2 det. 2 shows a monitor inserted in a housing and snapped to a mounting

FIG. 2 det. 3 shows a monitor with a plate conductively snap-fitted to mounting elements

FIG. 2 det. 4 shows a monitor with a snap-fitted plate, inserted to a housing

FIG. 2 det. 5 shows a monitor inserted to a housing with an opening enabling access to contact areas

FIG. 3 shows a monitor connected using a connector

FIG. 3 det. 1 shows a side view of a controlling and displaying module

FIG. 3 det. 2 shows a monitor connected to a module using a connector and slip-on housing

FIG. 4 shows a monitor placed on a control element

FIG. 4 det. 1 shows a block diagram how to connect a monitor to a controlling and displaying module

FIG. 4 det. 2 shows options how to position a monitor

FIG. 4 det. 3 shows a module of auxiliary units that can be inserted to a controlling and displaying module or to a monitor

FIG. 4 det. 4 shows options how to place a monitor on a one-track vehicle

FIG. 4 det. 5 shows gloves with contact areas inside the gloves

FIG. 4 det. 6 shows a monitor fixed to a glove or wristband

FIG. 4 det. 7 shows how to place a monitor in a controlling and displaying module

FIG. 5 shows a module of auxiliary units placed in a monitor or module

FIG. 6 shows a controlling and displaying module placed detachably on a wristband

FIG. 7 shows a display element consisting of a display with curves and numerical values displayed on the element

FIG. 8 shows a chest belt with a monitor under clothes and a pressure belt

FIG. 8 det. 1 shows a pressure belt with a single winch in a backrest

FIG. 8 det. 2 shows a pressure belt with its flexible part, wearable also outside the vehicle

FIG. 8 det. 3 shows a chest belt with electrodes and a monitor under clothes

FIG. 8 det. 4 shows a chest belt with a monitor advantageously fixed using snap fasteners.

FIG. 8 det. 5 shows a pressure belt whose correct position is ensured by U-shaped profiles

FIG. 8 det. 6 shows a pressure belt split in half whose halves can be linked using a buckle and tongue

FIG. 8 det. 7 shows a chest belt adjusted to be fixed to a pressure belt using a bridge

FIG. 8 det. 8 shows a pressure belt with a chest belt fixed on a single spot using a bridge

FIG. 8 det. 9 shows how to insert a chest belt through unbuttoned clothes

FIG. 8 det. 10 shows how to move a chest belt already inserted under clothes in the direction of the B arrow to the centre of the chest, with subsequent buttoning the clothes up

FIG. 8 det. 11 shows a chest and pressure belt with magnets used to fix their connection

FIG. 8 det. 12 shows implementation of seat belts led diagonally across the chest, using two pressure belts

FIG. 8 det. 13 shows a combination of a seat belt led diagonally across the chest with a pressure belt

FIG. 8 det. 14 shows a seat belt led diagonally across a chest and used simultaneously to to press a chest belt inserted under clothes

FIG. 9 shows a testing device

FIG. 9 det. 1 shows a display element of a monitor consisting of a display

FIG. 9 det. 2 shows waiting display when a button has been pressed

FIG. 9 det. 3 shows testing display with pulse, ECG, arrhythmia and variability values and/or curves

FIG. 9 det. 4 shows resulting display of a test summarizing an OK good state

FIG. 9 det. 5 shows a “NO GOOD” bad state requiring an action

FIG. 9 det. 6 shows how to display a result of a temporary test pressing a button during long-term testing

FIG. 9 det. 7 shows how to start a test automatically by putting hands on electrodes during short-term testing

FIG. 9 det. 8 shows detailed initial display

FIG. 9 det. 9 shows a detailed example of waiting display after a button has been pressed

FIG. 9 det. 10 shows how to display the course of a test after having initialized the test

FIG. 9 det. 11 shows how to display resulting display with test results after the test has finished

FIG. 9 det. 12 shows a “NO GOOD” negative result

FIG. 9 det. 13 shows how to display a negative result and test results evaluating the pulse, arrhythmia and physical condition

FIG. 9 det. 14 shows an example how to display a long-term record of monitored effect occurrences of cardiac signals of the monitored person

FIG. 9 det. 15 shows how to display ECG records for the moment searched within long-term records of monitored effect occurrences

FIG. 10 shows an auxiliary device of a controlling and displaying operative module consisting of auxiliary units fixed on one or multiple wristbands

FIG. 10 det. 1 shows an auxiliary device consisting of auxiliary units fixed on separate wristbands

FIG. 10 det. 2 shows a compact version of an auxiliary device where auxiliary units are mounted detachably to each other using a mounting element and connector

FIG. 10 det. 3 shows an auxiliary device connected directly to an operative module using a connector without a cable and mounting elements

FIG. 10 det. 4 shows a monitor embedded to an operative module contact areas on both upper and lower side of the monitor

FIG. 11 shows a pressure meter on a wristband and an oxygen in blood detector which are connected to a monitor placed separately and an operative module placed on a wristband, using a cable or a wireless connection

FIG. 11 det. 1 shows a pressure meter placed on a pressure sensing sleeve, forearm or wrist with a monitor and operative module fixed on it, alternatively connected to the sleeve using a tube

FIG. 12 shows how to connect a pressure meter, oxygen in blood detector and monitor using a cable or a wireless connection directly or via an associating unit to a module mounted on a stand

FIG. 13 shows a magnetic holder of an operative and displaying module

FIG. 13 det. 1 shows how to complete a magnetic holder of an operative and displaying module with fixing valves

FIG. 13 det. 2 shows a magnetic holder with lockable valves

FIG. 13 det. 3 shows a mobile watch with a loudspeaker, headphone and microphone, detachable from a stand

FIG. 14 shows an alternative design of a universal monitor mounted to a controlling module

FIG. 14 det. 1 shows how to connect contact areas to a monitor using a cable

FIG. 14 det. 2 shows how to store a cable after a monitor has been disconnected

FIG. 14 det. 3 shows how to connect contact areas to a monitor using a detachable cable

FIG. 14 det. 4 shows how to connect contact areas to a monitor using a strip with contact strip strings

15 shows a monitor that can be inserted to a monitor base

FIG. 15 det. 1 shows a shortened chest belt

FIG. 16 and its det. 1 shows how to place a monitor base on a wristband

FIG. 16 det. 2 shows details how to connect a monitor base

FIG. 16 det. 3 show a monitor in a base, placed on a wristband

FIG. 17 shows a design when a monitor is detachably placed in a shape-adapted monitor housing

FIG. 18 shows a monitor plugged to a cross-connecting connector of a mobile phone controlling module on a wristband

FIG. 19 shows a monitor connected to a base using a cross-connecting connector

FIG. 20 shows a monitor connected using a bridge

FIG. 21 shows a design when a monitor is connected to a controlling module by plugging to a cross-connecting connector

FIG. 21 det. 1 shows a monitor featuring a displaying unit

FIG. 21 det. 2 shows a monitor design with a displaying unit with a big display

FIG. 21 det. 3 shows monitor characteristics designed for a specific module

FIG. 22 shows an optional variant of a monitor for short-term testing when the monitored person intentionally touches the electrodes for a short time period

FIG. 22, det. 1 shows an extended version with a triple bridge

FIG. 22, det. 2 shows a similar version with a small display

FIG. 23 shows a monitor solution connected to a controlling and displaying module placed detachably on a wristband using a cross-connecting connector

FIG. 23 det. 1 shows a monitor design with a display control unit

FIG. 23 det. 2 shows a monitor design with a big display

FIG. 23 det. 3 shows a monitor that can also perform long-term tests

FIG. 24 shows a variant of a monitor with a locatable transmitter

FIG. 25 shows a monitor placed on a belt

FIG. 26 shows three rotopeds and a shared screen

FIG. 27 shows a basic monitor design for connecting with a controlling module using a connector

FIG. 27 det. 1 shows how to connect a basic monitor to a monitor base

FIG. 27 det. 2 shows a self-supporting monitor which can be moved across auxiliary devices

FIG. 27 det. 3 shows a self-supporting monitor with a fixed connection to an auxiliary device

FIG. 27 det. 4 shows a monitor built in a controlling and displaying module

FIG. 28 shows how to use a movable monitor for 12-lead ECG

FIG. 28 det. 1 shows an electrode system

FIG. 28 det. 2 shows electrode configurations for 12-lead ECG

FIG. 28 det. 3 shows a detailed solution for electrode distribution on a chest belt

### BEST MODE OF CARRYING OUT THE INVENTION

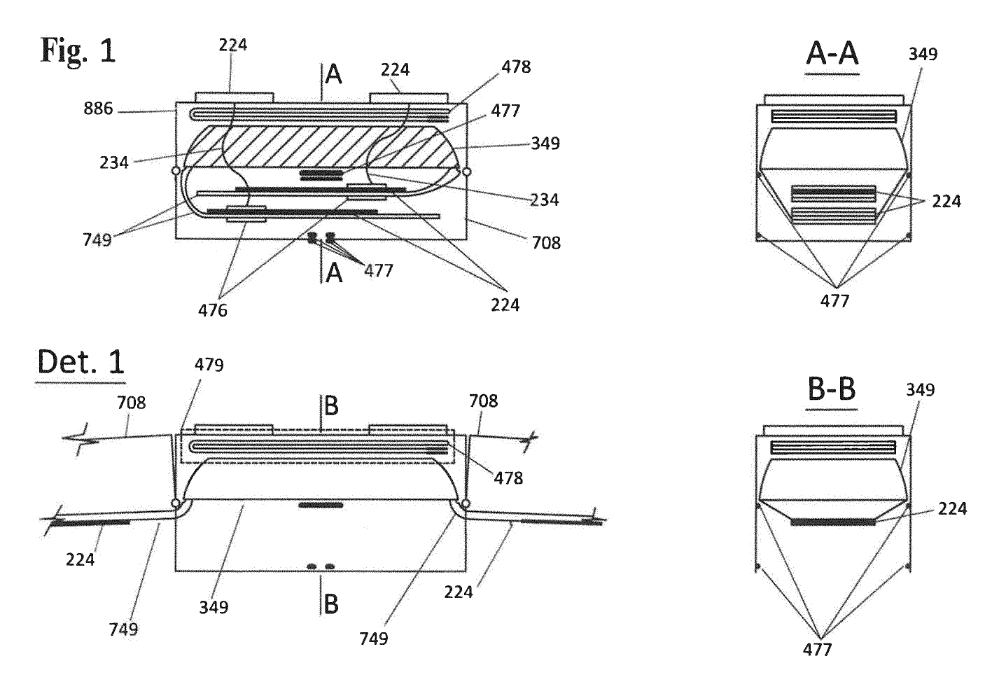

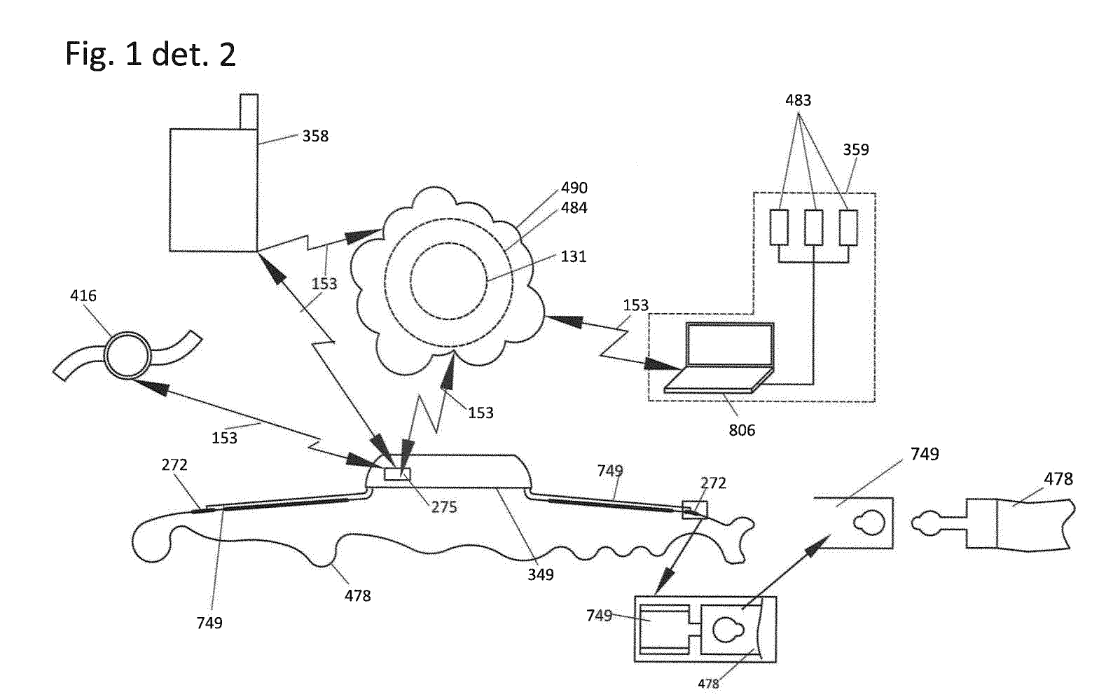

FIG. 1 shows a monitor 349 of cardiac signals inserted in a housing 886 and attached to a chest belt 749 with contact areas 224 on a belt with skin advantageously used as cardiac signal electrodes conductively connected to the monitor 349. This monitor 349 is protected against falling out of the housing 886 using backstops 477.

A chest belt 749, advantageously made of a rubber material, is inserted by folding both sides to a housing 886 and secured using a hinged door 708 against unfolding. This door 708 is advantageously secured against opening using backstops 477.

Detachable clips 476 on electrodes conductively placed on contact areas 224 are connected, using short cables 234, to ECG electrodes 143 placed outside a housing 886 to put on fingers of both hands or to be put on patient's chest for short-term monitoring.

FIG. 1 det. 1 shows an opened hinged door 708 for short-term monitoring when both sides of a chest belt 749 are unfolded, for grasping by both hands or to be put on a chest to achieve a better contact than putting fingers on contact areas 224.

FIG. 1 det. 2 shows how a monitor 349 with a chest belt 749 is removed from a housing 886 for long-term monitoring purposes. Advantageously, an elastic strap 478 is attached to this chest belt, advantageously removed from a storage space 479 shown on det. 1. Using it, a monitor 349 is attached to a chest to detect cardiac signals using contact areas 224 on a housing.

A monitor 349 processes cardiac signals and sends data as cardiac pulses or an ECG curve using a communication unit 275 via a wireless link 153, advantageously formed by bluetooth, to be displayed in detail and processed immediately or from memory to a local controlling and displaying module 358, advantageously formed by a mobile phone 100. Or, it sends the data directly or via a controlling and displaying module 358 to a remote evaluation block 359, advantageously in the form of a server 806, advantageously using a data network 490 advantageously in the form of a WiFi 131 or mobile operator 484 network. A module 357, 358 or a monitor 349 switch between a WiFi 131 and mobile operator network 484 automatically, or a monitored person does this task manually based on coverage; the WiFi 131 network has a priority to cut costs and preserve an uninterrupted connection to the server. When cardiac signals are monitored, a monitor 349 advantageously uses a power-saving communication module 22 and advantageously low energy bluetooth and sends cardiac signal pulses, preferably uninterrupted, and sends cardiac signals tier an ECG curve only when required by the monitored person or the server or a selected participant or periodically or when set limits of health-care data are exceeded. Selected participants 483 have access to a server 806, advantageously using an internet link 360.

This communication unit 275 connects advantageously a monitor with a watch 416 for operative displaying.

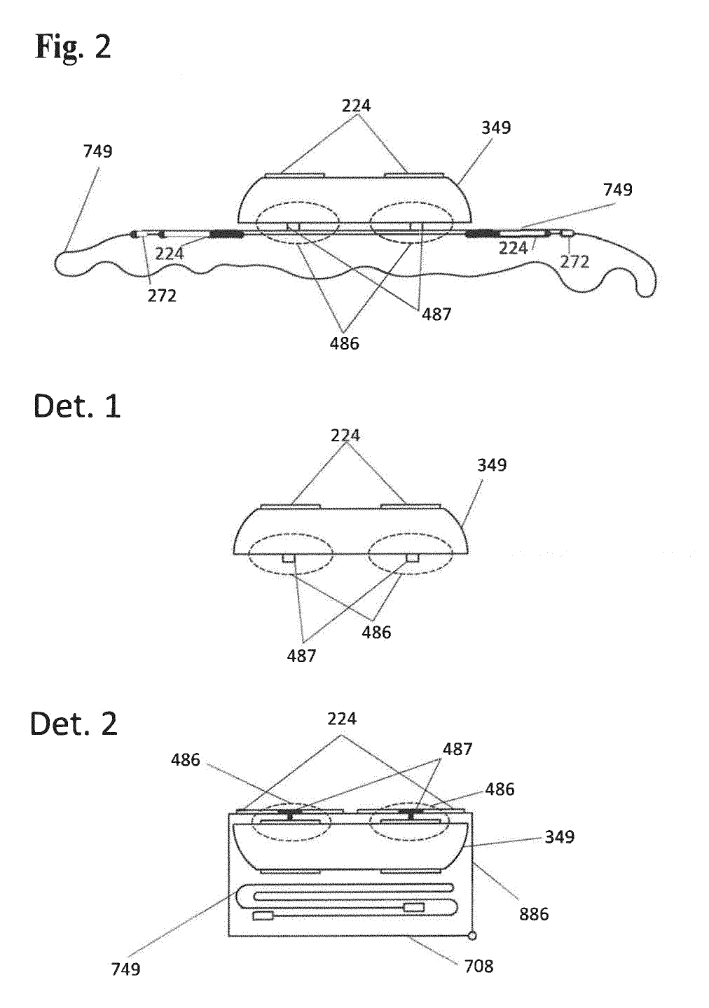

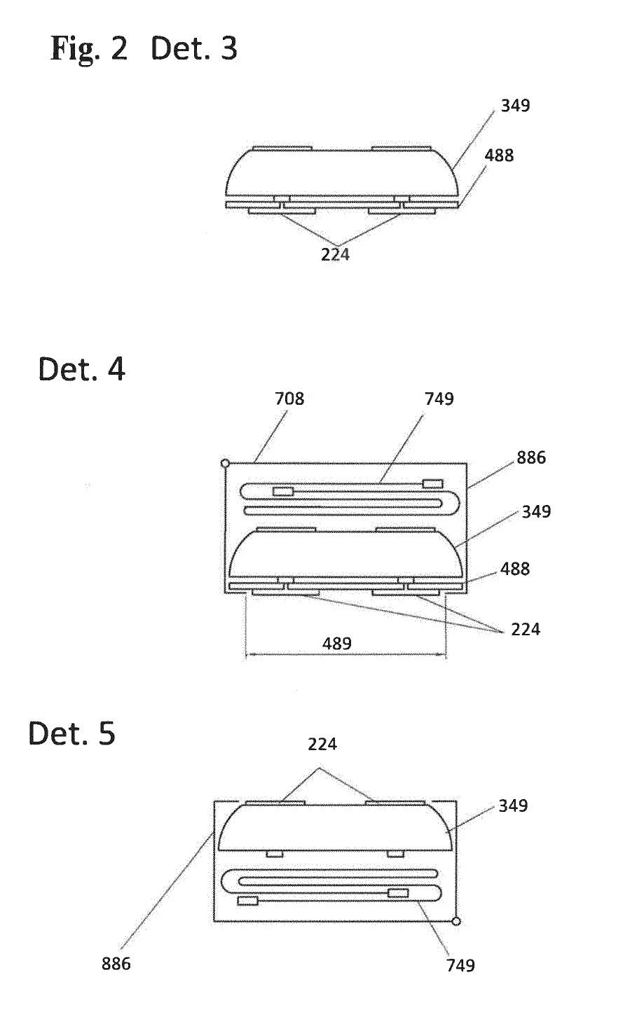

FIG. 2 shows a monitor 349 connected detachably to a chest belt 749 using mounting elements 486, advantageously in the form of snap fasteners 487 for long-term scanning cardiac signals from a chest.

FIG. 2 det. 1 shows a disconnected monitor 349. Contact areas 224 are advantageously situated on this monitor 349, to be used to scan cardiac signals by putting on fingers or being put on a chest without connecting to a chest belt 749.

FIG. 2 det. 2 shows a monitor 349 inserted to a housing 886 and snap-fitted to mounting elements 486 advantageously in the form of snap fasteners 487, connected to contact areas 224 situated on the housing 886 for short-term monitoring cardiac signals by putting on a finger or being put on a chest. A detached belt 749 is stored under a door 708.

FIG. 2 det. 3 shows a monitor 349 with a plate 488 snap-fitted on mounting elements 486 connected to contact areas 224 for short-term monitoring cardiac signals.

FIG. 2 det. 4 shows a monitor 349 with a snap-fitted plate 488, inserted to a housing 886 which has an opening 489 used to make contact areas 224 accessible for short-term measuring by putting on fingers of both hands.

FIG. 2 det. 5 shows a monitor 349 inserted to a housing 886 with an opening 489 used to make contact areas 224 situated on this monitor 349 accessible for short-term measuring by putting on fingers of both hands.

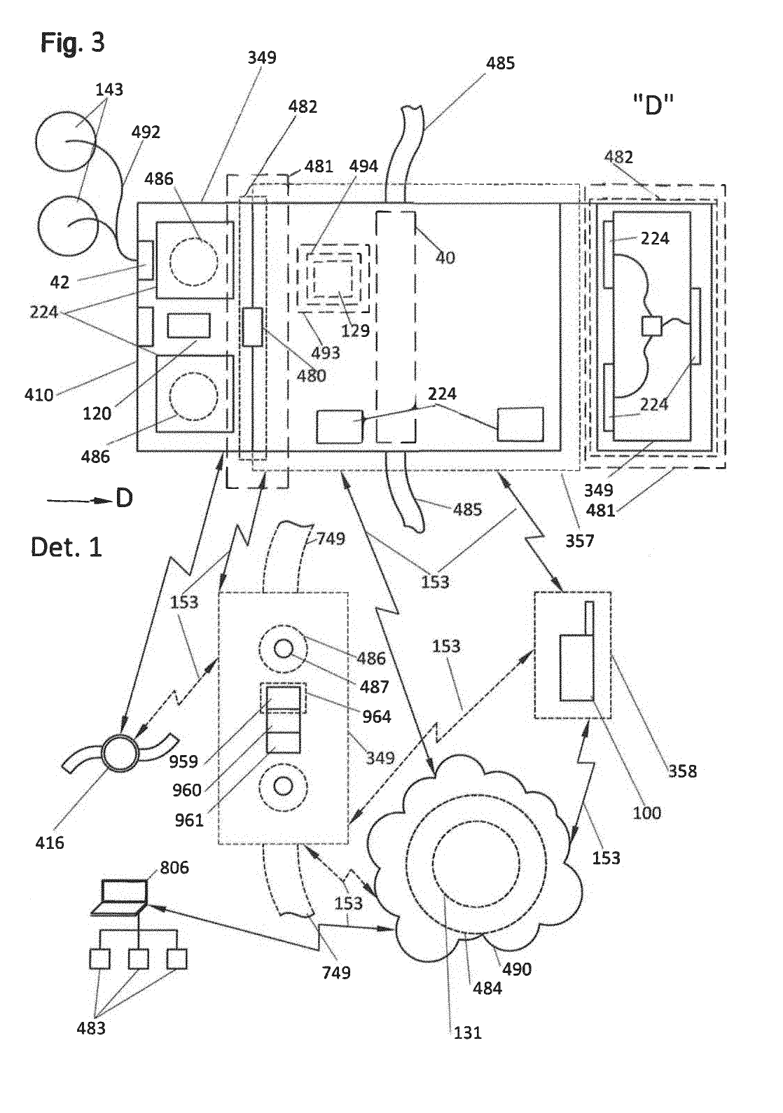

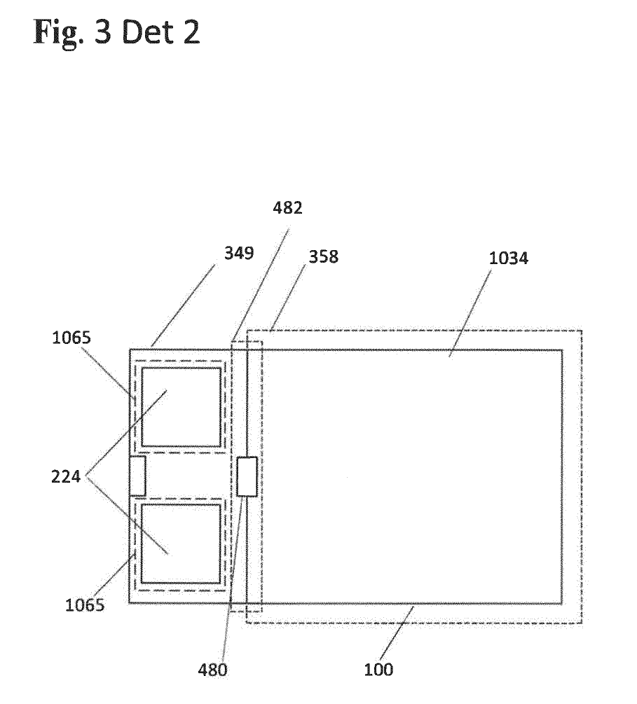

FIG. 3 shows a monitor 349, connected advantageously using a connector 480, advantageously in the form a USB connector, advantageously for data communication or power supply, from a side to a controlling and displaying operative module 357, advantageously in the form a mobile phone 100, for operative evaluation and displaying cardiac signals scanned during short-term measuring using contact areas 224 situated on this monitor 349. A monitor 349 is advantageously attached to this controlling and displaying operative module 357, using this connector 480, and advantageously for stronger attachment using a mounting mechanism 481, advantageously in the form of a slip-on housing 482 fitting closely to this monitor 349 and module 357. A module 357 is advantageously equipped with a wristband 485 to be put on a wrist, advantageously of being detachable from this wristband using a mounting mechanism 40.

Cardiac signals are scanned from contact areas 224 or by electrodes 143 whose number is optional and, therefore, scanning a single-lead ECG from contact areas 224 can be optionally extended to the required number of leads, max 12. A wrist is advantageously in contact with a contact area 224 situated on the lower side of a monitor 349, as shown in the view in the D direction. The other area 224 is situated advantageously on the upper side of a monitor 349 to be used for contact by an other-hand's finger. Both contact areas 224, optionally situated on the upper side of a monitor 349 and shown in a dashed line, are connected to a front-end 362 of an ECG monitor 349 to scan and digitalize cardiac signals. Or, cardiac signals are scanned by electrodes 143 glued advantageously on a chest and connected by a wire link 492 to a connector 42 for electrodes on a monitor 349. If a monitor 349 is disconnected from an operative module 357, it communicates advantageously with it using a wireless link 153. A monitor 342 includes a control unit 960 to process cardiac signals, a memory 961, advantageously an extractable memory medium 964, advantageously an SD card, to save scanned values of the monitored person's body as data. It enables to monitor the monitored person using currently transferred data and also, subsequently, data stored in the memory. These data are advantageously processed for a required time range, advantageously for 24 hours, advantageously to a “Holter” evaluation. You can perform monitoring advantageously for a different time range than 24 hours; the results are then recalculated to 24 hours

An operative module 357, advantageously with smaller dimensions and advantageously attached detachably on a wristband 485 for operative displaying on a wrist, communicates advantageously using a wireless link 153 with a controlling and displaying module 358, placed freely and having bigger dimensions, used for detailed display. When connected to an operative module 357, a monitor 349 uses its power and needs no additional accumulator 120. If it has an additional accumulator, it can be used as an emergency power supply during exchange of a main accumulator 129 in an operative module 357 without interrupting its operation, using a fastening mechanism 493 advantageously with a door 494.

FIG. 3 det. 1 shows a monitor 349 moved from a controlling and displaying module 357 to a chest belt 749 on a chest of the monitored person, shown in a dashed line, for long-term measuring. It communicates advantageously using a wireless link 153 with an operative module 357 and/or controlling and displaying module 358 and advantageously simultaneously with a sports watch 416. A wireless link 153 is advantageously formed by a bluetooth or ANT or inductive link advantageously approx 5 kHz. And/or, this monitor 349 communicates advantageously with a server 806 directly or advantageously via a controlling and displaying module 358 with a server 806, advantageously via a data network 490, advantageously formed by a WiFi network 131 or a mobile operator network 484 for evaluating and displaying numeric data and/or curves advantageously with a pulse with limit curves of regular pulses, arrhythmia, variability and ECG, which can be evaluated and displayed even on modules 357 and 353.

These data are displayed separately or simultaneously in optional groups.

When a monitor 349 is used separately, i.e. it is not connected to modules 357, 358 using a connector 480 and communicates wirelessly, Bluetooth low energy (BLE) technology is advantageously used and, to monitor cardiac signals, only pulse values have to be transferred, which consumes less energy than transferring a full signal for a single-lead ECG. The single-lead ECG is monitored randomly or when deviations are indicated, e.g. by a warning signal or automatic ECG display.

Limits are advantageously set, for example pulse and arrhythmia values, for which a warning signal is issued. On monitored person's wish or automatically when limits are exceeded, the device is switched to a temporary ECG transfer mode to enable determining the cause of the warning signal. This modification saves energy of the accumulator and enables longer operation without recharging than during uninterrupted transfer of ECG which is, however, recorded continuously on a memory medium 106, advantageously in the formed by a SD card. The card can be used to play the saved data and only the data exceeding the set limit values are displayed automatically. This method also saves energy and reduces necessary time to review the record.

FIG. 3 det. 2 shows a monitor 349 connected to a module 358 instead of an operative module 357 using a connector 480 and advantageously a strengthening slip-on housing 482. It has the same functions as a monitor in the FIG. 3. Cardiac signals are sensed using sensing elements 1065, advantageously in the form of contact areas 224 on the upper part of a monitor 349 for fingers of both hands. A monitor 349 is inserted into a slip-on housing mounted on a module 357 or 358 cover, glued with benefit.

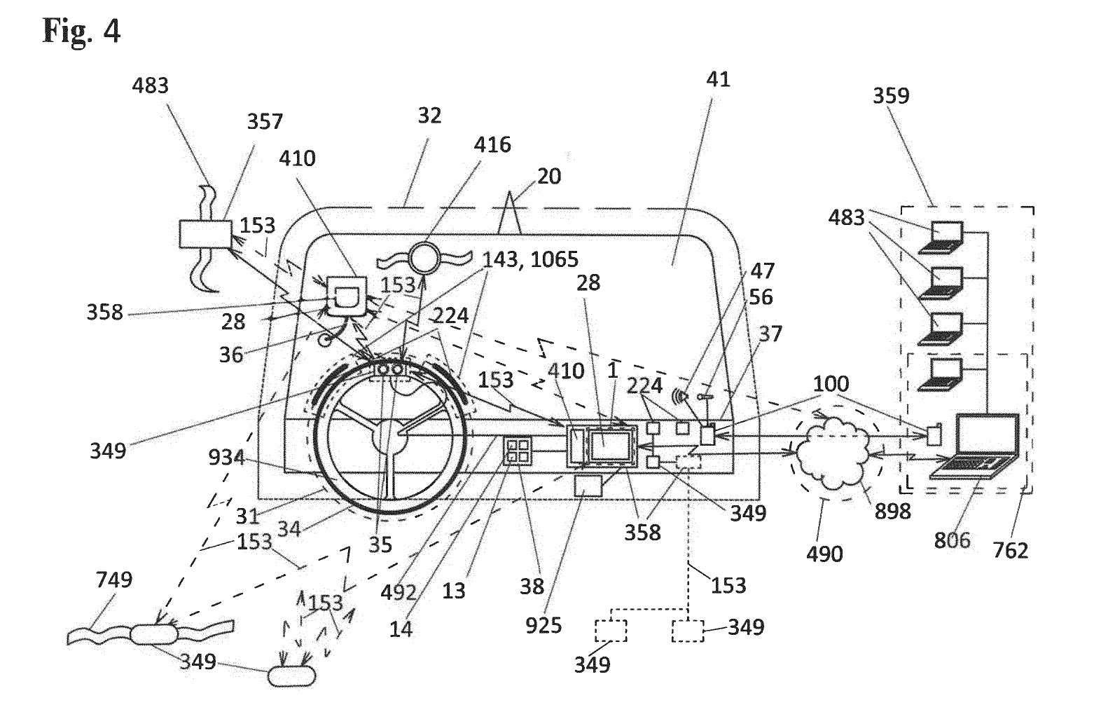

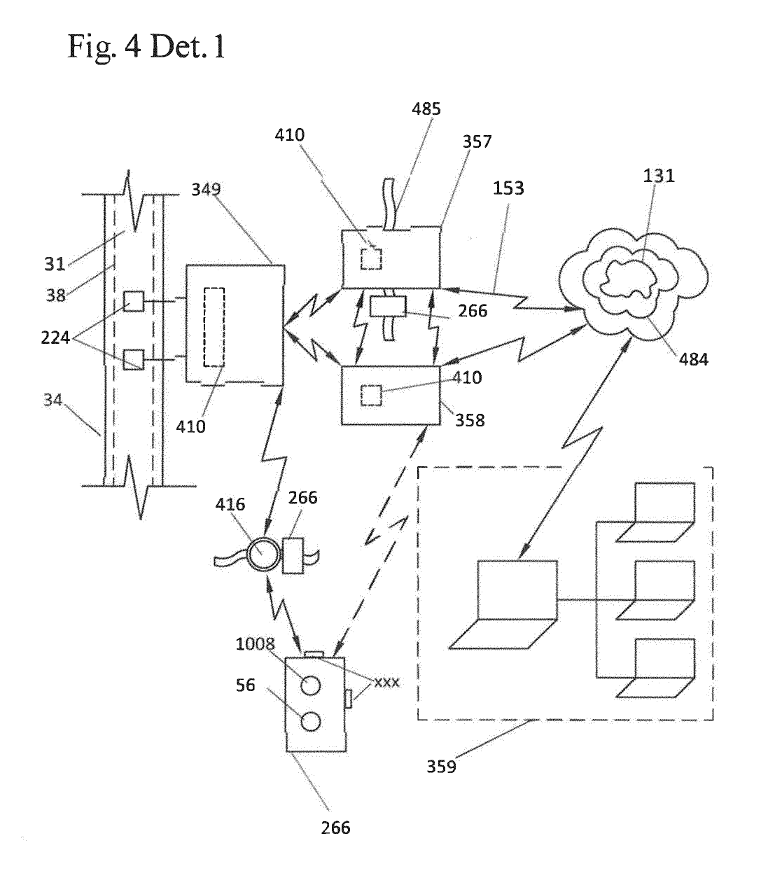

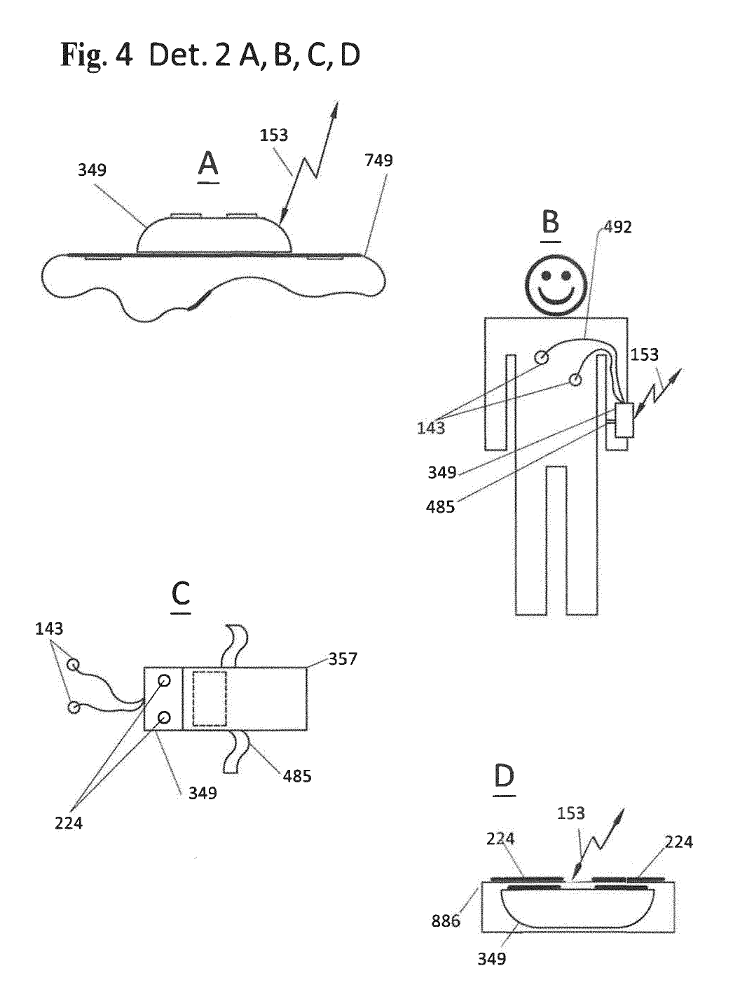

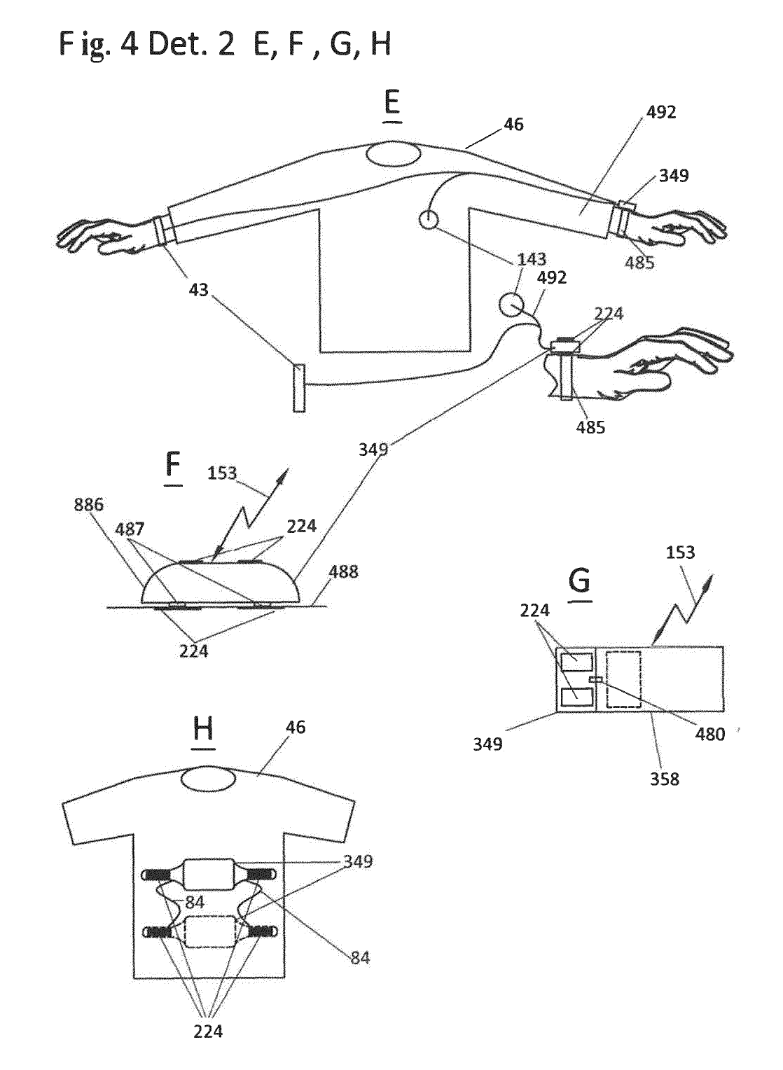

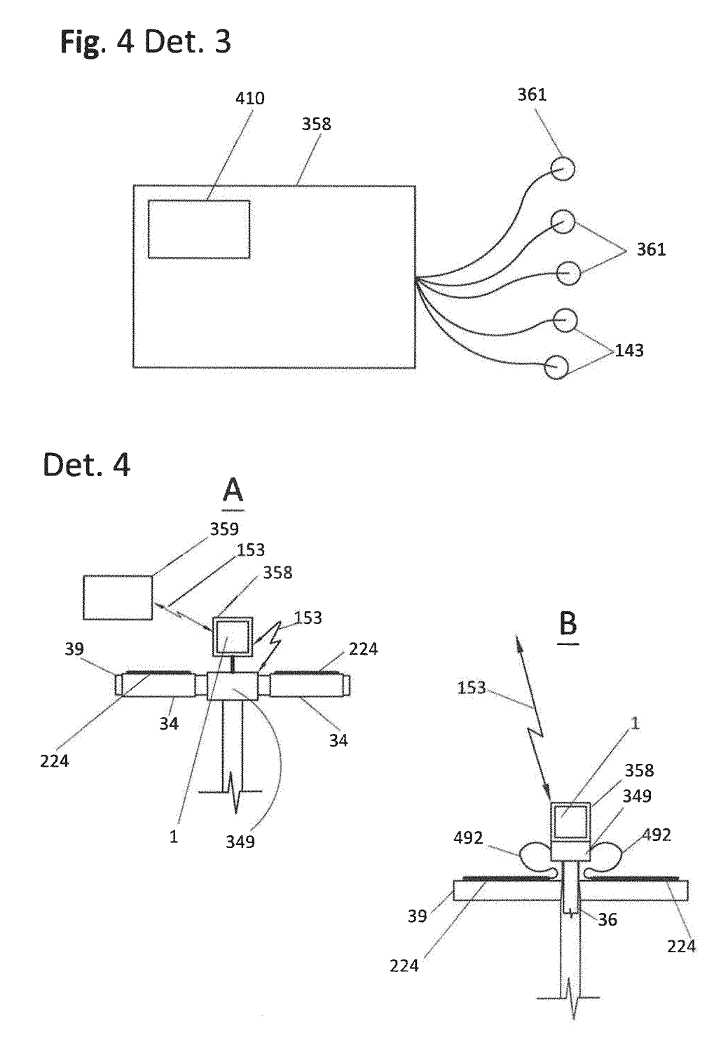

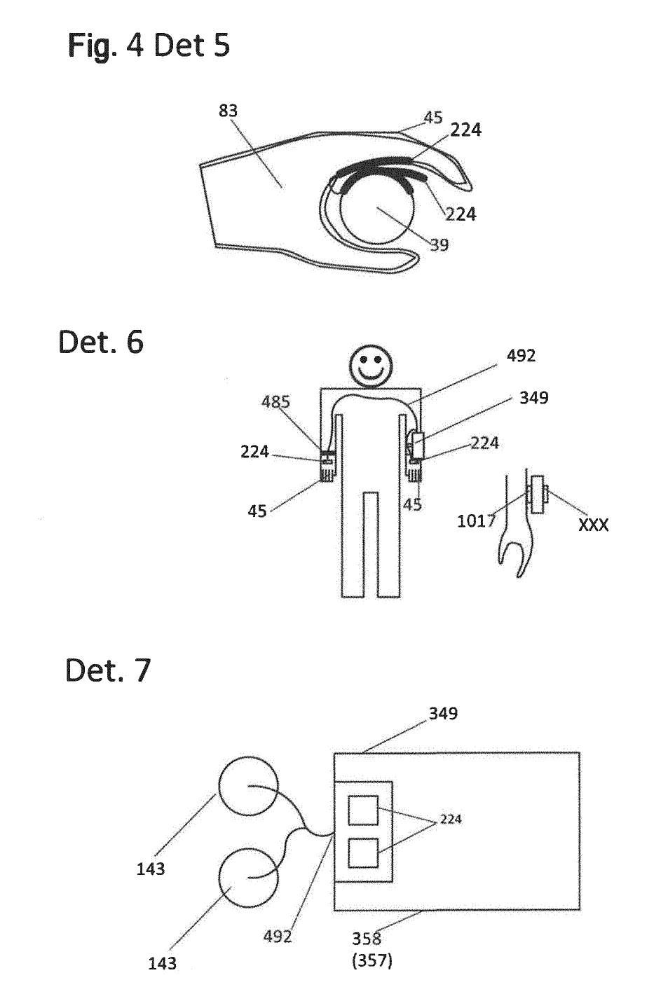

FIG. 4 shows a monitor 349 positioned on a control element 31 advantageously formed by a steering wheel 934 or steering handlebar 48, or levers 49, or a controlling element 55 of plane or push-buttons 57 of a machine or contact areas 224 of a machine of means 32 controlled by the monitored person advantageously formed by a means of transport advantageously formed by a vehicle 33 or a plane or a train or a motorbike or a bike or a full-track vehicle or a tractor or advantageously formed by a machine controlled by the monitored person.

The monitor 349 is mounted to a control element 31 in a fixed way or removably using a fastening element 35, advantageously formed by snap fasteners 487 advantageously using a sleeve 34 on which this monitor is fastened in a fixed way or removably, advantageously using snap fasteners 487.

A monitor 349 is connected to a controlling and displaying module 358 advantageously via a wireless link 153, mounted advantageously removably in a holder 36 attached advantageously removably on a means 32 preferably on a windscreen 41 or a panel 37 of the means of transport, vehicle 33 and/or connected to the module 358 built-in the means 32 preferably to the panel 37 with a controlling element 38 and with a display element 1 advantageously formed by a display 28. A monitor 349 is alternatively connected to a built-in module 358 using a wire link 492. A monitor 349 is simultaneously connected, advantageously using a wireless link 153, to a watch 416 and/or a module 357 on a wristband advantageously with smaller dimensions for operative displaying. Using a wireless link 153, a module 358 is advantageously also connected to an evaluation block 359 advantageously via a data network 490 preferably WiFi and/or via a mobile operator's network 898. A built-in module 358 is advantageously connected using a wireless link 153 to a controlling and displaying module 358 in a holder 36. These modules are, together with an operative module 357, adapted for identical displaying, advantageously based on data from a module 358 advantageously in a holder 36 or positioned freely, e. g. in a pocket.

On a control element 31, advantageously formed by a steering wheel 934 or a sleeve 34, there are sensing elements 1065, advantageously contact areas 224 advantageously formed by electrodes 143 for monitoring cardiac signals from both hands during controlling a means 32 by the monitored person for processing to a pulse value or curve and/or arrhythmia and/or variability and/or ECG. They are connected to a monitor 349 directly or via a fastening element 35 advantageously formed by snap fasteners 487. Monitoring results are advantageously processed automatically or by health-care staff on a PC of a server 806 or remote participants, and the result is sent back to the monitored person as data via a mobile data network 490 advantageously a network of a mobile operator 484 or by phone. Other passengers are advantageously monitored by another monitors 349 connected to a module 358 and, in turn, an evaluation block 359. Advantageously, an emergency call push-button 14 and a reset push-button 13, which the monitored person can use to cancel the call and it can be used in both health-care and transport emergency, are situated on a controlling element 38. An accident detector 925, monitoring advantageously an impact, roll-over, activated air bag and other events indicating an accident, is advantageously connected to a module 358. An accident detector 925 advantageously sends accident data via a module 358 to a server 806 and information about health of the monitored person is advantageously sent simultaneously, sensed using a monitor 349 or a multi-purpose chest belt 749 advantageously with a monitor 349 of cardiac signals on a chest belt or separate sensors detecting physiological values of human body like its temperature, breath, blood oxygen and other values described in other figures. A monitor 349 in a chest belt 749 is connected to a module 358 in a holder 36 or a built-in one. Alternatively, cardiac signals are sensed using a monitor 349, movable from a chest belt to a plate 488, wristband 43 of the second hand of a housing 886, or works separately with contact areas 224 situated on it. Advantageously, a state of health outside limits or an accident cause a warning signal, advantageously reset using a reset push-button 13 which is sent to the server 806 of a surveillance center, preferably, when it is not reset. When a monitored person is continuously connected to a monitor 349 or sensing elements, he/she is permanently monitored when connected to a surveillance center in an evaluation block 359, which, if the health data get out of limits, advantageously connects to the monitored person using mobile phones 100 or a hands-free data connection with a microphone 56 and a speaker 47. These are advantageously grouped with a module 358. The system sends GPS coordinates automatically during an accident or when the health data get out of limits or on request. A monitor 349 is advantageously connected to a vehicle computer for co-ordinated sharing and transmitting data to a server 806 and to an operation centre monitoring vehicle accidents. A module 358 is advantageously connected to an external GPS antenna 20 on the vehicle, advantageously situated on the vehicle roof. Advantageously, contact areas 224 are positioned on the passenger's side and connected to a monitor 349 to test the passenger.

On a controlling and displaying module 358, there is an emergency call push-button 14, which is used to send an emergency call to a surveillance center 762 and which is also used to make a call when the set the health parameters are out of limits. An emergency call is preceded advantageously by a warning signal during which the emergency call can be cancelled. When an emergency call is received, the surveillance center 762 is adapted to make a voice call to the driver and can access the health data detected by a monitor 349 or detectors in a module of auxiliary devices and, based on data evaluation, it can turn on warning lights in the vehicle and advantageously stop it or drive remotely to park the vehicle in a safe location. A means of transport, advantageously a vehicle is equipped with cameras making remote driving easier.

Alternatively, or when the vehicle is not connected to a surveillance center 762 with a possibility to drive the vehicle remotely, the vehicle is advantageously adapted to automatically switch on warning lights, stop the vehicle or drive it to a safe place to park based on GPS and Google Maps, if the driver does not reset the warning signal.

The alarm is advantageously raised even if the time set on the counter is reached if the driver does not reset the alarm in time.

A watch 416 or a module 357 on a wristband connected wirelessly to a module 358 and/or monitor 349 are advantageously adapted to be removed from the wristband for a phone operation enabled by putting it on an ear. They are equipped advantageously with components for communication via a mobile operator network or adapted for displaying and executing.

FIG. 4 det. 1 shows a block diagram of connecting a monitor 349 situated advantageously on a sleeve 34 communicating with a controlling and displaying operative module 357 advantageously in the form of a smaller mobile phone advantageously situated on a wristband for operative monitoring and communicating with a module 358 advantageously bigger for detailed monitoring and evaluation in a block 359. An operative module 357 advantageously communicates with a module 358 using a wireless link 153 and is adapted to display data from the module 358 advantageously about phone calls, SMS, and is adapted to receive calls. A module 358 is advantageously adapted for coincident displaying on module 357 display and vice versa. A monitor 349 can be advantageously installed to a means of transport in a fixed manner and connect contacts on a controlling element. A module 358 is advantageously replaced with a on-board computer which can be advantageously used to control the control means of transport, for example to park it in case of emergency call or when state-of-health parameters are out of limits.

FIG. 4 det. 2A shows how, for continuous monitoring even during the time when a means 32 is not controlled by both hands, a detachably mounted monitor 349 is advantageously removed from a control element 31 or a sleeve 34 and is mounted on a chest belt 749 as shown in FIG. 3 or in detail 2B on a wristband 485 which can be connected using a connector 42 to electrodes via a wire link 492 with electrodes 143 advantageously glued to a chest or in detail 2C attached to an operative module 357 advantageously with smaller dimensions for fastening to a wristband 485 advantageously in the form of mobile phone 100 or watch 416 for continuous scanning cardiac signals using electrodes 143 advantageously glued on a chest or for occasional scanning using contact areas 224 as already described in FIG. 3.

FIG. 4 det. 2D shows how a monitor 349 can be advantageously moved to a housing 886 for occasional monitoring.

FIG. 4 det. 2E shows a monitor 349 with one contact area 224 on its lower part on a wrist of one hand and another contact area 224 on its upper part for contact with fingers of the other hand or, alternatively, connected to an electrode 143 advantageously glued on a chest as an alternative for contact of the other hand or connected to an electrode of the other hand advantageously with a wristband 43 using a wire link 492.

FIG. 4 det. 2F shows a monitor 349 with contact areas 224 on a housing 886 or a plate 488 via a connector.

FIG. 4 det. 2G shows a module 358 with a monitor 349 contact areas 224 connected to a module 358 via a connector 480.

FIG. 4 det. 2H shows detachable positioning of a monitor 349 on clothing 46 advantageously in the form of a tee-shirt, connected using a wire link 84 to contact areas 224 situated in the chest area of this clothing. A monitor 349 shown in a dashed line is alternatively situated under a waist and connected to contact areas 224 situated in a chest area by a wire.

FIG. 4 det. 3 shows a module 410 of additional units which can be inserted to a module 357, 358 or monitor 349 to which sensors 361 are connected and electrodes 143 enable to scan and process biometric data of human body which are subsequently processed and displayed in a module 358 or an evaluation block 359.

FIG. 4 det. 4A shows a monitor 349 attached to a means 32 advantageously in the form of a single track means of transport, a bike or a motorbike, on a steering handlebar 39 in a fixed way or detachably advantageously using snap fasteners 487 or on a sleeve 34 in a fixed way or detachably. This monitor can be connected using a wireless link 153 to a module 358 mounted on a steering handlebar in a fixed way or detachably using a holder 36. Using a link 153, this module can be also connected to a watch 416 and/or evaluation block 359.

FIG. 4 det. 4B shows how to place a monitor 349 to a holder 36 advantageously with a steering handlebar 39 with contact areas 224 connected using a wire link 492 to this monitor fixed on or in a module 358 with a display element 1 and connected using a wireless link 153. A steering handlebar 39 on a holder is advantageously used to scan cardiac signals by putting on both hands.

FIG. 4 det. 5 shows an arm 83 with a glove 45 with contact areas 224 inside the gloves which ensure contact with the hand and are connected to external contact areas 224 of the gloves using a wire link 492 which ensures a conductive connection of the hands to external contact areas 224 put on contact areas 224 of a steering handlebar 39 or sleeve 34 of a single track vehicle when the gloves are put on it. Contact areas on a steering handlebar are connected using a cable 234 on a monitor 349. Or, a monitor 349 is positioned on a wrist or one of the gloves 45, on a wristband connected to a contact area in the other glove using a wire link 492. These are connected to a monitor 349 using a wire link 492.

FIG. 4 det. 6 shows a monitor 349 fixed on a glove 45 or wristband 485 and connected to a contact area 224 for contact with one hand and connected using a wire link 492 over shoulders to a glove 45 or wristband 485 with a contact area 224 for contact with the other hand to scan cardiac signals.

FIG. 4 det. 7 shows how a monitor 349 is placed in a module 357 or 358 advantageously using a mobile phone where cardiac signals are advantageously scanned via contact areas 224 situated on it or by electrodes 143 connected by wire using a wire link 492.

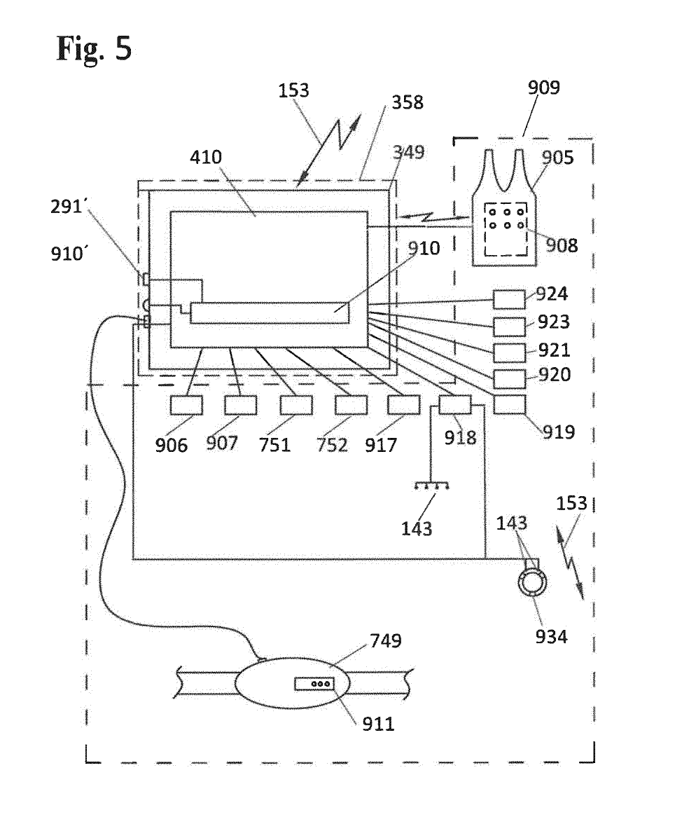

FIG. 5 shows a module 410 of additional units placed in a monitor 349 or in a controlling and displaying module 358 which advantageously contains a module 910 of internal sensors equipped advantageously with body temperature sensors 751, a pressure detector 917, a sleep phase detector 921 and a blood oxygenation detector 923. To a module 410, a set of external sensors 909 is connected, advantageously completing internal sensors which are advantageously an alcohol level detector 906, addictive substance detector 907, body temperature sensor 751, breath sensor 752, pressure detector 917, ECG detector 918, EEG detector 919, EMG detector 920 (electromyograph), sleep phase detector 921, light detector 923 advantageously detecting blood oxygenation and pulse, advantageously completing or replacing a module 910 of internal sensors which is connected to sensors 910′ placed on mobile phone 100 connected using a wireless link 153 or by wire via a connector 291′ for connecting of detectors and probes. Sensors 908 are advantageously placed on a tee-shirt 905 for sensing signals and values from selected places in the upper part of the trunk of the monitored person; they are connected by wire or wirelessly to a module 410 and complete or replace sensors in a set of internal or external sensors and/or are placed on a chest belt 749 connected by wire of wirelessly to a module 410. Sensors or external ECG electrodes 143 are advantageously placed on a steering wheel of a means of transport or other activity with a human operator, advantageously on a vehicle steering wheel, and signals from them are connected to a mobile phone using a wire link 492 or a wireless link 153. Also, ECG signals connected to an ECG detector 918 are advantageously sensed this way. A module 410 of additional units is advantageously placed in a means of transport or other activity where the driver or operator has an attached chest belt 749 with a block 911 of sensors or sensors communicating remotely using a radio link in a built-in module 410, advantageously in a module 358, and through it and a data network 490 of a mobile operator, a connection to a server 806 and a driver/operator surveillance center is implemented. Controlling and displaying for the operator/driver of the means of transport or other activity is implemented advantageously using a built-in module 358 with a touch display. Alternatively, the above described components are not built-in but they are separate.

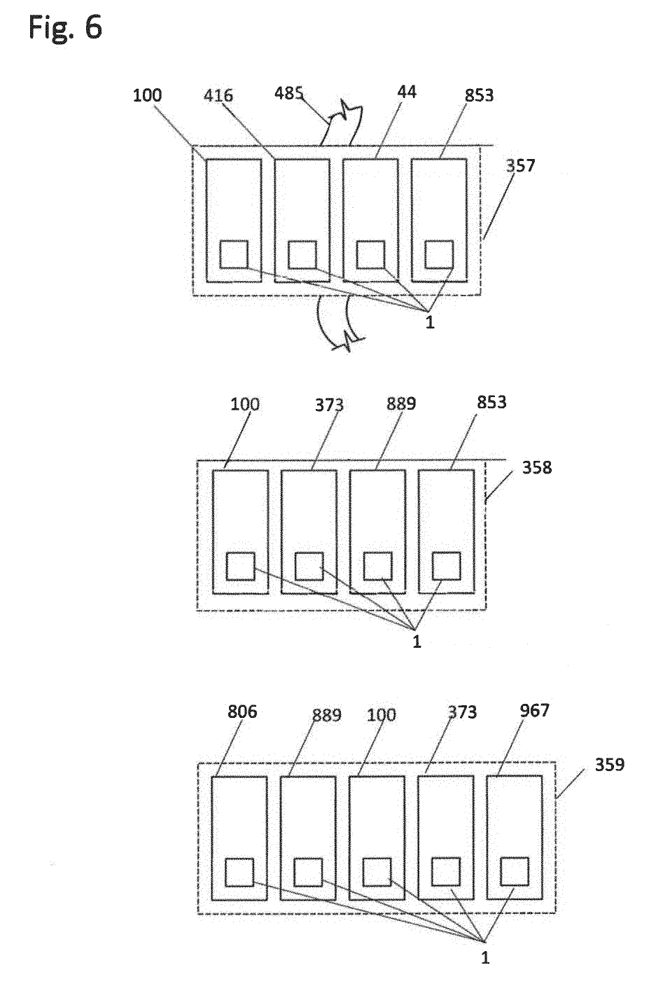

FIG. 6 shows an operative module 357 advantageously with smaller dimensions, advantageously placed detachably on a wristband 485 for operative display, advantageously in the form of a mobile phone 100 or watch 416 or monitoring watch 44 or controlling and displaying operative unit 853.

Moreover, it shows a module 358 advantageously with larger dimensions for detailed display, advantageously in the form of a mobile phone 100 or tablet 373 or PC 889 or controlling and displaying unit 853.

Moreover, it shows an evaluation block 359 advantageously in the form of a server 806 and/or PC 889 and/or a mobile phone 100 and/or a tablet 173 and/or other unit.

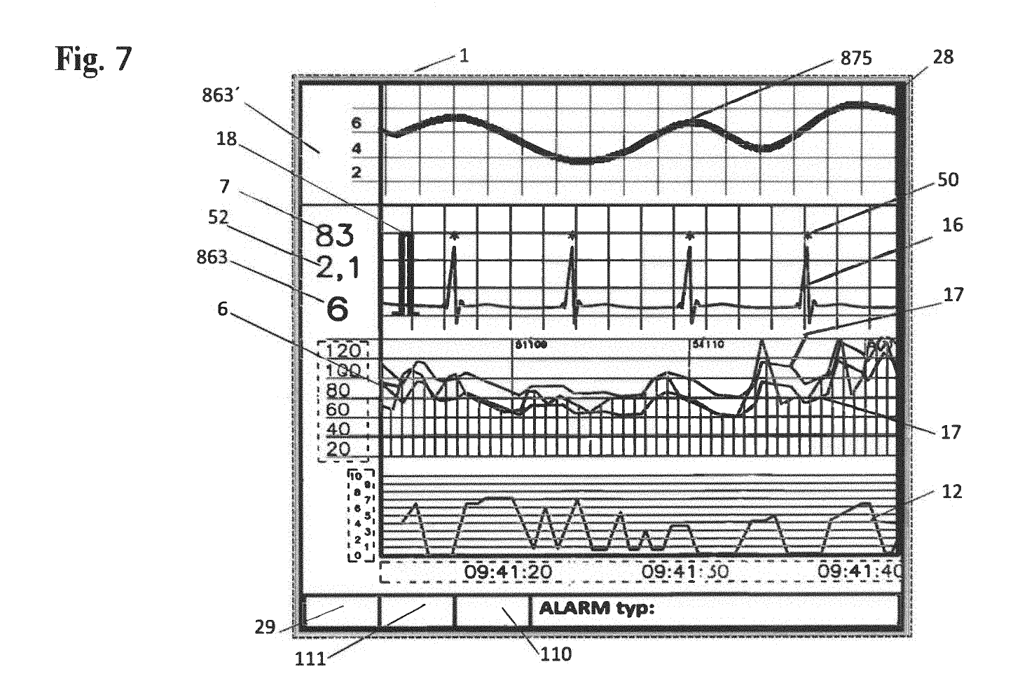

FIG. 7 shows a display element 1 advantageously formed by a display 28 with curves and numerical values displayed on it. These are, most of all, a pulse value 7, arrhythmia levels 52 in levels or % of arrhythmic pulses of the entire amount of pulses for the selected time unit, a variability value 863 in levels and pulse curves 6 advantageously with limits of regular pulses, a frequency curve 12 of the arrhythmical and regular pulses, a variability and ECG curve 875 displayed simultaneously or subsequently, individually or in optional groups.

If it exceeds adjustable limits, a warning signal is advantageously issued; if the signal is not completed with a reset button, it changes advantageously to an alarm or immediately raises an alarm sent to an optional place of destination by phone and/or as data and/or as SMS, for example to a server 806.

An alarm type that raised it is displayed together with the warning signal. If cardiac signals are sensed in the form of heart rate, then variability, arrhythmia, pulse, pulse drop-outs are advantageously evaluated.

An alarm is raised based on a too low variability value which indicates bad psychical/physical condition of the monitored person and requires caution or to avoid driving or operating means 32 or, for sportsmen, performing a sports exercise. A value like “Physical condition”, which is—for a laymen more meaningful expression indicated by the value than “Variability”, is advantageously displayed on a display element 1, advantageously on a display 28, especially for monitoring the person. A low variability value, lower than approx. 2, also indicates tachycardia or flutter, especially when the pulse simultaneously raises above a normal value measured at rest. It requires to verify regular occurrence of a single P-wave using ECG to confirm this diagnosis.

An alarm is also raised based on too low or too high pulse value above the adjustable value or when an adjustable value of pulse drop-out time is exceeded. For arrhythmia, when arrhythmia raises above an adjustable value which indicates atrial fibrillation which has to be confirmed by occurrences of a P-wave in ECG.

On contrary, when decline of a regular arrhythmia level and simultaneous sudden drop of variability are observed, flutter or tachycardia is indicated, underlined by potential pulse rise at rest, and a P-wave has to be verified using ECG to confirm the diagnosis.

An alarm can also be raised by a higher number of extrasystoles compared to an adjustable value and it is necessary to use ECG to verify whether they are/are not dangerous chamber extrasystoles which are dangerous primarily during physical activities.

Simultaneous monitoring of variability and arrhythmia is important to evaluate the heart state. A variability curve and an arrhythmia curve provide an overview of physical condition of the monitored person with regard to his/her activity in the course of time. These values are used to determine whether the monitored person is capable to drive a means 32 or to perform a sports activity and whether he/she should be examined to avoid worsening his/her state of health or to eliminate a threat to his/her life.

Pulse sensing and evaluating is performed automatically in a control unit 365 of a monitor 349 and/or a module 357, 358 and/or an evaluation block 359 and/or visually using sequential or simultaneous display of the specified values and/or curves on a display element 1. This is advantageously extended with evaluation and display of an ECG curve where atrial and chamber extrasystoles, heart attack, transfer and missing or multiplied P-wave and other pathological phenomena are evaluated.

For these diagnoses, current or subsequent display of the above mentioned values and curves is advantageously applied on a display element 1 where automatic evaluation from a control unit 365 is advantageously verified.

By evaluating the state, monitored person's capability to drive or control a means 32, advantageously of transport, is verified, and risk of accident caused by dangerous driver conditions like microsleep, heart attack, stroke, heart failure, pulse drop-out is reduced, since occurrence of these factors is, with a specific probability, indicated by a low level of variability, arrhythmias and ECG curve.

For sportsmen, monitoring these values and curves lowers health-related risks during their activities and conditions for effective training are ensured.

For monitored persons, preventive monitoring lowers risks arising from the above mentioned pathological states and supports their prevention or treatment.

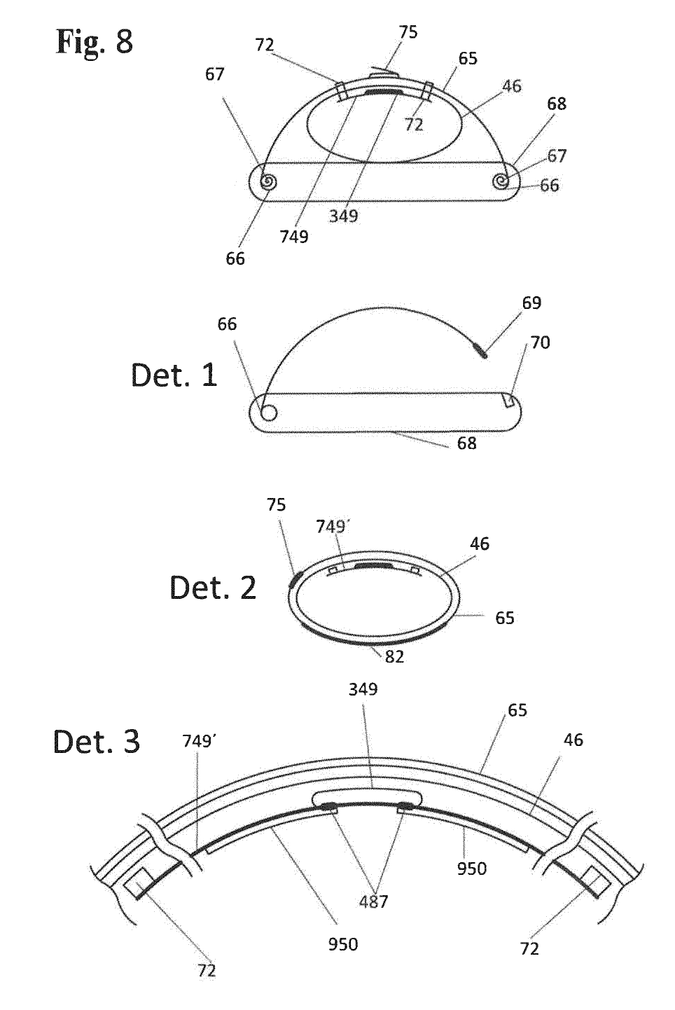

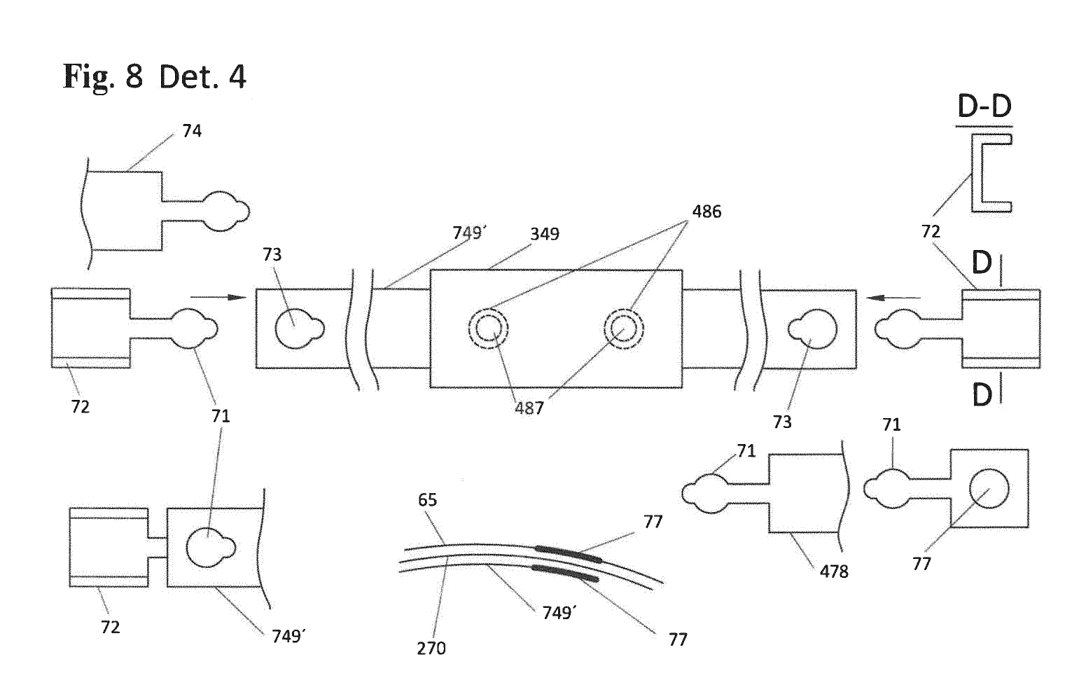

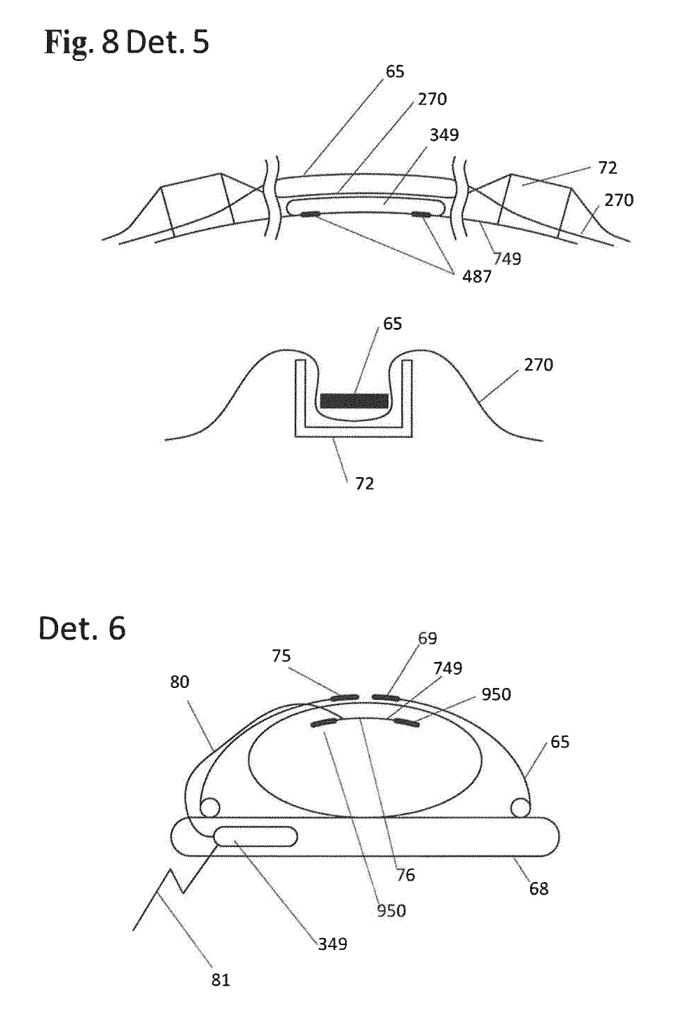

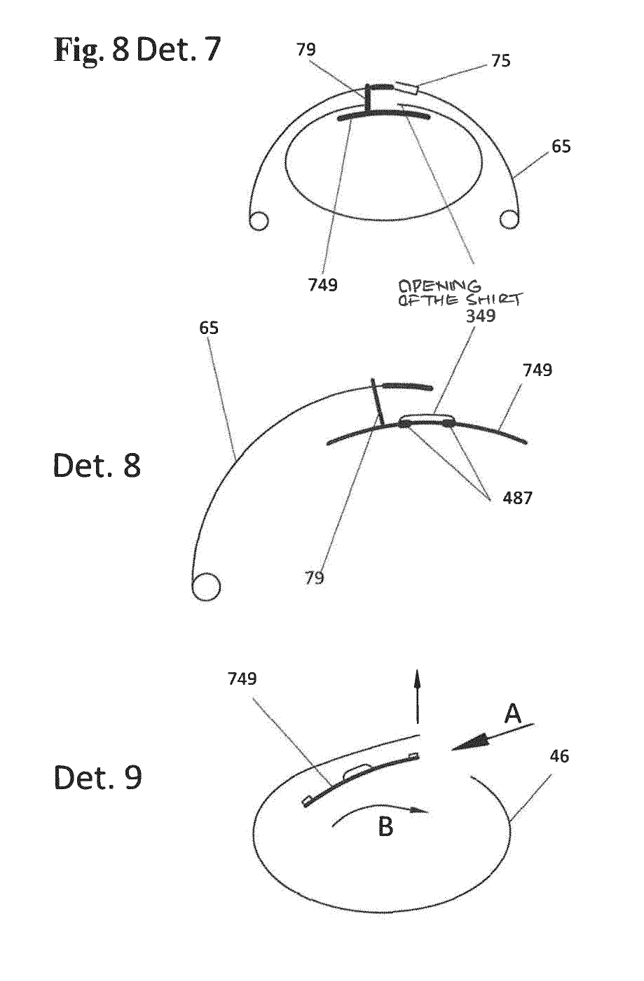

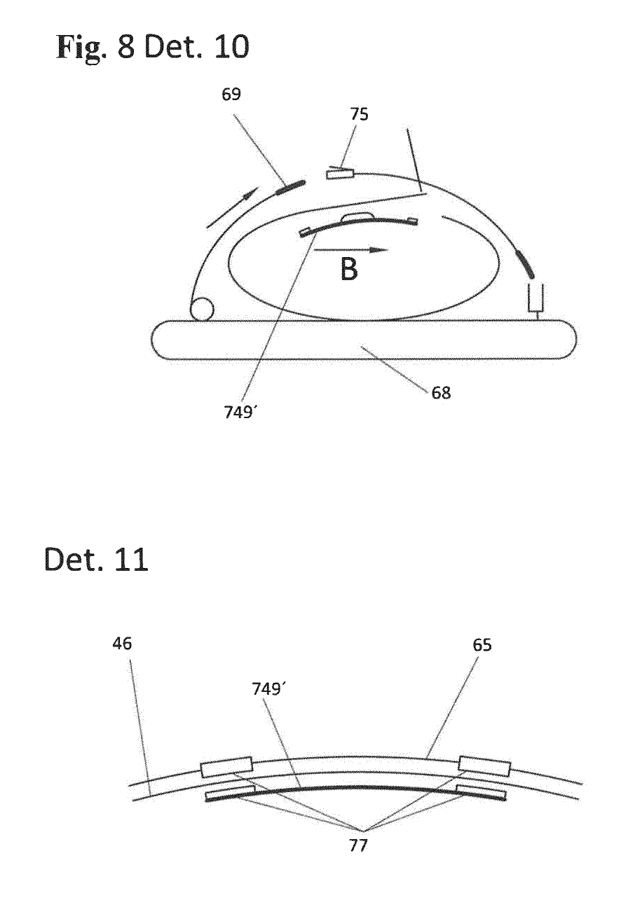

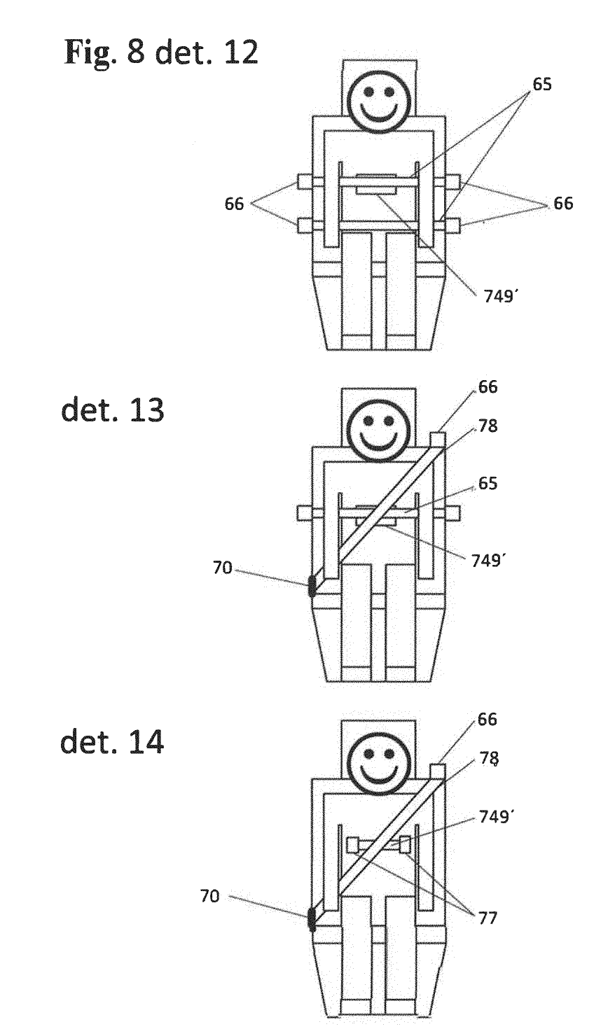

FIG. 8 shows a chest belt 749′ with a monitor 349 under clothing 46 advantageously in the form of a shirt 270 or a tee-shirt 905 pressed on the chest by a pressure belt 65 advantageously consisting of two pieces that can be combined in the centre using a buckle 75 and are winched on winches 66 with springs 67 which, after having been pulled out, winch components of the pressure belt 65 back on winches 66. Therefore, a pressure belt 65 advantageously consists of two components and each of the components is winched on a single winch installed on both sides of a backrest 68 of a seat in a vehicle 33; the components are fastened together using a buckle 75 advantageously in the centre between the winches. This design is adapted to enable a driver to move freely. When he/she moves forward or sidewards, the respective pressure belt 65 is pulled out from a winch 66 or is winched back. Springs 67 ensure the pressure belt tension to press a chest belt 749′ on the chest to scan cardiac signals. Or, the belt is pulled out of one winch and inserted on the other side to an opening of a mounting 70 using its tongue 69 with an anti-knock-out safety lock that can be released, as shown in det. 1.

FIG. 8 det. 2 shows a pressure belt 65 which fits closely to a chest and can be also worn outside the vehicle. A chest belt 749′ is pressed to the chest of the monitored person by its flexible part 82.

FIG. 8 det. 3 shows a chest belt 749′ inserted under clothing 46 advantageously in the form of a shirt 270 with electrodes 950 and a monitor 349 advantageously mounted detachably using snap fasteners 487 with extensions 71 slid onto with U-shaped profiles 72 which keep the pressure belt 65 pressing the chest belt to the chest in the position above it—the pressure belt 65 fits to the U-shaped profiles 72 through clothing 46.

FIG. 8 det. 4 shows a chest belt 749′ with a monitor 349 advantageously mounted using snap fasteners 487, with openings 73 for aligned extensions 71 with U-shaped profiles 72 or magnets which are advantageously mounted instead of an elastic strap 478 used to fix it on a chest, by fitting closely when a pressure belt 65 is used.

A pressure belt 65 with a magnet 77 is kept in the position above a chest belt 749′ by magnetic force of the magnets 77 on the chest belt situated under clothing 46.

FIG. 8 det. 5 shows a pressure belt 65 which slipped to an extension 71 with U-shaped profiles.

FIG. 8 det. 6 shows a halved pressure belt 65 pulled out of winches 66 advantageously situated in a backrest 68 whose halves can be locked using a buckle 75 and tongue 69 which can be inserted to the buckle 75 with a unlockable safety lock against sliding out. On a pressure belt 65, a chest belt 749′ is advantageously mounted, advantageously fastened using a belt 749′ advantageously in the form of a bridge 79. Alternatively, a link 80 goes through a pressure belt 65 to a monitor 349 advantageously situated in a backrest 68 from where it sends the signal via a wireless link 153 or a wire link 492.

FIG. 8 det. 7 shows a chest belt 749′ designed to be attached to a pressure belt 65 using a bridge 79 which can be inserted from outside after spreading out clothing 46, advantageously in the form of a shirt, which ensures it presses the chest belt 749′ to the chest of the monitored person. A pressure belt 65 can be locked using a buckle 75.

FIG. 8 det. 8 shows, without clothing 46 for illustration purposes, one side of a pressure belt 65 with a chest belt 749′ attached at a single spot using a bridge 79.

FIG. 8 det. 9 shows how a chest belt 749′ is inserted through unbuttoned clothing 46, advantageously in the form of a shirt, first in the direction of the A arrow.

FIG. 8 det. 10 shows movement of a chest belt 749′ already positioned under clothing in the direction of the B arrow, with subsequent buttoning clothing 46; the chest belt is now ready to be secured in the correct position by a pressure belt 65 which exerts force towards a backrest 68 and is fixed on the opposite side of the backrest 68 by a tongue 69 inserted to a buckle 75.

FIG. 8 det. 11 shows a chest belt 749 and a pressure belt 65 equipped (for fixing the contact of the chest belt 749 and a pressure belt 65) with magnets 77 instead of U-shaped profiles which are attached on both the chest belt 749 and pressure belt 65 so they affect each other through the clothing 46 and keep the chest belt in the required position on the chest.

FIG. 8 det. 12 shows how safety belts are implemented (instead of usual design of a sash leading diagonally across a chest) using two pressure belts 65 on waist and chest for enhanced security, with simultaneous pressuring a chest belt 749 to the chest by one of the pressure belts 65, preferably the upper one which serves also as a safety belt whereas the second belt 65, preferably the lower one, is advantageously only a safety belt.

FIG. 8 det. 13 shows a combination of a safety belt 78 leading diagonally across a chest with a pressure belt 65 leading horizontally across the chest to press the chest belt which is advantageously used as a safety belt for enhanced safety.

FIG. 8 det. 14 shows a safety belt 78 leading diagonally across a chest which is also used to press a chest belt 749 inserted under clothing 46 and locked in the position advantageously using magnets 77 on a chest belt with counterparts advantageously positioned on the outer side of the clothing 46.

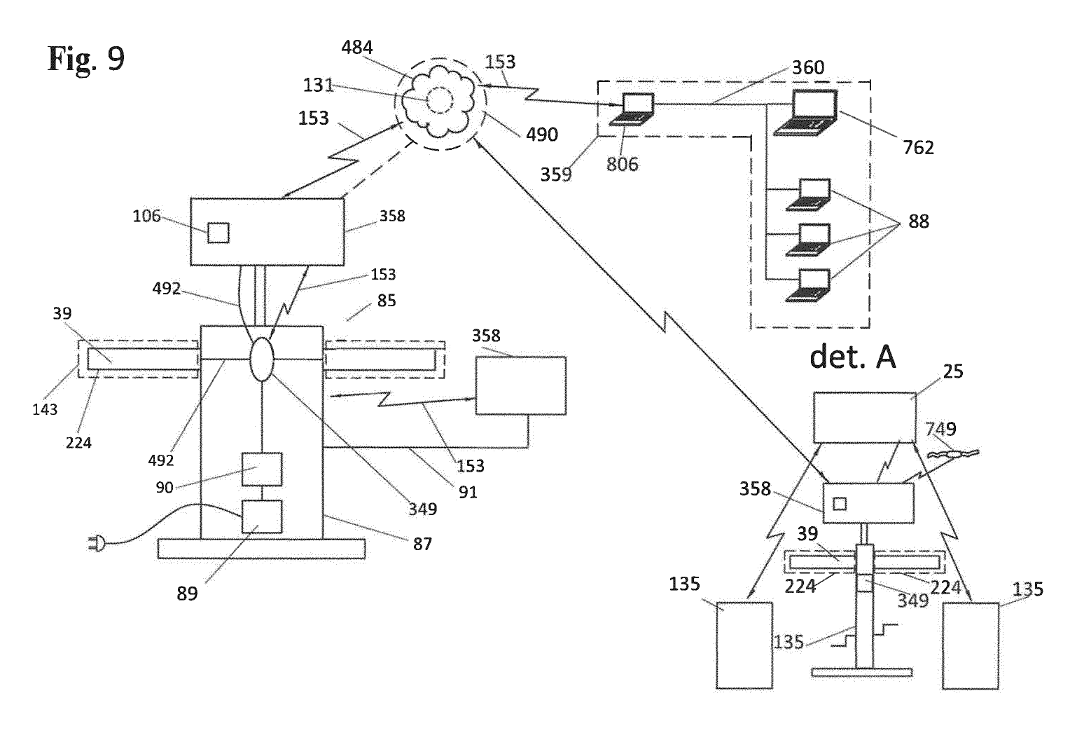

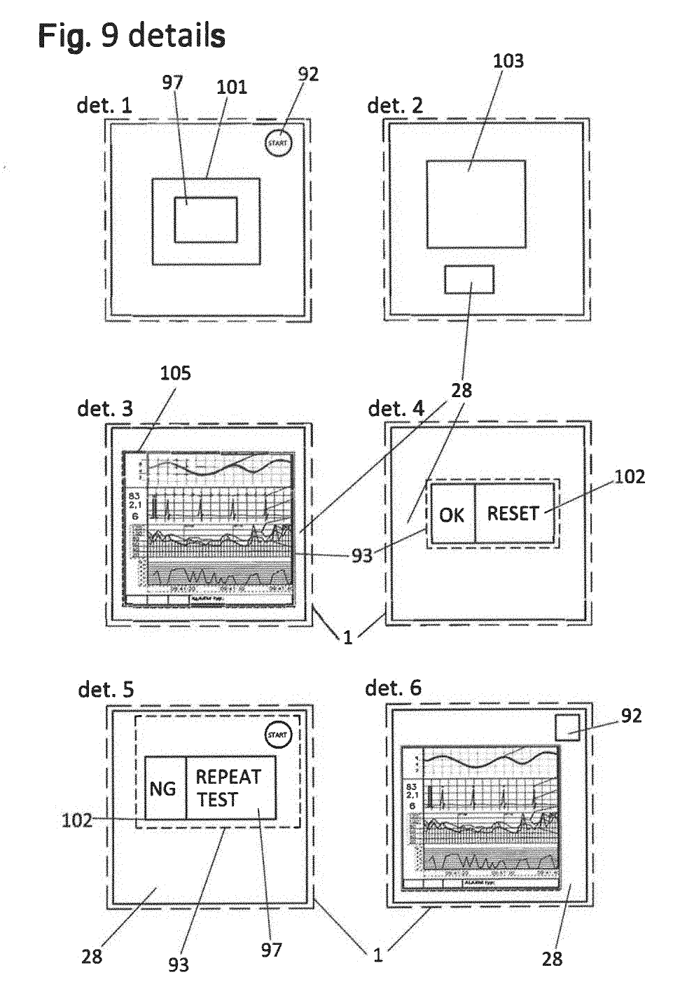

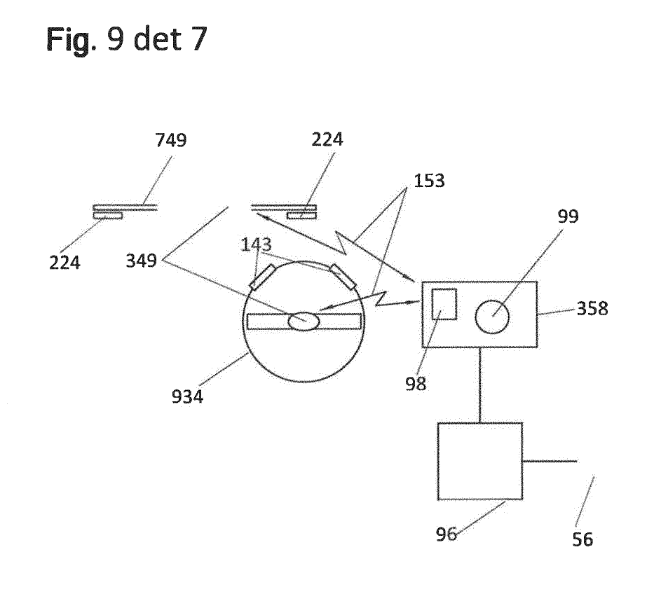

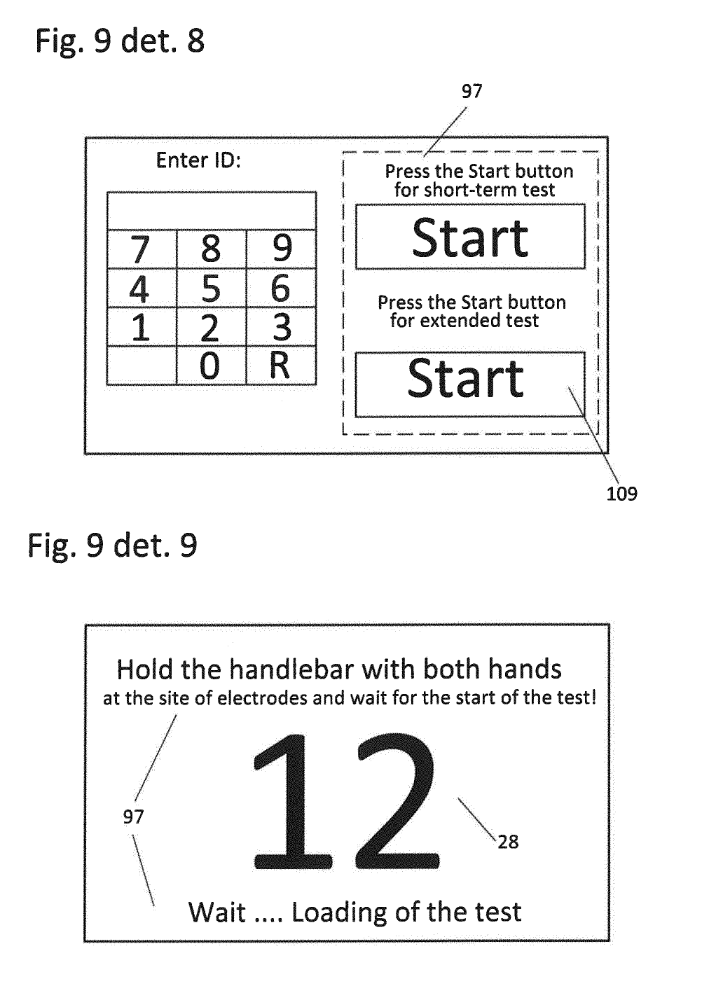

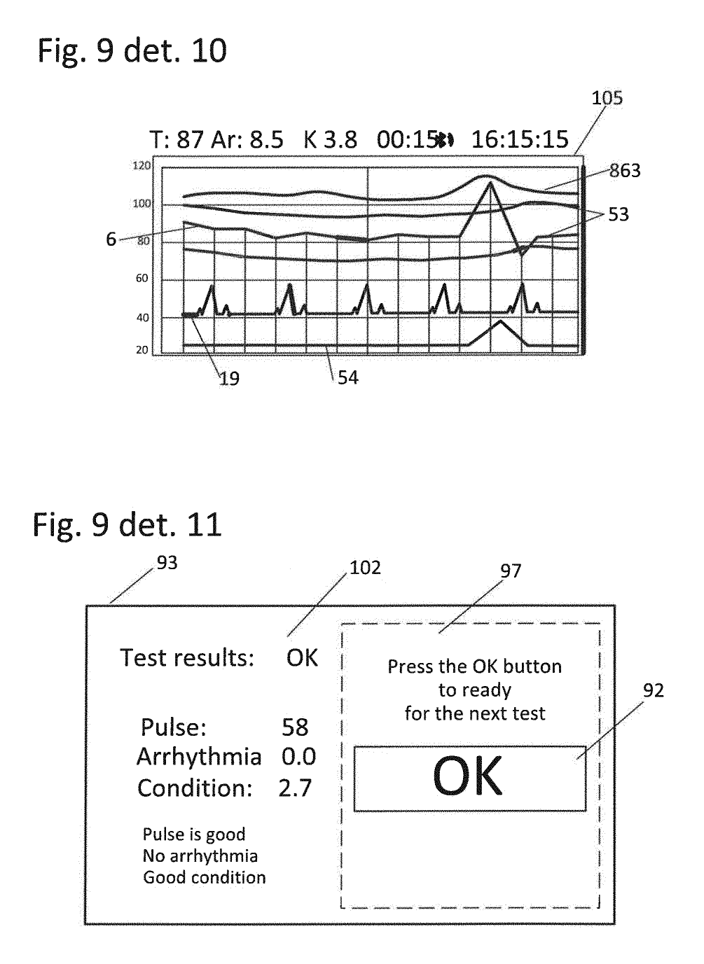

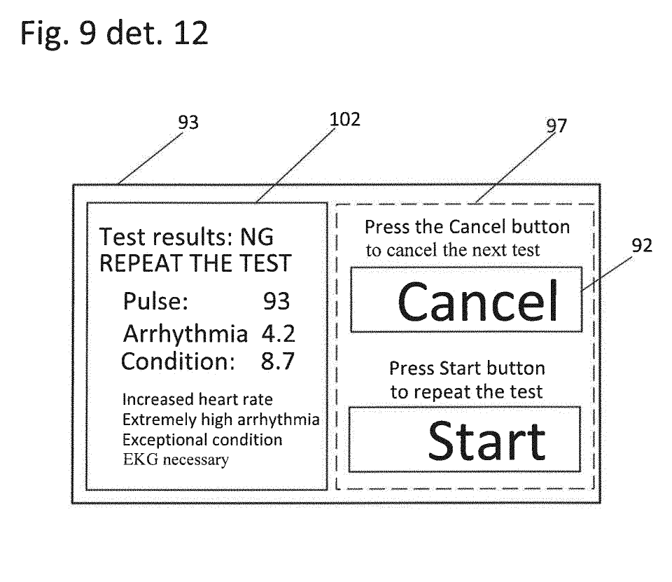

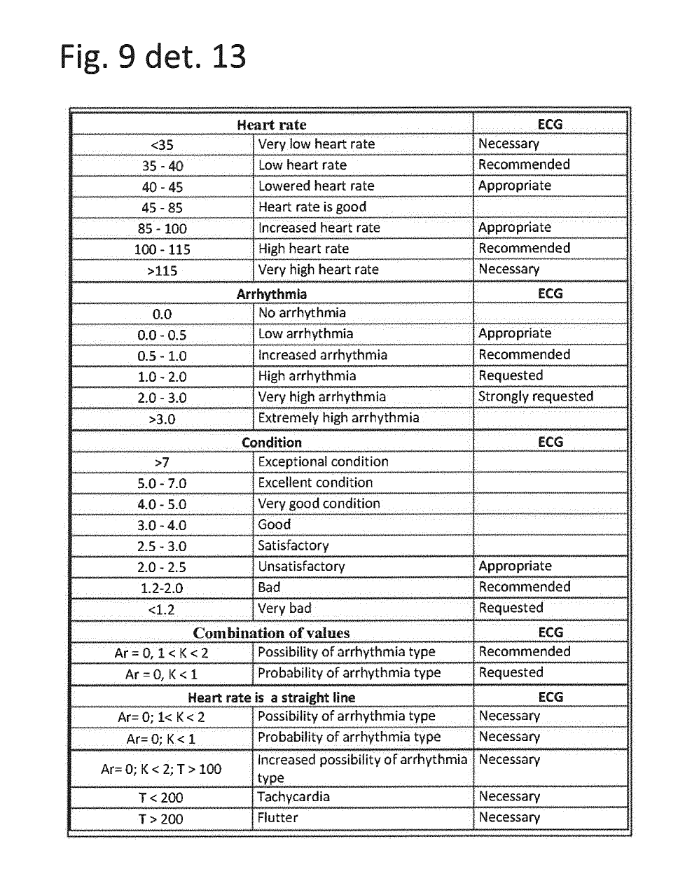

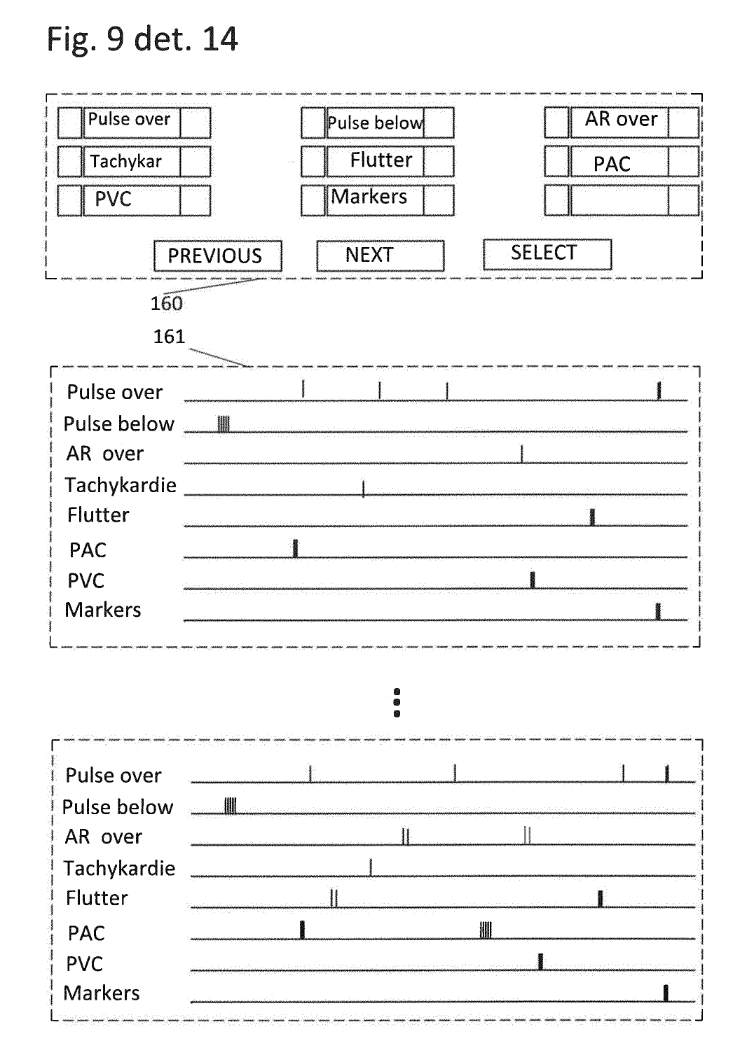

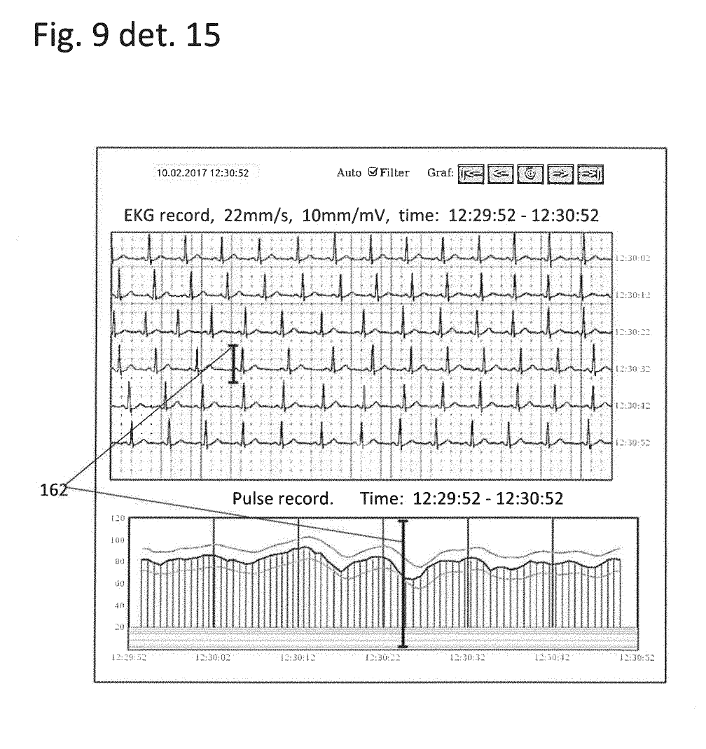

FIG. 9 shows a testing device 85 of immediate state of health, primarily the heart and psychical conditions, primarily to determine capability to drive a means of transport or to operate machinery or to perform a specific activity based on his/her job description which requires a specific capability, or for preventive current verification of his/her state of health, e.g. before a sports activity or during indisposition.

A testing device 85, adapted for mass testing of a larger number of monitored persons, advantageously consists of a stand 87 with electrodes 143 advantageously formed by contact areas 224, advantageously in the shape of a steering handlebar 39, on which a monitor 349 is mounted in a fixed way or detachably, advantageously powered advantageously by an accumulator 90 charged by a charger 89.

An output from a monitor 349, advantageously mounted removably on a steering handlebar, is connected using a wire link 492 and/or a wireless link 153 to a controlling and displaying module 358 on a stand 87 and/or to a remote one for a dispatcher.

The testing device is adapted for short tests of values and curves determined and calculated from heart rate, advantageously up to 1 minute, suitable also for mass testing, by putting both hands on electrodes when sitting/standing and the monitored person does not have to be at full rest.

Values derived from pulse are advantageously tested during an initial test and ECG is not advantageously tested, since to test pulse, a simpler and, therefore, a cheaper device suffices, and the test is also quicker and can be scanned from hands. If the initial test exceeds standards, ECG tests are advantageously performed at rest; the best way is in a prone position or, at least, in a sitting position using a monitor 349 mounted advantageously on a chest belt. At the same time, to initially determine physical condition of the monitored person, evaluation from pulses is mostly sufficient, and, since the test is simple and an ECG test is complicated, it is a preferred option.

The testing device can be, except positioning a monitor 349 and electrodes 143 on a stand 77, implemented with another advantageous design—with electrodes 143 advantageously in the form of contact areas 224 and the monitor 349 situated optionally, as required, on other devices or components described, for example in FIG. 4, primarily in det. 2C, 2D, 2F, 2G

A testing device 85 of a one-time test can be adapted for an individual test, e.g. in a means of transport or to a device controlled by the monitored person or at a different, suitable place, when electrodes 143, advantageously in the form of contact areas 224, are adapted for short-term putting on either fingers or palms of both hands simultaneously, for example on a steering wheel 934 or a sleeve 34 or on control levers of machines, or adapted for a one-time test 86, as shown in FIG. 4 det. 2C, 2D, 2F, 2G.

For a long-term testing, electrodes 143 connected to a monitor 349 are adapted for continuous contact with the body to scan cardiac signals, by placing on, for example, a chest belt 749, wristbands 485 or gluing on a chest.

On detail A FIG. 9, an alternative position of a steering handlebar 39 on a rotoped 135 is shown, where cardiac signals detected by a monitor 349 on a rotoped with contact areas 224 situated on the steering handlebar 36 or on a chest belt are displayed on a module 358 advantageously built-in to the rotoped 135.

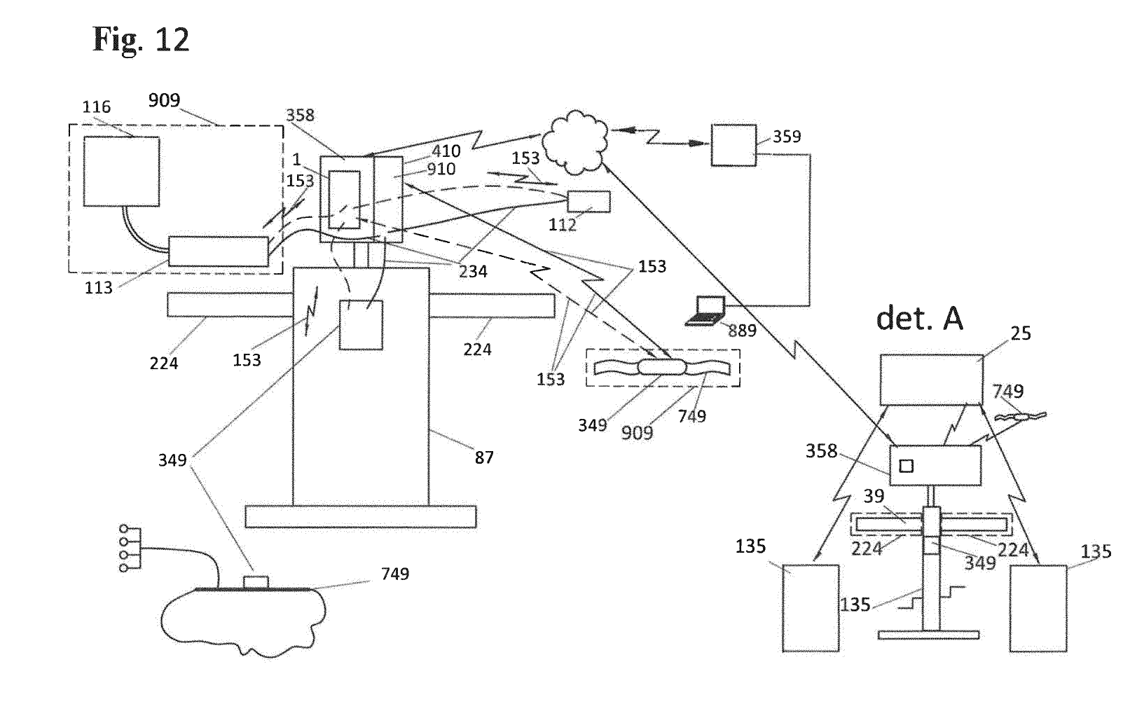

On a module 358, physical data of the monitored person pedaling on a rotoped are displayed, for example burnt calories, rpm, time, advantageously on a shared display with data of cardiac signals and/or state of health described in FIG. 12.

FIG. 9 det. 1 shows a display element 1 of a monitor advantageously formed by a display 28 in a default phase of an initial test with initial display 101 with a prompt to start the test by pressing the push-button 99 “Start” and putting both hands on electrodes 143 advantageously formed by contact areas 224.

FIG. 9 det. 2 shows how, when the push-button 99 “Start” has been pressed for an adjustable time necessary to stabilize curves of pulse and other values, a display element 1 advantageously shows waiting display 103 with “count-down” display 28 of the time remaining to start the test.

FIG. 9 det. 3 shows testing display 105 with values and/or curves of optional pulse and/or ECG and/or arrhythmia and/or variability, or testing display 105 optionally is not displayed and wait display 103 is displayed until the test is finished and changes directly to resulting display 93 shown in det. 4, 5.

FIG. 9 det. 4 shows how after an adjustable time period, advantageously up to 1 minute to enable a large number of monitored persons to undergo the test, resulting display 93 of the test is displayed, summarizing OK good status with a test result 102, which enables to drive a means of transport or operate a machine which indicates a good state of health.

FIG. 9 det. 5 a bad condition “NO GOOD” (N.G.), requiring a specific action, e.g. verifying an ECG test as described on a display element 1 before the required activity is performed, for example driving a means of transport.

Advantageously for an N.G. state, the test is advantageously repeated for a longer time period to eliminate errors of the initial test, advantageously on a different testing device 85; the tested person is prompted to do so by an instruction 97 to repeat the test on resulting display 93 of the results. During the second test performed on the same testing device 85, a push-button 92 of the second test appears within the resulting display 93 and the monitored person is prompted to push the button by the instruction 97. If the result of a repeated test is NG, one of recommended subsequent actions is to undergo an ECG test, on a testing monitor 349 on a stand, if it is designed this way, or on other monitor 349, advantageously with electrodes attached to a chest for better accuracy and advantageously equipped with electrodes for 12-lead ECG, which is evaluated automatically in a module 358 and/or is evaluated by a trained employee and/or is evaluated by health-care staff advantageously a physician at the same place where the testing device is. Or, cardiac signals scanned advantageously using a monitor 349 advantageously by a module 358 are sent using a long-distance link 94 advantageously via a WiFi network 131 or a mobile operator 484 network to an evaluation block 359 advantageously to an evaluation block 359 advantageously to a server 806 and/or remote participants for evaluation purposes. This is advantageously performed automatically on a server 806 and the result is sent in the form of data to a display element 1 of a module 358 and/or is assessed by an expert health-care staff which communicates with a dispatcher advantageously situated at the place of the test and/or the monitored person, by phone or as data.

FIG. 9 det. 6 shows how, during long-term testing, the result of the current test is displayed by pressing a push-button 92 advantageously labelled “RESULT” on a display element 1 on a module 358 which shows continuously measured curve values. During long-term testing, it scans cardiac signals from electrodes 143 attached permanently to the body of the monitored person, for example on a chest belt 749 with a monitor, and the processed data are displayed and/or stored in a memory medium 106, for example a module 358. From this data, a result of the current test displayed in FIG. 9 det. 4, 5, 11, 12 is processed and displayed based on a request for results, advantageously by a push-button 92 in a module 358.

FIG. 9 det. 7 shows how during short-term testing the test is advantageously started automatically by putting on hands on electrodes 143 advantageously formed by contact areas 224, for example on a steering wheel 934, which is detected by a detector 98 of cardiac signals advantageously included in a monitor 349. This is advantageous for testing during a drive, since it is not necessary to push the START button.

Or, the test is advantageously launched by a voice command detected by a voice coder 96, with a microphone 56 connected to a module 358, which is advantageous primarily during driving or when controlling a machine.

FIG. 9 det. 8 shows a detailed initial display 101, a display element 1, advantageously on a module 358, which is advantageously displayed during initializing a preparatory stage for short-term testing of physical condition using a corresponding command entered to, tier example, a module 358, which displays an instruction 97, for example “Enter ID” and “To test physical condition, press “START””.

Instead of pressing the Start button, the test is advantageously started automatically after grasping electrodes 143, for example on a steering wheel 934, by both hands, which is detected by a detector 98 of cardiac signals, advantageously in a monitor 349, or by a voice command detected by a voice coder 96, as described in det. 7—an instruction 97 on initial display 101 prompts to do so. A button 109 advantageously appears also on initial display 101 for extended testing, for example a doubled test where each tap on the button doubles the test duration and longer pressing the button changes the test to a long-term one.

FIG. 9 det. 9 shows a detailed example of waiting display 103 after a button 92 has been pressed on initial display in det. 8 or after an alternative test start described in det. 7. On waiting display 103, an instruction 97 is displayed, for example, “Hold the steering handlebar by both hands where the electrodes are positioned and wait until the test starts”. Moreover, there is advantageously an instruction 97, for example, “Wait, test parameters are being adjusted”. Before the test starts, a count-down tinier is advantageously displayed.

FIG. 9 det. 10 shows how after test initialization, for example using a push-button 92, progress testing display 105 is advantageously displayed on a display element 1 after wait display 103 is finished, where the measurement progress is displayed advantageously with pulse curves 6 and/or values with regular pulse limits 53, arrhythmia 54 and/or variability 863 and/or ECG 19. This screen is advantageously displayed with adjustable delay after a push-button 92 has been pressed, during which wait display 103 is displayed to hide distorted curves during a stabilization period of a testing device 85 caused, for example, by noise, after by pressing hands on electrodes 950.

FIG. 9 det. 11 shows how after the test, advantageously up to 1 minute, resulting display 93 with a test result 102 is shown, for example “Test OK”. In an instruction 97, a notice 108 is advantageously displayed to conclude the test, for example “Press OK to prepare for another test”.

FIG. 9 det. 12 shows a negative result “NO GOOD”, abbreviated as N.G. On resulting display 93, an instruction 97 is advantageously displayed to repeat the test, advantageously for a longer time period, for example up to 3 minutes, to eliminate a testing error. If the test is N.G. once more and if ECG was not tested in the initial or repeated test, this screen 93 of the repeated test shows, apart from results, a recommendation to perform and evaluate an ECG test.

ECG is advantageously not tested during an initial test since a simpler and cheaper device suffices to perform a pulse test, and the test is also faster and it can be scanned from hands in, for example, a standing position when the monitored person is not required to be at absolute rest which is necessary to monitor ECG. At the same time, to initially determine physical condition of the monitored person, evaluation from pulses is mostly sufficient, and, since the test is simple and an ECG test is complicated, it is a preferred option.

An ECG test is advantageously performed on other monitor 349 with ECG than the one the initial physical condition test was performed on. This enables to use a cheaper monitor 349 without ECG for the initial test and, moreover, during mass measuring the monitor 349 is not occupied to the degree it would be occupied if the monitor were also used for ECG which takes more time than a pulse test.

For ECG testing purposes, the monitored person is introduced to as calm state as possible, in a prone position or, at least, in a sitting position, and a monitor 349 is connected to electrodes 143 advantageously in the form of contact areas 224 to scan cardiac signals from a chest; on a chest belt 749, for example, or on electrodes 143 glued to the chest.

To enable performing the test without taking of clothing, for example a shirt, a chest belt is advantageously applied as shown in FIG. 8.

The result of initial measurement from the pulse and/or a repeated test and/or from ECG advantageously sends data to an evaluation block 359, advantageously to a server 806, where it is evaluated automatically and the result is sent back as data to a module 358 of the monitored person or to dispatcher's PC. Or, an expert health-care staff like a physician evaluates it and uses a data message or phone to send the result to the dispatcher who arranges mass testing or to the monitored person.

For ECG testing purposes, a monitor 349 in the testing device is advantageously equipped with more electrodes, up to 10 for scanning up to 12-lead ECG. Alternatively, other ECG device is used which enables to send the result to an evaluation block 359, a server 806 and/or to remote participants 88.

FIG. 9 det. 13 shows a table to display on a display element 1 from which results are retrieved based on measured values advantageously as numbers or words if the test result is negative. A test result 102 evaluates advantageously pulse, arrhythmia, physical condition, which is advantageously a variability value calculated from the pulse and a combination of the values. The initial test is performed advantageously using a simplified method using the pulse and arrhythmia level or also variability levels, with subsequent ECG testing for negative results, or using the full method with ECG. During the initial test with a simplified procedure, sinusoidal curve is evaluated which means the OK result for zero arrhythmia level. Or arrhythmia occurs when the arrhythmia level is non-zero which indicates a negative NG result. If arrhythmia occurs, its arrhythmia level is evaluated, from insignificant to high. 10% of irregular pulses from the entire number of pulses is considered as one arrhythmia level. Therefore, 1st level corresponds to 10%, 5th level corresponds to 50%. Based on this, the highest, 10th level corresponds to only a theoretical value of 100%. Heart rate levels, i.e. number of heart beats per minute, are advantageously expressed verbally in the following manner: <35 very low pulse, 35-40=low pulse, 40-45=lowered pulse, 45-85=normal pulse, 85-100=increased pulse, 100-115=high pulse, >115=very high pulse.

Arrhythmia levels are advantageously expressed verbally the following way: Ar=0 none, 0<Ar≤0.5 low, 0.5<Ar≤1 increased, 1<Ar≤2 high, 2<Ar≤3 very high, 3<Ar extremely high arrhythmia.

Physical condition levels, advantageously resulting from variability, are advantageously expressed by words the following way: >7=exceptional, 5.0-7.0=excellent, 4.0-5.0=very good, 3.0-4.0=good, 2.5-3.0=satisfactory, 2.0-2.5=unsatisfactory, 1.2=poor, <1.2 very poor.

Combination of the above mentioned values is advantageously expressed by words the following manner:

1. Ar=0; 1<K<2 Possibility of arrhythmia of the type (see 4, 5)

2. Ar=0; K<1 Likelihood of arrhythmia of the type (see 4, 5)

3. Ar=0; K<2; T>100 Increased likelihood of arrhythmia of the type (see 4,5)

4. T<200 Tachycardia

5. T>200 Flutter

Moreover, individual extrasystoles and bursts of extrasystoles, pulse drop-outs and cardiac arrest are advantageously evaluated from the curve of heart rate.

13% of irregular pulses is allowed to consider the curve sinusoidal, and regular pulses are defined by limit curves 17 of a regular pulse in FIG. 7.

A totally regular pulse with 0 arrhythmias does not mean the pulse is sinusoidal, but it has to be tested for arrhythmia of the tachycardia type when the pulse is under 200 pulses/min or flutter when it is higher.

Therefore, not just arrhythmia with allowed tolerance is tested but also pulse which, if not changing, i.e. the pulse curve is a straight line for a specific adjustable number of pulses, means a suspected arrhythmia which is confirmed by low variability (less than 2) measured for a longer time period than to determine that pulse is a straight line.

Therefore, if variability is lower than 2 in the test results on a display element 1, it is recommended to repeat the test and, if the result is confirmed, primarily if the pulse is a straight line, to perform an ECG test.

Moreover, pulse higher than 85 is evaluated for which it is recommended to repeat the test after a specific time spent at rest.

If it is confirmed, an ECG test is recommended like for too low pulse, advantageously lower than 45, or pulse drop-outs.

During pulse testing using a simplified method using pulse and arrhythmia levels and variability, their combinations are evaluated based on the definition in the table in det. 12. As for variability, a variability value lower than 2.5 is evaluated as NG since it indicates poorer physical condition with a risk of microsleep during driving means of transport or operating machinery. This risk increases when the variability level decreases. Verbal evaluation of the level is, for pulse, arrhythmia or variability results, defined in the table in det. 12. For NG results, it is recommended to perform other test after relaxation due to low variability. When a NG result is confirmed, an ECG test is recommended.

For ECG, QRS complex, A-V block, PAC, PUC, AF, FLUTTER, TACHYCADIA and other pathological states are evaluated, besides states of the initial test. Evaluation is performed preliminary automatically in a monitor 349 or in a module 358 or, more precisely, in an evaluation block 359 advantageously on a PC on a server 806 or on PCs of remote participants and advantageously sent back to the module 358 of the monitored person. Or, evaluation is performed manually by expert health-care staff, advantageously by a physician, and is sent to a module 358 as data or is reported to the monitored person or dispatcher who performs mass tests, by phone or email. When 1st test is negative, the next, 2nd test is advantageously performed using a push-button 92.

For long-term or continuous tests, automatic evaluation of NG states is advantageously set in a monitor 349 and/or module 357, 358 and/or evaluation block 359 with alarm initialization if they occur. To cut costs for transferring data, data are sent in bulks to an evaluation block 359, or just the states exceeding predefined limits or only from time to time based on the monitored person's command, or are not transferred and are stored in a memory medium 106 in a monitor 349 and/or a module 357, 358, advantageously in the form of an SD card whose content is transferred to a PC. To evaluate data from the PC, they can be transferred via the Internet which is cheaper than using a mobile operator. Or they are sent from a monitor 349 or a module 357, 358 through a local WiFi network.

Evaluation for various activities is advantageously performed from tests. For example, for driving means of transport, a notice of danger of microsleep is issued for physical condition <2.5, using reflection; for sport, it is a possibility of overtraining when physical condition is poor, long after training it is generally danger of stroke for arrhythmia.

FIG. 9 det. 14 shows a result of evaluation of a long-time record of the “Holter” type. This automatic evaluation can be requested using the “HOLTER” button, from a several-minute record to a many-month long record, and a result of a specific record period or more periods which can be selected from the record can be advantageously requested.

If the requested period's length is different than 24 hours, it is possible to ask to recalculate the results to 24-hour-long record to get standard results for 24-hour-long Holter.

The result is processed in a monitor 349 or in modules 357, 358 or 359 and is displayed in a display element 1 or is printed. A monitor 349 positioned in a testing device 85 enables to perform a one-time test and, when completed with accessories which enable to move the monitor 349 for long-term testing to, for example, a chest belt, the monitor with accessories is adapted for automatic processing of long-term tests, advantageously of the Holter type.

The figure shows an example how a long-term record of cardiac signals of the monitored person is displayed, i.e. records for 8 out of the entire set of optional parameters for monitoring.

These are: exceeding an adjustable upper limit of the heart rate named “Heart rate above”, exceeding the minimum heart rate named “Heart rate below”, arrhythmia above an adjustable value named “AR above”, arrhythmia below an adjustable value named “AR below”, tachycardia occurrence named “Tachycar”, flutter occurrence named “Flutter”, time marks specified by the monitored person in times when he/she did not feel comfortably, markers and early “PAC” atrial contractions. For applicable parameters, the operator specifies monitoring limits before measurement, for example maximum/minimum values. First, the operator uses a selection field 160 to select parameters to be displayed in each of fields 161 of curve displaying. Number of these fields on the display is optional, based on the number of selected parameters and planned length of the record to achieve maximum utilization of the display. Two such fields are shown in the example, displaying 8 selected parameters.

Time is shown on the X-axis, occurrence of the monitored phenomenon, parameter is shown on the Y-axis using a short line. Since it is a long-term record where one display contains a time range up to 1 month, occurrences during the time period can blend to a single thick line or a rectangle. This indicates that the monitored phenomena occurred in this time period. The operator can use an electronic time-magnifying glass and change the scale as required to separate the blended records. By placing the magnifying glass over a specific spot within the record and selecting the scale, for example moving from a month to a week, the monitored spot on the time axis is transferred to one field and records in the same time scale are displayed in remaining fields; however, they precede or follow the selected spot so that all fields are logically tied. Therefore, the operator's advantage is that he/she can quickly determine whether and when the monitored phenomena occurred and, in turn, can display a precise time of an individual occurrence of the phenomenon in a few steps. This moment, he/she can switch to a record of ECG curves and pulse of this moment and its adjoining regions.

FIG. 9 det. 15 shows how ECG is displayed after switching from display described in FIG. 9 det. 14. An advantage is to display six ECG curves, one under another, each following a preceding one, advantageously the first at the bottom, the last on top. A heart rate curve corresponds to these curves and six sections corresponding to its ECG curves are highlighted within the curve, the earliest section on the right, the latest on the left. The corresponding spots in both records are marked using a cursor 162.

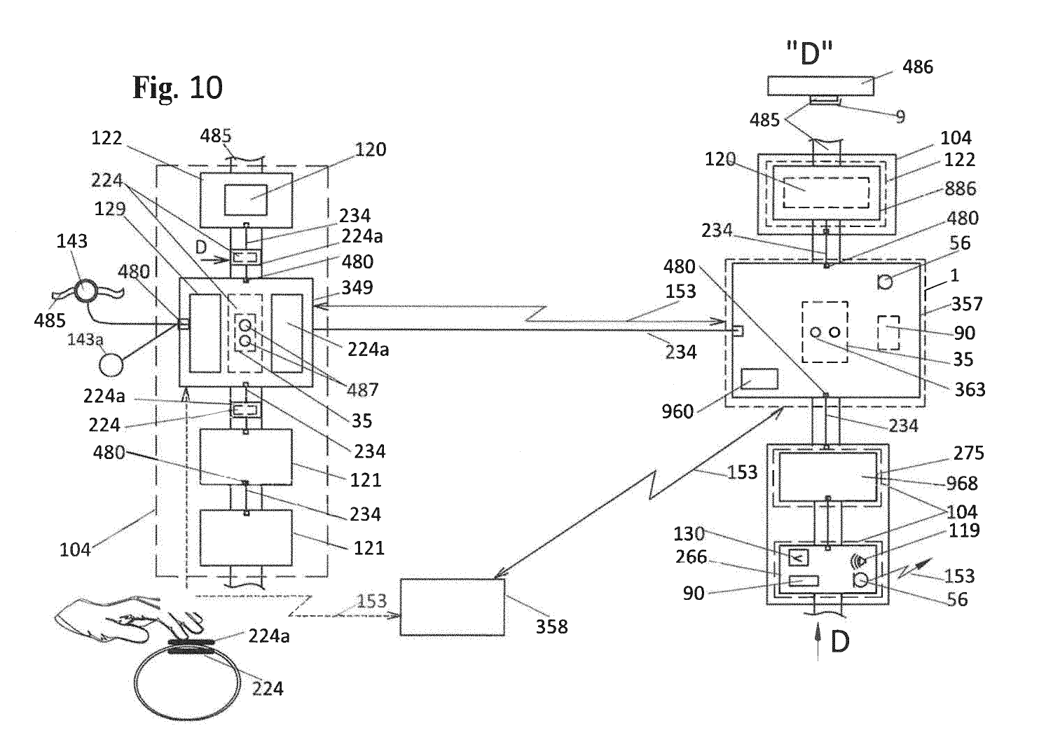

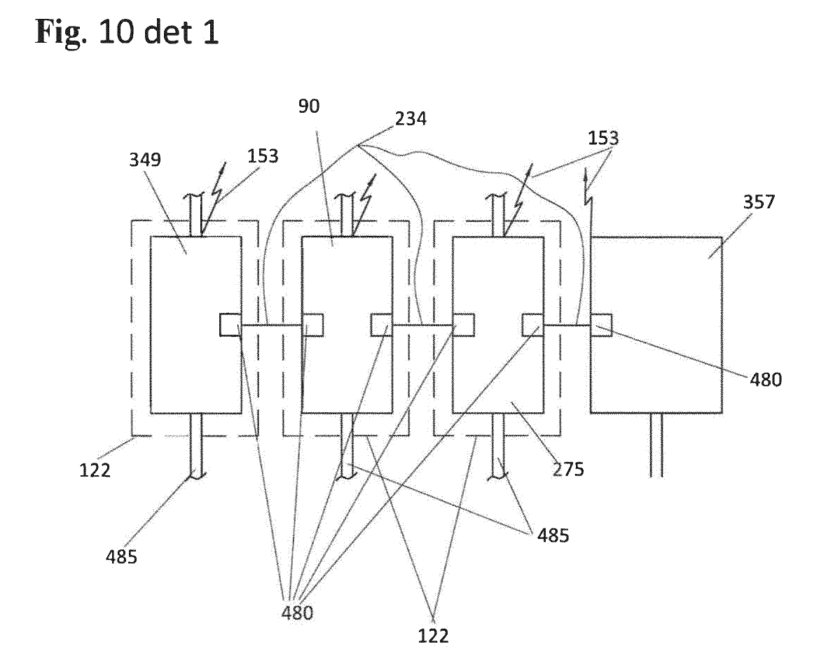

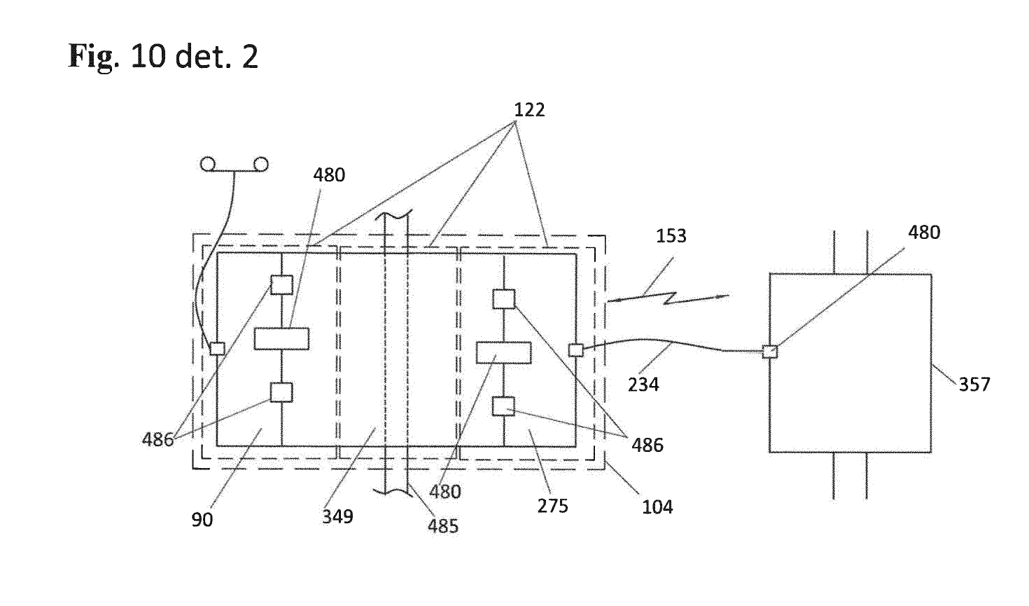

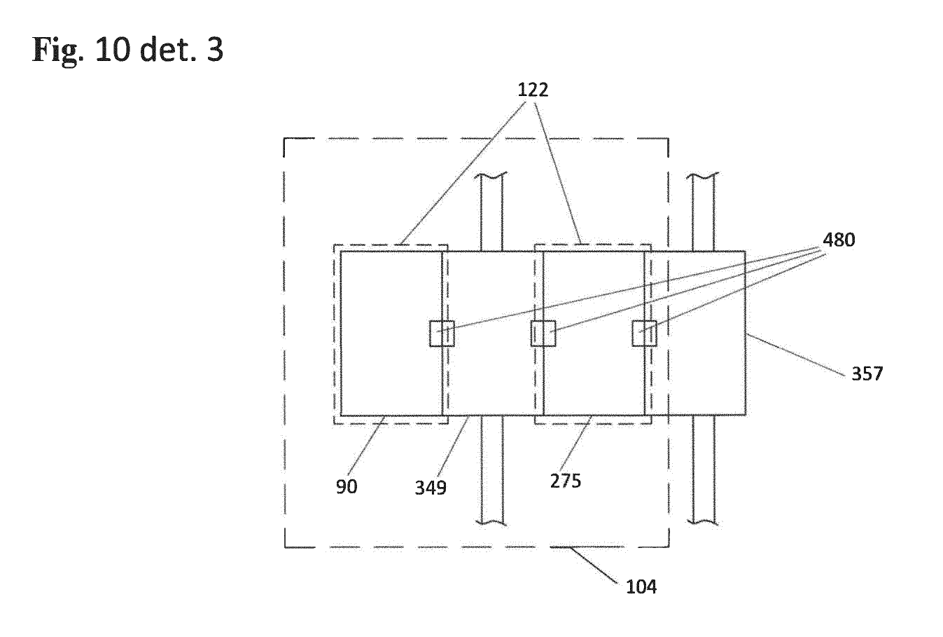

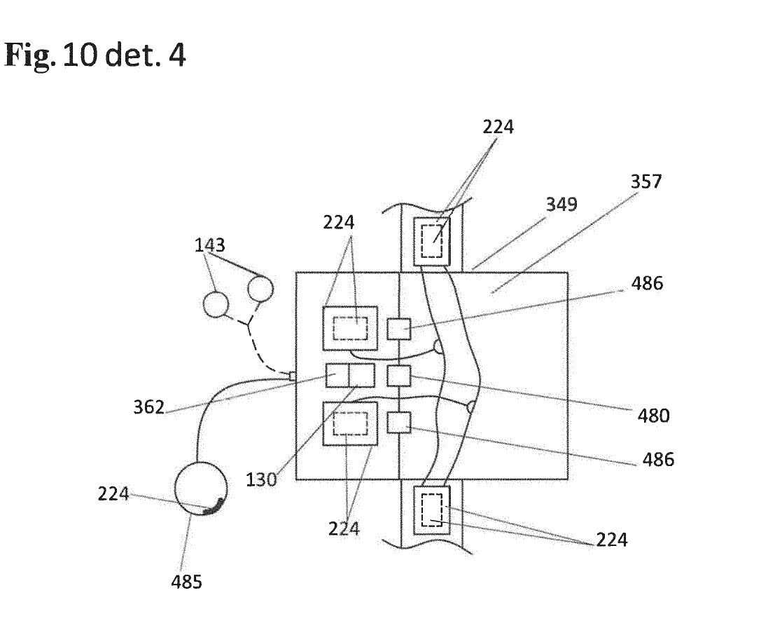

FIG. 10 shows the attachment device 104 of the controlling and displaying operative module 357 preferably comprising the block 121 of additional units preferably positioned on one or more wristbands 485, 43. The monitor 349 is preferably attached in a detachable manner on the fastening element 35, preferably formed by the snap fasteners 487, on the wristband 485 on the wrist; it is preferably connected by the short cable 234 via the connector 480 or via the wireless link 153 to the operative module 357 preferably positioned in a detachable manner on the same or another wristband 485 preferably formed by a small mobile phone by approximately 1 cm narrower than the wrist for comfortable and aesthetic wearing.

The monitor 349 has, preferably on the part applied on the arm or wrist, the contact area 224 preferably forming the ECG electrode 143 from first arm, and on the upper part the other contact area 224 for a finger preferably forming the ECG electrode 143 of the second arm. Signals of heart rate and/or ECG for the processing are taken preferably from these electrodes 143.

Alternatively, the electrode 143 of the second arm is positioned instead of on the monitor 349 on the wristband 485 on the second arm connected by the short cable 234 via the connector 480 to the monitor 349, or this electrode 143 of the second arm is glued and positioned on the chest, preferably connected in the place suitable for heart signal sensing on the second arm and into the connector 80 using a wire, whereby these electrodes are extensible by additional electrodes for the detection of up to a 12-lead ECG, in which case preferably without using the contact areas 224 on the monitor. Alternatively, the contact areas 224, instead of on the monitor 349, are placed from below the wristband to make a contact with the wrist and from above of the wristband to make a contact with the fingers of the second arm, preferably one above the other, so that two fingers, preferably the thumb and index finger of the second arm, pressed against the upper contact areas 224 while also pressing down the bottom contacts on the forearm or wrist.

The monitor 349 is preferably equipped with the accumulator 90, preferably adaptable to power the operative module 357 via the short cable 234, to which they are connected, preferably for the purpose of data communication as well. The attachment devices 104 consisting of the additional units 121, such as other accumulators, detectors, communication units 275, are preferably fastened, preferably in a detachable manner, on the wristband 485 of the monitor 349 or on other wristbands 485 next to the monitor 349.

The operative module 357 on the wristband 485 with the display element 1, with a control unit and preferably equipped with the optical front-end 363 for sensing of heart rate and oxygen content in blood by the radiography of the skin on the forearm, is preferably equipped with the additional attachment devices 104 placeable externally on the wristband 485, preferably in a detachable manner on the spring clamp 9, or it has them placed internally, such as the communication unit 275 preferably formed by the 5.3 kHz and/or “Bluetooth” and/or ANT receiver, and the attachment devices formed for example by the additional accumulator 120, preferably embedded in a removable manner in the housing 886 for the accumulator, and/or in addition for example the voice unit 266 preferably attached in a detachable manner on the wristband 485 preferably using the spring clamp 9.

The operative module 357, preferably of a smaller size, communicates using the wireless link 153 with the module 358, preferably of a larger size and preferably consisting of a mobile phone or tablet, and these modules are adaptable to mutual reception and display of data concerning in particular telephone calls or SMS preferably realized using a SIM card in the module 358 and preferably initialized in the operative module 357. The operative module 357 is preferably adaptable to receive telephone calls or SMS coming into the module 357 and/or the module 358 and operate calls in the “hands free” mode or by putting it to the ear after detaching it from the wristband 485 or using the voice unit 266, adapted for putting to the ear attached, preferably in a detachable manner, to the operative module 357 or its wristband and communicating with the operative module via the wireless link 151. It includes an accumulator, preferably replaceable, the microphone 56, speaker 47 and the control unit 130 with an amplifier. Voice detection is preferably performed using the microphone 56 integrated in the operative module 357 so that the transmission of sound to the voice unit is one-way only to the speaker, and for wireless communication preferably one-way radio signal with a limited range, which does not require coded connection establishment and is energy efficient and provides reception, is used. In such a case, the microphone is preferably placed in the module 357. Alternatively, it is possible to use for communication the detachable module 357 which is put to the ear after being detached. Preferably, the fastening element 35 for fastening and for removal of the module 357 is preferably equipped with a connector allowing a detachable connection of connected units. The module 357 can be adapted to operate independently without the module 358. In such a case it is preferably equipped with a SIM card for telephone operation.

The operative module 357 is preferably adaptable for the connection of other external attachment devices, such as the temperature, pulse and oxygen in blood detector 112 preferably attached to a finger, or the pressure meter 113 preferably attached to the arm or wrist.

FIG. 10 det. 1 shows the attachment device 104 preferably consisting of the additional units 122 attached preferably to the independent wristbands 485 connected by the short cables 234 or wireless links 153 to the operative module 357.

From pulse detected like this, preferably the operative module 357 or the module 358 processes them to generate pulse values and/or curves, arrhythmia, variability, or the operative module 357 is preferably adaptable to the processing of pulse sensed for example by the monitor 142.

FIG. 10 det. 2 shows a compact embodiment of the attachment device 104, where the additional units 122 are connected to one another in a detachable manner using the mounting element 486 and the connector 480 which is used also for the connection of the attachment device with the operative module 357 using the short cable 234 or the wireless link 153. Alternatively, the attachment device 104 is connected to the operative module 357 directly by the connector 480 without the short cable and using the mounting elements 486 as provided in det. 3.

FIG. 10 det. 4 shows the monitor 349 integrated in the operative module 357 with the contact areas 224 on the top and bottom sides of the monitor 349 to make a contact with the forearm of the first arm and the fingers of the second arm, which are preferably replaceable by the contact area 224 on the wristband of the second arm or the glued electrode 143 on the chest, or the contact areas are replaceable by two glued electrodes 143 on the chest. Alternatively, the contact areas 224 are placed from below and from above on the wristband to make a contact with the forearm of the first arm and the fingers of the second arm. The contact areas 224, preferably the electrodes 143 are connected by short cables to the ECG front-end 362 and to the control unit 365 of the monitor 349 for processing and from here data is transmitted to the operative module 357 preferably for display. The monitor 349 is alternatively connected in a detachable manner to the operative module 357 using the connector 480 and the mounting element 486, which is represented by a dashed line.

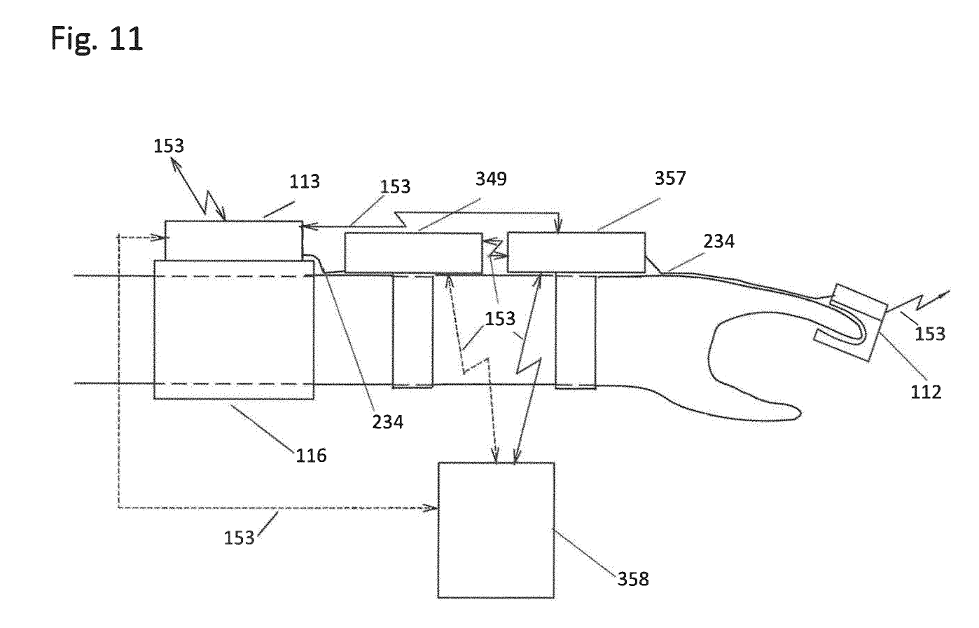

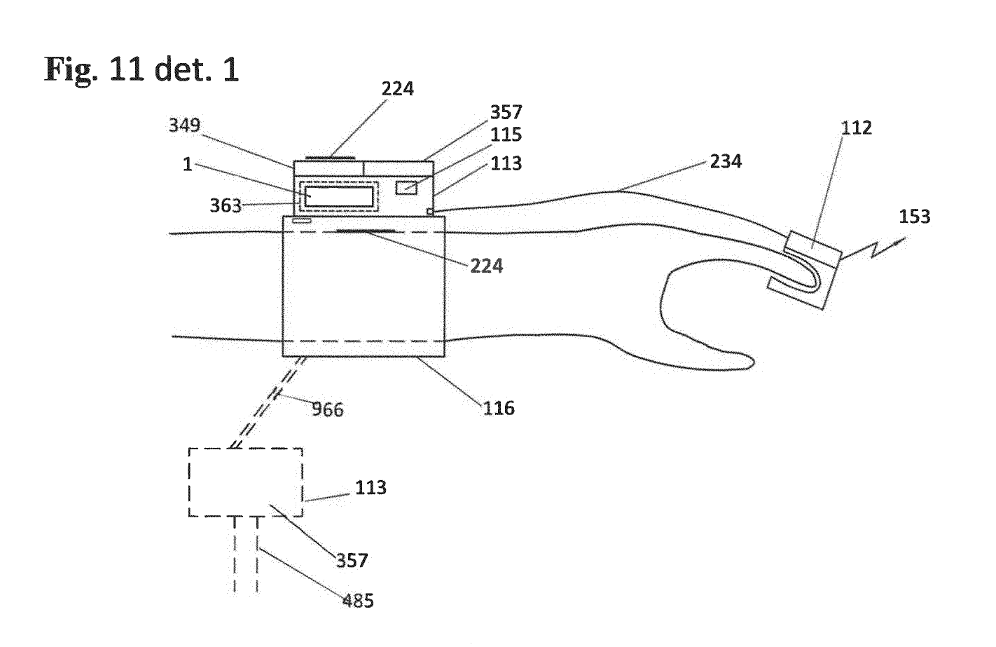

FIG. 11 shows the pressure meter 113 and oxygen in blood detector 112 which are connected by the short cable 234 or the wireless link 153 to the operative module 357 which is connected preferably to the monitor 349 for ECG or pulse data. In particular, if the monitor 349 is not used, preferably the operative module 357 receives the heart rate values alternatively from the detector 112 instead of from the monitor 349.

Alternatively, preferably the pressure monitor 114 detects pulse from pressure in the pressure sensing sleeve 116 sensing the pressure which changes in the rhythm of the pulse, where a decrease in pressure in the sleeve 116 is preferably decelerated to acquire a longer record of pulse, or, upon reaching the values of the bottom pressure, the pressure in the sleeve 116 is increased again up to the level where pulse is measurable to attain a longer record of the pulse values. Data from the pressure meter is transmitted to the operative module 357 on the arm or wrist using the short cable 234 or the wireless link 153.

From pulse detected like this, preferably the operative module 357 or the module 358, processes the data to generate the pulse values and/or curves, arrhythmia, variability, or the operative module 357 is preferably adaptable to the processing of pulse detected for example by the monitor 349. The monitor 349 and/or the module 357 and/or the pressure meter 113 are preferably connectable to the module 358 to display data or results of measurements. From the module 357, 358 the pressure meter is controllable. Additional meters sensing medical data from the body of the monitored person are connected similarly.

FIG. 11 det. 1 shows the pressure meter 113 preferably placed on the sleeve 116 sensing pressure on the forearm or wrist, or the pressure meter 113 is placed on the wrist separately from the sleeve and is connectable by a tube. On it, preferably in a detachable manner, the monitor 349 is placed or the monitor 349 is integrated in the pressure meter. The ECG electrode 143 from first arm is positioned from below on the sleeve 116 to make a contact with the first arm and with the other contact area 224 preferably on the surface of the monitor 349, or from above on the sleeve 116, preferably forming the electrode 143 for the fingers of the second arm to detect preferably pulse or ECG signals, these electrodes 143 are connected to the monitor 349 to process cardiac signals. The control unit 115 of the pressure meter processes pressure data and sends it to the operative module 357 where also pulse and ECG data from the monitor 349 is transmitted. Such data can be preferably displayed also on the display element 1 of the pressure meter 113. In the operative module 357 the data is processed and saved in the memory and/or displayed on the display element 1 and/or transmitted to the module 358 and/or the block 359 for evaluation. Preferably the operative module 357 also receives data from the oxygen in blood detector 112 and the body temperature sensor 751 placed preferably on the finger, connected by the short cable 234 or the wireless link 153. Preferably during the monitoring of arrhythmia or extrasystoles or failing pulse the cardiac rhythm is transmitted by the 5.3 kHz induction link which is energy-efficient, and in the case of deviations in quantities subject to monitoring from the adjustable standard, full ECG data will start to be transmitted via a link which is able to transmit it, such as Bluetooth which is energy intensive. Preferably Bluetooth Low Energy, or another technology energy-efficient is preferably used in the case of the transmission of information on pulse and its energy intensity is only increased no sooner than upon the start of the data stream transition necessary for the ECG transmission. Evaluation from the pulse curve to discover pathologies, such as arrhythmia, instead of from ECG, for the representation of such phenomena, is not only energy-efficient but also has the advantage resting in the verifiability for a longer period of time. The ECG curve can only be transmitted in moments when the monitored data exceeds the adjustable limits. The ECG curve is not preferably transmitted but saved continuously in the memory of the monitor 349 to be transmitted, if necessary, not only from the moment when a deviation in the pulse curve was discovered but for any optional period of time before. Saving data is preferably performed on a portable memory medium, preferably an SD card, to allow the data to be transferred not only in electronic form but also by inserting the SD card into a selected device. In such a manner the monitor 349 operates as a Holter with a long record available to long-term monitoring for up to several months.

FIG. 12 shows the connection via the short cable 234 or the wireless link 153 of the pressure meter 113, oxygen in blood detector 112 and monitor 349 either directly or into the module 358, preferably positioned on the stand 87 or the Rotoped 135. From the module 358 data is transmitted preferably to the evaluation block 359 and the evaluated data is preferably transmitted back to the system operator's PC preferably localized at the stand or the person being monitored. Attachment of the contact areas and described units is preferably adaptable to a different suitable arrangement than on the stand 87.

The stand 87 or the Rotoped 135 with the chest belt 749 with the monitor 349, either autonomous or moved there from the stand 87, is adaptable preferably to the measurement and evaluation of blood pressure, oxygen in blood content, temperature, pulse, ECG and quantities derived from the aforementioned items, including variability, arrhythmia and all functions and deviations in cardiac activity ascertainable from them, such as PVC, PAC, AV block, pulse drop failure, infarction, arrhythmia, flutter, bradycardia and tachycardia.

These items are evaluated automatically, preferably either in the monitor 349, module 357, 358, where they are also in a displayable format on the display element 1, or they are processed in a more accurate manner in the evaluation block 359 from which they are preferably sent back to the module 357, 358 of the monitored person or the system operator's PC 889 in a data format to be displayed on the display element 1 and/or to be saved in the memory, or they are evaluated by a physician and communicated verbally preferably by a telephone call or e-mail or in any other suitable manner. The described device can be preferably located in a means of transport, such as vehicle or aircraft.

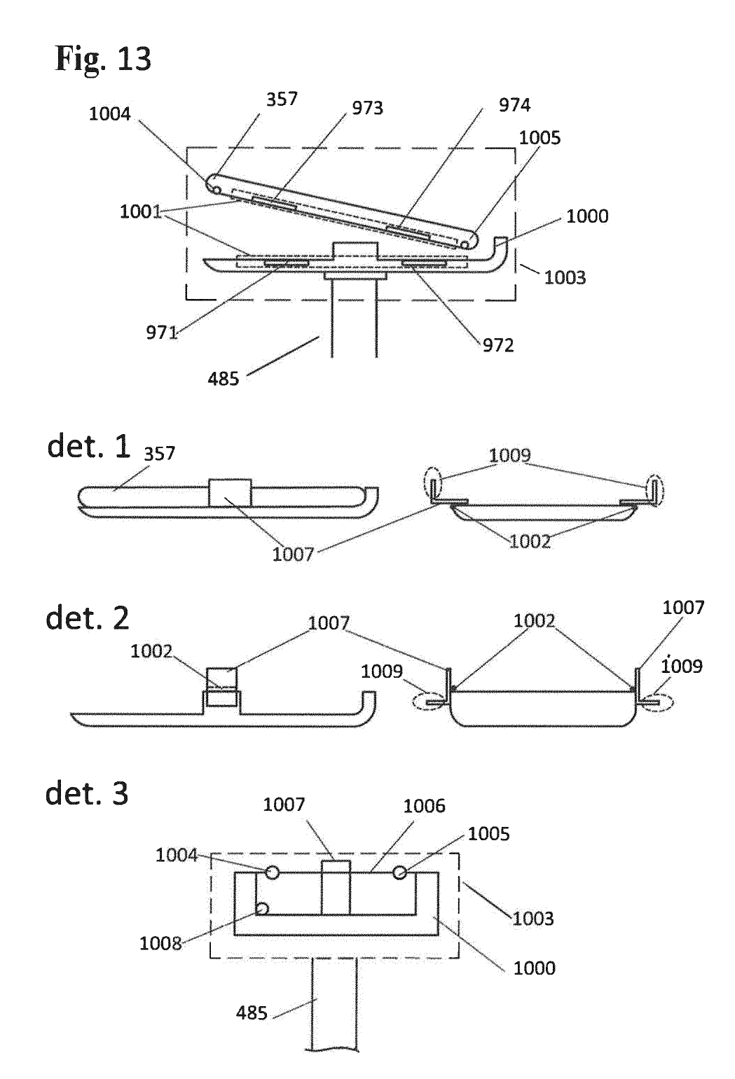

FIG. 13 shows the mechanism 1003 for the detachable mounting of the operative module 357, preferably formed by the magnetic holder 1000 of the controlling and displaying operative module 357 preferably formed by a mobile phone or a mobile watch preferably equipped with a SIM card for telephone communication which is equipped by the system 1001 of magnets, preferably consisting in the magnet 922 which can be preferably duplicated or triplicated to acquire a greater holding force by the countermagnets 974 which are positioned in the body of the module 357. The system 1001 of magnets preferably consists of a minimum of two magnets, the magnet 971 and the second magnet 972 on the body of the holder and with opposing countermagnets being the countermagnet 973 and the second countermagnet 974, on the body of the module 357 in such a configuration that if the module 357 approaches the holder 1000 in an incorrect position, the induced forces automatically draw the module 357 into the correct position in the holder and in this position the monitor then snaps into the holder in such a manner that always the pair of the magnets 971, 973 and 972, 974 are closely attracted to one another always by their opposite poles.

FIG. 13 det. 1 shows the operative module 357 inserted into the holder 1000 secured outside the system 1001 of magnets in addition preferably by the mechanical safety valve 1007 which can be used to secure the position of the module 357 in the holder 1000 under extreme conditions, for example, for sports use.

FIG. 13 det. 2 shows the holder 1000 with the removed operative module 357, with the released safety valves rotatable around the hinges 1002, adjustable in both positions, open and closed, preferably by a dent on the hinge 1002 spindle with the counter dent on the body of the hinge. The safety valve is preferably controlled by the thumb and index finger. For a better grip of the safety valve during opening, it is equipped by the protrusion 1009.

FIG. 13 det. 3 shows the mobile watch 1006 integrated in the smaller mechanism 1003 having the appearance and dimensions and preferably also the function of the watch.

The watch preferably communicates with another mobile phone which the watch preferably controls, receives or initiates telephone calls, displays on it the telephone numbers of the caller or called person, SMS, a list of received and non-received calls, which were performed or not performed from the mobile phone, and/or the watch 1006, along with data from the mobile phone having the function of the module 357. In addition, the watch 1006 preferably receives data from the body of the monitored person via the mobile phone or directly, or it preferably transmits such data to the mobile phone. When receiving a telephone call the watch communicates as a standard telephone, or after removal it is possible to put it to the ear and use the speaker 1004 of the watch as the receiver earpiece and the microphone 1005. The watch 1006 is preferably operated independently without the module 357 as a mobile phone removable using the mechanism 1003 with the wristband 485, and the watch preferably receives health data from the body of the monitored person and preferably controls the monitor 349 and sensors. The watch 1006 is preferably equipped on its bottom side with the headphones 1008 to be put on the ear after the watch 1006 is removed, and on the upper side with the speaker 1004 audible also when put into the mechanism 1003. The watch 1006 is adapted to be operated as the module 357, and for the mobile phone, the parts of the mobile phone are adaptable to be operated as the module 357. In such a case, the watch is equipped with all necessary parts and functions.

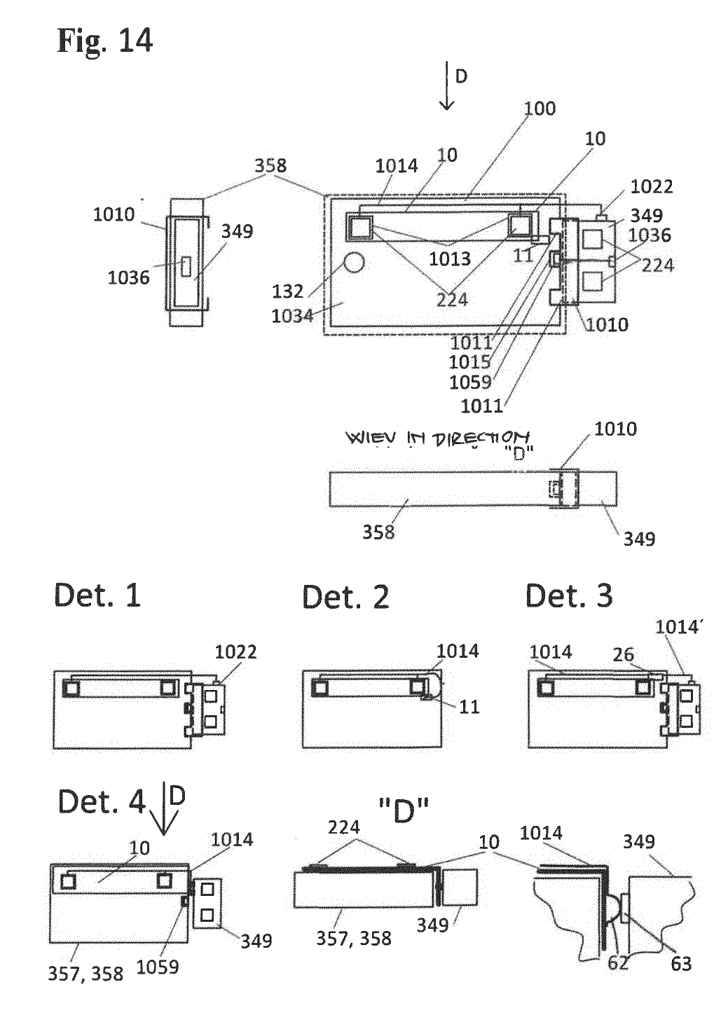

FIG. 14 shows an alternative embodiment of the monitor 349 as a universal one where the monitor 349 is mounted on the controlling and displaying module 358, preferably consisting of any mobile phone 100, with the shown objective 132, cross-connecting connector 1015 of the module, preferably a USB connector, cross-connected to the data connector 1059 of the monitor, whereby the mechanical and electrical connection of both parts is established. The universal character of the monitor 349 has the advantage consisting in the possibility of connecting it to a majority of commonly available mobile phones and the possibility of mounting it on other devices after removal. This connection is preferably supported by the auxiliary mounting 1010 preferably affixed by the protrusions 1011 onto the auxiliary cover 1034 of the module 358 preferably consisting of the mobile phone 100 from which the monitor 349 is detachable. The auxiliary cover is preferably interchangeable with the original mobile phone cover 100 which was removed to allow the auxiliary mounting 1010 affixed to the auxiliary cover to be mounted. Mounting the monitor 349 onto the module 358 without using the auxiliary mounting is shown in det. 1. On the body of the monitor 349 there are preferably two contact areas 224 to be operated as the electrodes 1013 for the fingers of the left and right arms to sense cardiac signals preferably to acquire the ECG or pulse curve. Alternatively, the contact areas 224 for sensing such cardiac signals are used to be attached to the chest. They are positioned on the cover 1034 of the mobile phone 100, preferably on the strip 10 on the cover 1034, preferably by gluing or using a Velcro fastener. These contact areas 224 forming electrodes are connected to the monitor 349 using the electrode short cable 1014 and the electrode connector 1022. These contact areas 224 and preferably the short cable 1014 are preferably attached to the strip 10, preferably by gluing or in a detachable manner preferably using a Velcro fastener, on the spare cover 1034, whereby the protruding short cable 1014 is inserted into the monitor using the connector 1022. In the “D” view it is obvious that the monitor 349 connected in this manner preferably will not increase the thickness of the mobile phone 100. The monitor 349 is powered from the module 358 and communicates with the module using the monitor cross-connecting connector 1059 and the connector 1015 of the module 358 preferably formed by the mobile telephone 100, or communicates with it via a wireless link, preferably Bluetooth-based. The advantage of the direct connection of the monitor 349 to the controlling and displaying module 358 is that the monitor 349 is powered from there and does not need an internal accumulator, and communicates via the connector and does not need a wireless module, such as Bluetooth-based, which allows smaller dimensions to be achieved. The cross-connecting connector 1015 is preferably connected to the second cross-connecting connector 1036 allowing data communication and/or the module 358 power supply even with the connected monitor 349. The switch 118 of connectors preferably switches between the connection of the connector 1015 to the connector 1059 or the connector 1036.

Detail 1 shows the embodiment of the monitor 349 without the auxiliary mounting 1010.

Detail 2 shows how with the disconnected monitor 349 the connector is placed for a temporary period of time preferably into the small pocket 11 on the adhesive strip 10.

Detail 3 shows the short cable 1014, terminated by the strip connector 26 into which is inserted the counterconnector of the short cable 1014′ of the strip, detachable when the monitor is removed.

Detail 4 shows the strip 10 bent around the end of the module 357, 358 with the strip springs 62 pressing against the monitor contacts 63 of the monitor 349 when it is plugged into the connector 1059. Cross-connections using the strip springs 62 and the monitor contacts 63 preferably replace cross-connection using the electrode connector 1022.

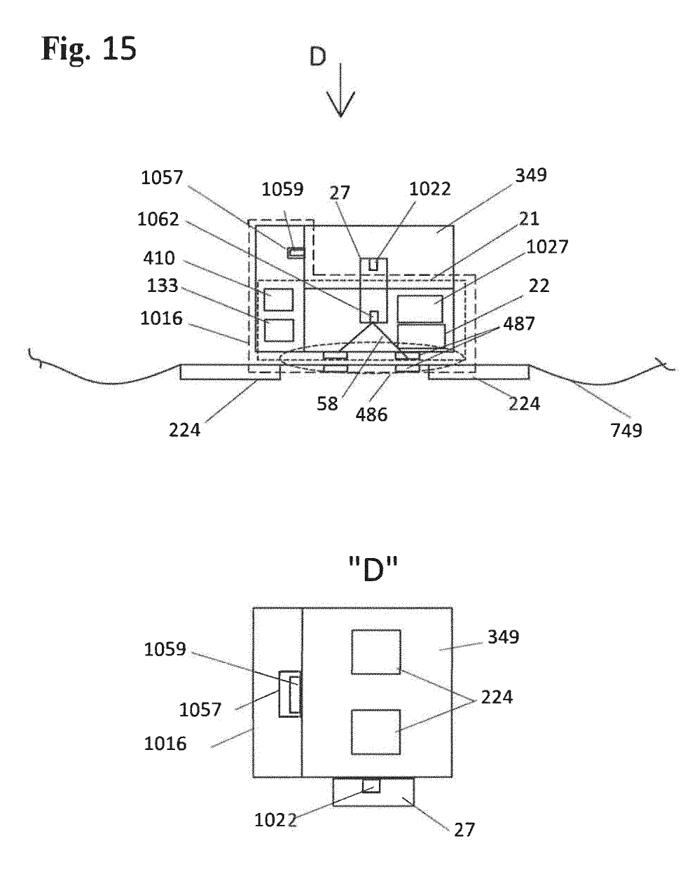

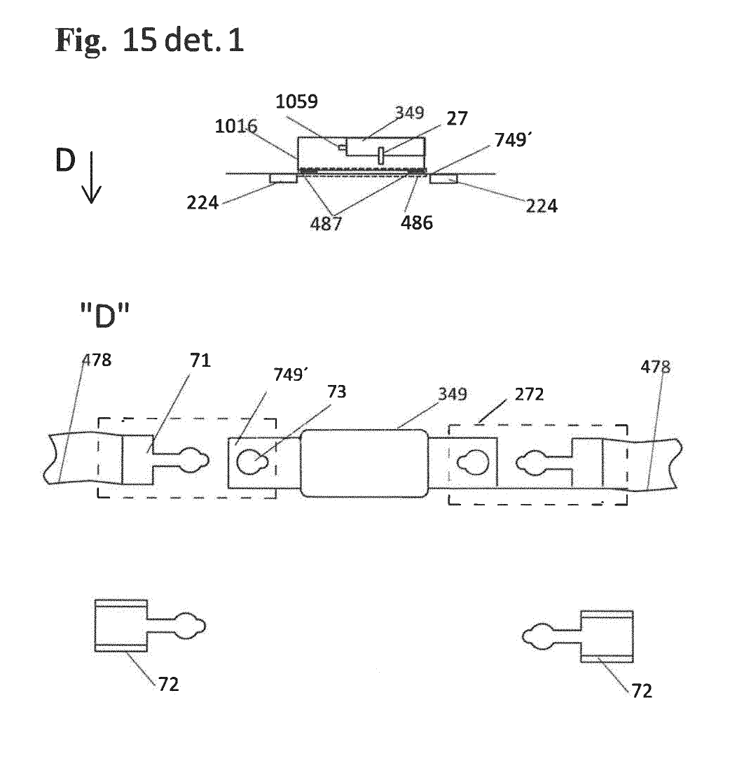

FIG. 15 shows the monitor 349 from FIG. 14 insertable into the base 1016 of the monitor to which it is connected by the monitor data connector 1059 and the electrode bridge 27. The monitor base 1016 is equipped with the additional equipment 21, preferably formed by the accumulator 1027 of the monitor base, preferably replaceable by a recharged one, along with the communication module 22 of the base, preferably formed by the Bluetooth unit 133, module 410 of additional units and other necessary components supplementing the differently equipped monitors 349 which can be used for various applications.

The fastening elements 486, preferably formed by the snap fasteners 487, are used for mechanical and electrical connection to the chest belt 749, or the wristband 485, the snap-fitted plate 488, the shortened chest belt 749′ which can be held down on the chest without using an elastic strap 478 going round, and other equipment onto which the monitor 349 can be replaced. The snap fasteners bringing cardiac signals are connected preferably using the electrode connector 1057, base and bridge 58 to the connector 1022.

The view in the “D” direction shows the top view.

Det. 1 shows the shortened chest belt 749′. It is preferably manufactured from some stiff material preferably flexible one, such as rubber or plastic material, to be put onto the chest where it is held down by hand to make a good contact preferably in the place of the monitor 349 snap-fitted on the snap fasteners 487.

A view in the “D” direction illustrates the fastening of the elastic strap 478 onto the monitor 349 with the base 1016 to be pressed down by hand or attached by the elastic strap 478 going round. In addition, the U-shaped profile 72 to press down the chest belt 749′ using the pressure belt 65 is illustrated.

The monitor 349 thus can be used for sensing cardiac signals both from the contact areas 224 of the chest belt and from the fingers if no chest belt is connected.

The D view provides the top view of the monitor 349.

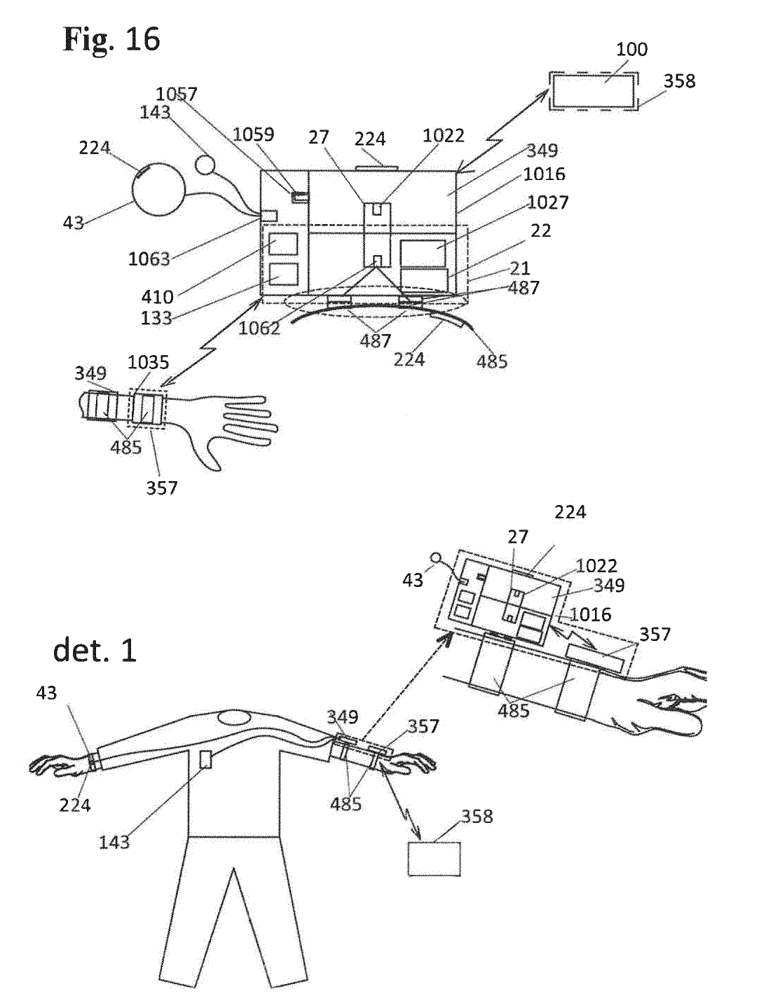

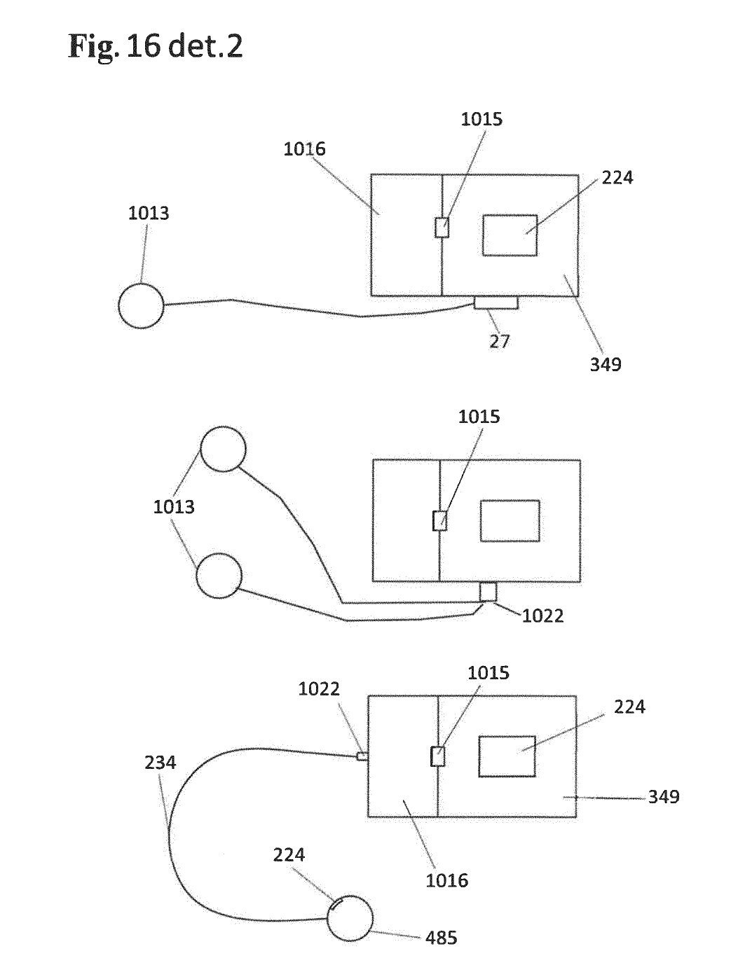

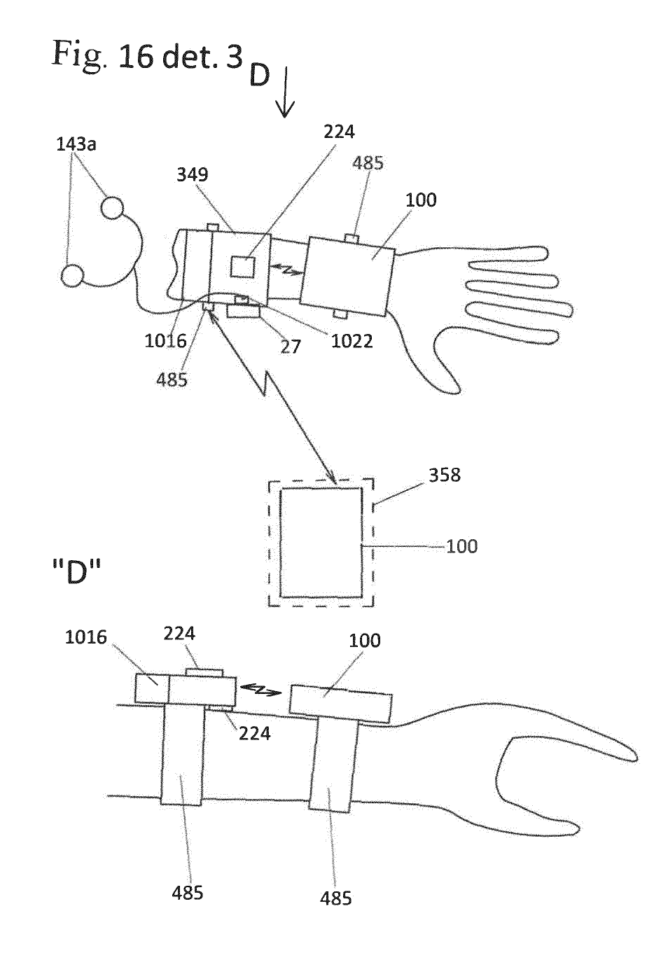

FIG. 16 shows the positioning of the monitor base 1016 on the wristband 485 of the monitored person preferably communicating in a wireless manner with the controlling and displaying module 358, preferably consisting of the mobile phone 100 and/or the communicating controlling and displaying operative module 357 preferably formed by a mobile phone on the wrist. The one arm electrode for sensing cardiac signals is preferably formed by the contact area 224 on the wrist of the one arm positioned preferably on the wristband 485, powered via the snap fasteners 487, or on the monitor base 1016 connected to the monitor 349 via the bridge connector 58, and the other electrode is formed by the contact area 224 positioned on the body of the monitor 349. Alternatively, the other sensing electrode is positioned outside the monitor 349 to which it is connected in a wire manner via the bridge connector 59. It is preferably positioned on the second arm wristband 43 as the contact area 224, or it is preferably attached by gluing as the electrode 143a glued on the chest of the person being monitored in a place suitable for sensing the cardiac signal of the opposite part of the body, which means of the second arm.

Det. 1 shows the monitor 349 connected to the base 1016 of the monitor attached on the wristband 485 with the contact area 224 of the one arm on the upper surface of the monitor 349 and the contact area of the second arm positioned on the wristband 43 on the second arm. The monitor 349 communicates with the controlling and displaying module 358 and/or the controlling and displaying operative module 357 via a wireless link.

The module 357, preferably positioned in a detachable manner on a wristband, is adapted to take over data, telephone calls and SMS coming to the module 358 and display information on such items. Alternatively, the operative module 357 is placed on the wristband in a non-detachable manner and is equipped with a voice unit preferably attachable in a detachable manner on the wristband, which communicates therewith or with the module 358 and makes telephone calls using the microphone and headphones.

FIG. 16. det. 2 shows the variants for the connection of the electrodes to the connector 1022 of the monitor where, for the contact of the one arm, the contact areas 224 on the bottom side of the monitor 349 are used, while the contact of the second arm is realized by the contact areas 224 on the upper side of the monitor 349 or the glued electrode 143 connected via the bridge 27 on the chest or the contact area 224 positioned on the wristband 43 of the second arm. Alternatively, the glued electrodes 143a connected on the chest via the connector 1022 to the monitor 349 are used.

FIG. 16, det. 3 shows a detailed view of the monitor 349 inserted in the base 1016 of the monitor next to it, on the wristband with the contact of the second arm on the bottom area of the monitor.

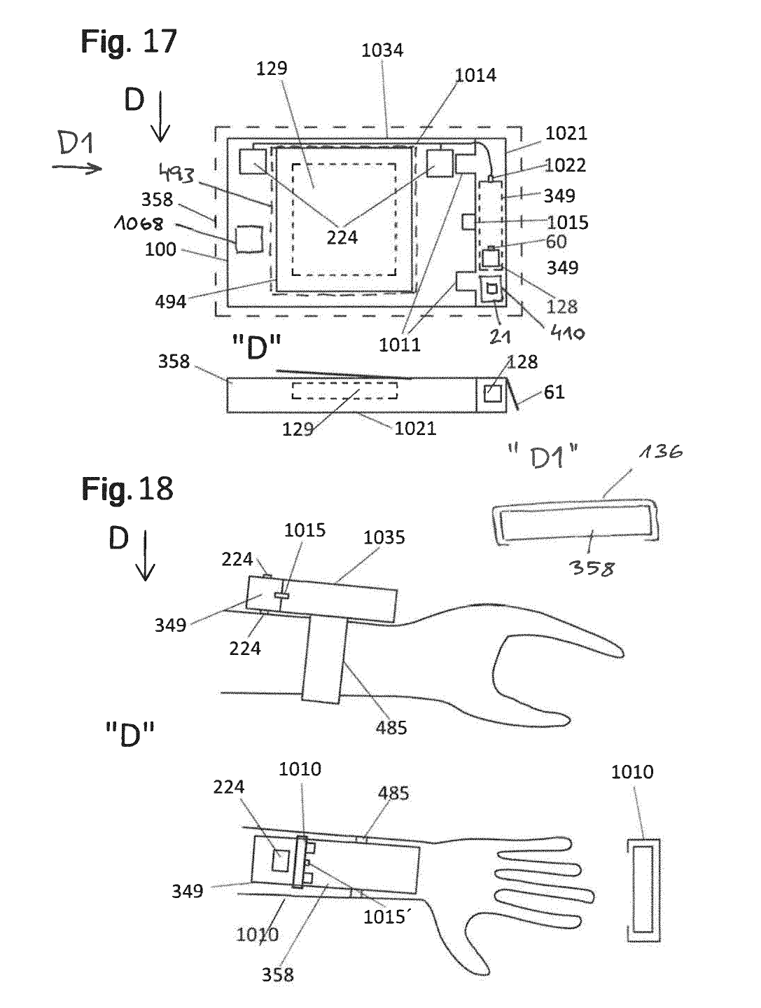

FIG. 17 shows the preferable embodiment where the monitor 349 is placed in a detachable manner in the monitor housing 1021 with the door 61 for the monitor whose shape allows perfect uniting with the controlling and displaying module 358 preferably formed by the mobile telephone 100, giving a visual impression of a single unit. This is preferably implemented by gluing the cover of the module 358 to the protrusions 1011 of the housing 1021 or by creating of an extended cover replacing the original cover in a manner giving a visual impression of a single unit. The electrodes 1013 for sensing of cardiac signals are placed on the cover of the controlling and displaying module 358 preferably formed by the mobile phone 100, preferably connected to the connector 1022 of the electrodes using the short cable 1014. In the case that the module 358 is not equipped with a removable cover, where such a cover is formed by the supplementary cover 136, visible in the D1 view, which can be used in all the remaining illustrated examples where the removable cover is illustrated, and is preferably attached by going round the module 358 from the sides which allows taking away.

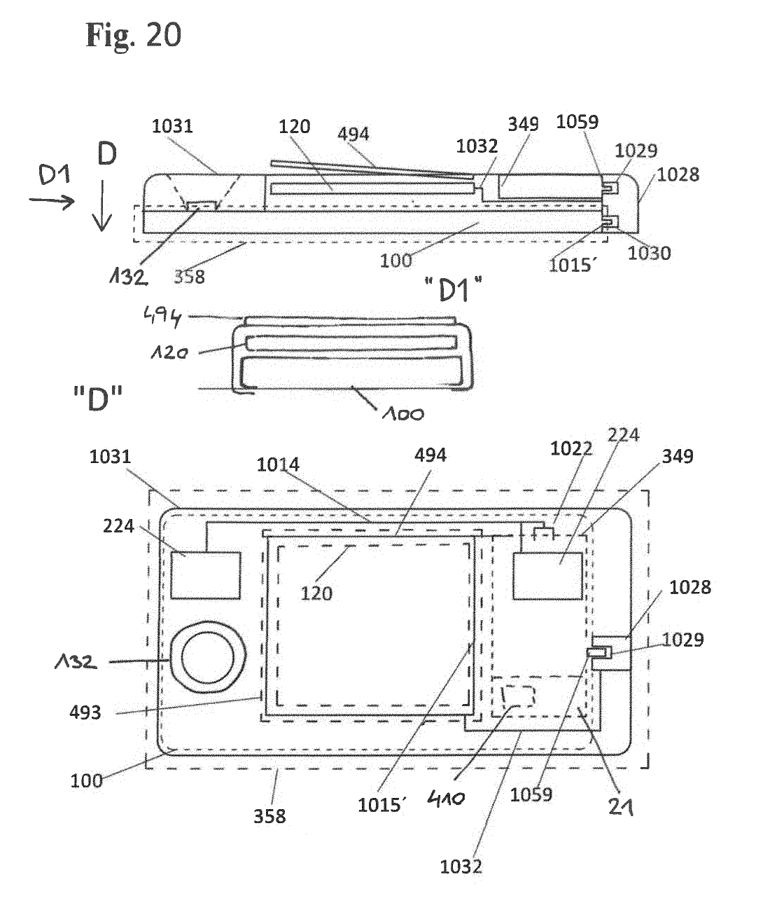

The monitor housing 1021 preferably includes the bridging accumulator 128 connected by the connector 60 of the bridging accumulator which allows, via the monitor 349, to power the controlling and displaying module 358 preferably comprising the mobile phone 100 at the time of replacement of its main accumulator 129 by the recharged one, which thus is advantageously realized without interrupting the module 358 operation. If the module 358 is not equipped with the replaceable accumulator 129, the solution based on the additional accumulator 120 provided in FIG. 20 is preferably used. In addition, the cover 136 preferably includes, preferably in a detachable manner, the block 21 of additional equipment allowing data communication with the module 358 comprising optional additional units where necessary, preferably independently operated microprocessor unit 1068 of the module 358 for example with a different operating system than that of the microprocessor in the module 358. This arrangement will allow two operating systems to be used, for example Android and Symbian. The block 21 preferably includes the module 410 of additional units which can be replaced together with the connected blocks 21 in the housing 136 by ones equipped with various accessories as necessary.

FIG. 18 shows the monitor 349 inserted into the cross-connecting connector 1015 of the controlling module 358 preferably comprised by a mobile phone on wristband. The electrode of the one arm for the wrist, preferably implemented by the contact area 224, is preferably placed on the bottom part of the monitor 349, while the electrode, the contact area 224 for the fingers of the second arm, is positioned on its upper part. The view in the D direction shows the housing 1021 of the monitor for the telescopic attachment of the monitor 349 attached by the protrusions 1011 to the cover of the controlling module, or the housing 1021 is implemented as an extended cover replacing the original cover of the module 357.

The monitor 349 is powered and communicates via the cross-connecting connector 1015 with the controlling and displaying module 358, or communicates therewith using a wireless link preferably formed by a Bluetooth module.

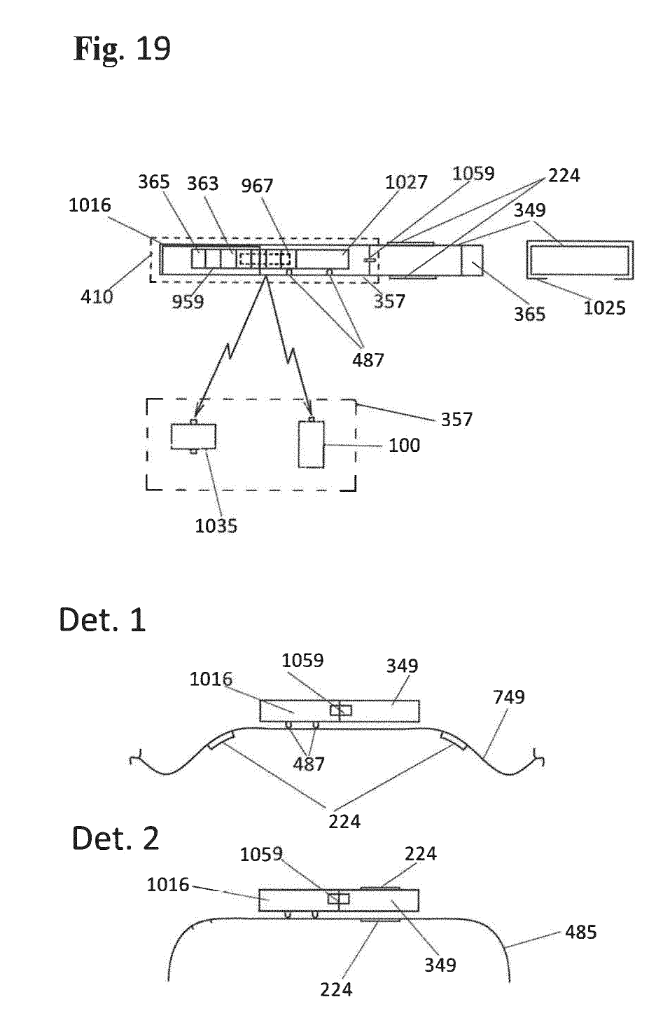

FIG. 19 shows the monitor 349 connected to the base 1016 as an extension using the data connector 1959 of the monitor and the slide-on holder 1025 as an alternative to the assembly provided in FIG. 15. In such an arrangement the bottom electrode 1023 for fingers is led out onto the bottom part of the monitor base 1016. Both embodiments can be fastened using snap fasteners, as illustrated in FIG. 20 det. 1 and 2, on the wristband or chest belt 749. The detached monitor 349 is connected by the cross-connecting data connector 1059 of the monitor to the base 1016 preferably equipped by the monitor base accumulator 1027 and/or the communication module preferably formed by a Bluetooth unit communicating with the controlling and displaying module 358 preferably formed by the mobile phone 100, and/or the controlling and displaying operative module 357 preferably formed by the mobile phone 100 preferably positioned on the wristband. In addition, the monitor base 1016 is preferably equipped with other units, if necessary, preferably with the SD card 959, parts for communication via a mobile operator's network, a SIM card, an optical front-end for pulse sensing, preferably with a fall detector, receiver and or transmitter for a wireless link in the 5 kHz band, and other units in the module 410 of additional units. The monitor is preferably equipped with its own control unit. Various monitor bases 1016 are preferably mounted to the monitor where necessary with various accessories. The fastening elements 35 are preferably comprised of the snap fasteners 487 allowing the housing of the monitor 1016 to be attached to the monitor 349 onto the chest belt 749 or the wristband 485.

The slide-on holder 1025 has a shape allowing the monitor 349 to be inserted into it.

The monitor housing 1021 preferably extends the base and copies the shape of the monitor 349.

FIG. 19 det. 1 shows the attachment of the monitor 349 to the chest belt 749.

FIG. 19 det. 2 shows the attachment of the monitor 349 to the wristband 485.

FIG. 20 shows the monitor 349 connected preferably using the data connector 1059 to the bridge 1028 and its first bridge connector 1029 where the second bridge connector 1030 is connected by the connector 1015′ to the controlling and displaying module 358 preferably formed by the mobile phone 100. The monitor is preferably placed in the additional cover 1031 preferably snap-fitted on the module 358. The monitor 349 is mounted, preferably when the additional cover 1031 is in detached state, where there is also the additional accumulator 120, that, via the bridge 1028 to which it is connected by the short cable 1032, supplies power to and/or recharges the module 358 and preferably the monitor 349

The accumulator 120 is placed in a detachable manner in the additional cover 1031 using the fastening mechanism 493 by which it is possible to replace it preferably without interrupting of operation of module 358. The fastening mechanism 493 preferably comprises the door 134 for an easy removal of the accumulator 120.

The monitor 349 can be removed from the additional cover 1031 and after removal it can be used for the connection of the monitor base 1016 to the accumulator 1027 as illustrated in FIG. 14, which replaces power supply from the accumulator 120 or from the module 358. To save space, the monitor 349 is left preferably without the accumulator and wireless module which are preferably included in the base. The additional cover 1031 is mounted onto the module 358, preferably instead of its cover, or snap-fitted over the module 358. On the additional cover 1031 the contact areas 224 are preferably positioned to be applied to the fingers or chest; they are connected by the electrode short cable 1014 to the electrode connector 1022. The additional cover 1031 is equipped with an opening for the objective 132. The additional cover 1031 preferably includes, preferably in a detachable manner, the block 21 of additional equipment being preferably replaceable by a different one equipped as necessary. In this manner, the blocks 21 equipped with various accessories can be installed to supplement the module 358 by optional functions. The block 21 preferably includes the module 410.

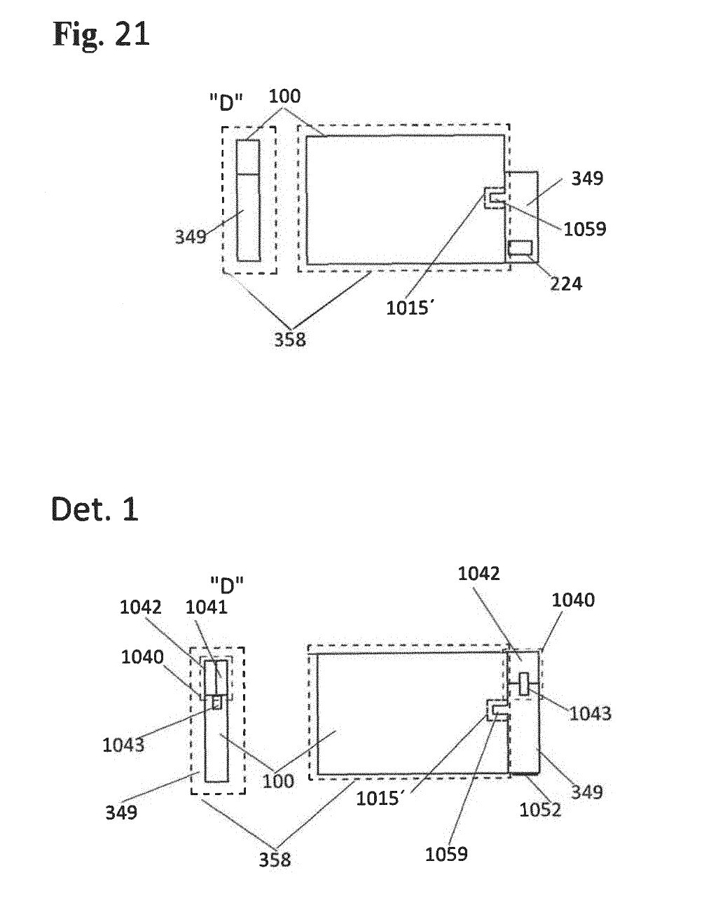

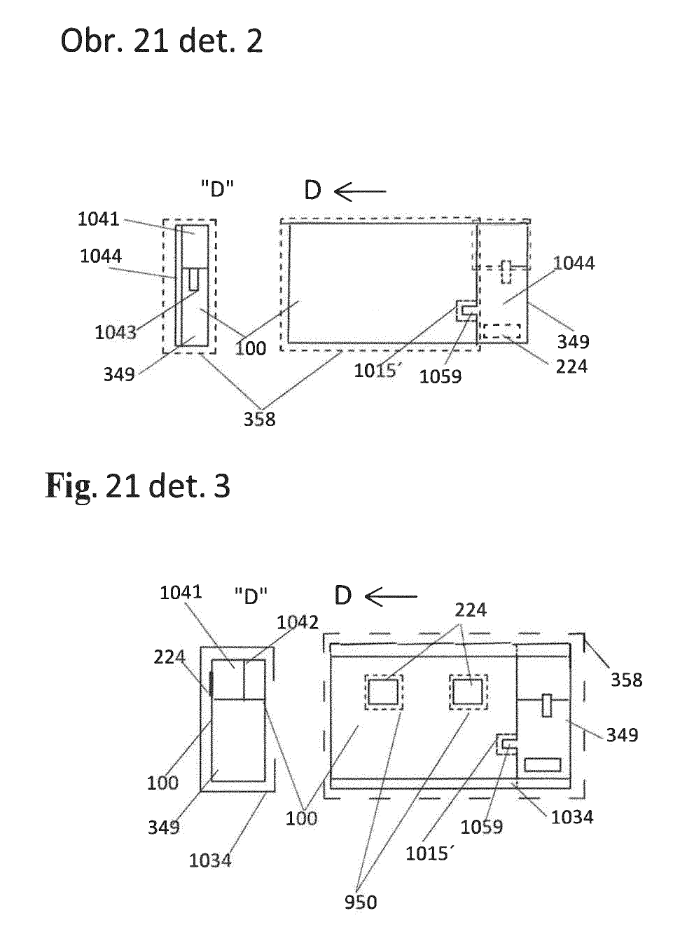

FIG. 21 shows the embodiment of the monitor 349, where it is connected to the controlling module 357, 358, preferably formed by the mobile phone 100, by inserting into the cross-connecting connector 1015 of the module via the data connector 1059 of the monitor preferably positioned eccentrically to create space for placement of the display control unit 1041. The contact areas 224 are preferably located on the sides and at the bottom if viewed in direction to display

In FIG. 21 det. 1 the monitor 349 is equipped with the displaying unit 1040 consisting of the display control unit 1041 and the small display 1042. The displaying unit is connectable to the monitor 349 using the side connector 1043 so that it creates a compact unit with the monitor 349 and the module 357, 358. The monitor is equipped with the push-button 1052 for the test start.

FIG. 21 det. 2 shows the embodiment of the monitor 349 inserted into the cross-connecting connector 1015, where its displaying unit 1040 is equipped with the large display 1044, whose size covers nearly the whole front side of the monitor 349 preferably forming with the monitor control unit 365 a whole being visible if the display of the module 357, 358 is viewed.

FIG. 21 det. 3 shows the type embodiment of the monitor 349 which is designed for the specific module 357, 358, where its cover 1034 is modified to allow connection and common housing of the monitor 349 and the displaying unit 1040. This solution is similar to that provided in FIG. 14, but the monitor is adapted to the use of the small display 1042 or the large display 1044 illustrated in FIG. 21 det 1 and 2. The cover is equipped with the contact areas 224 designed for sensing the signal of the one arm and the signal of the second arm, either by pressing down fingers or applying the cover onto the chest of the monitored person.

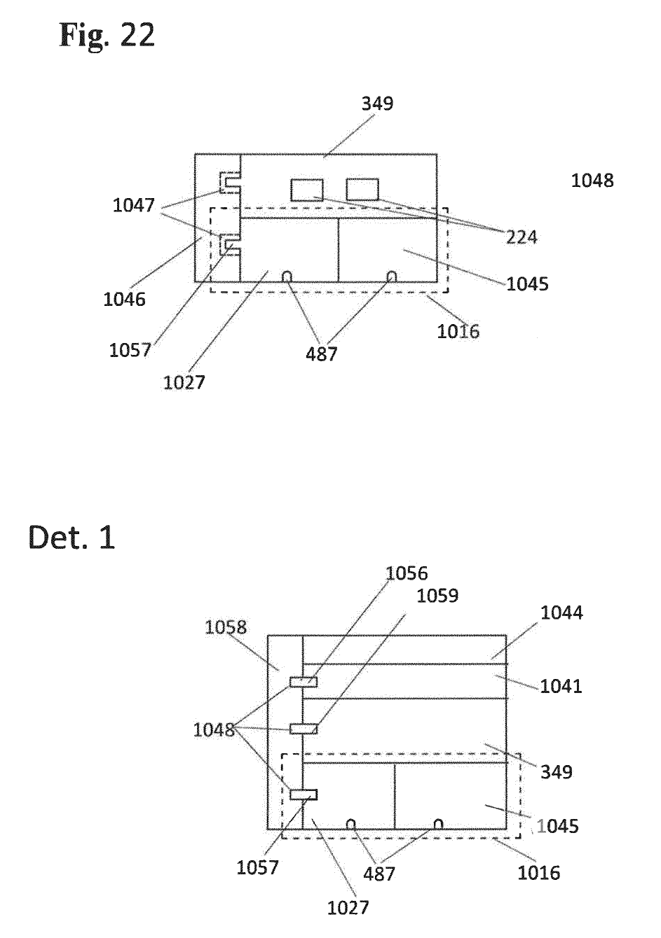

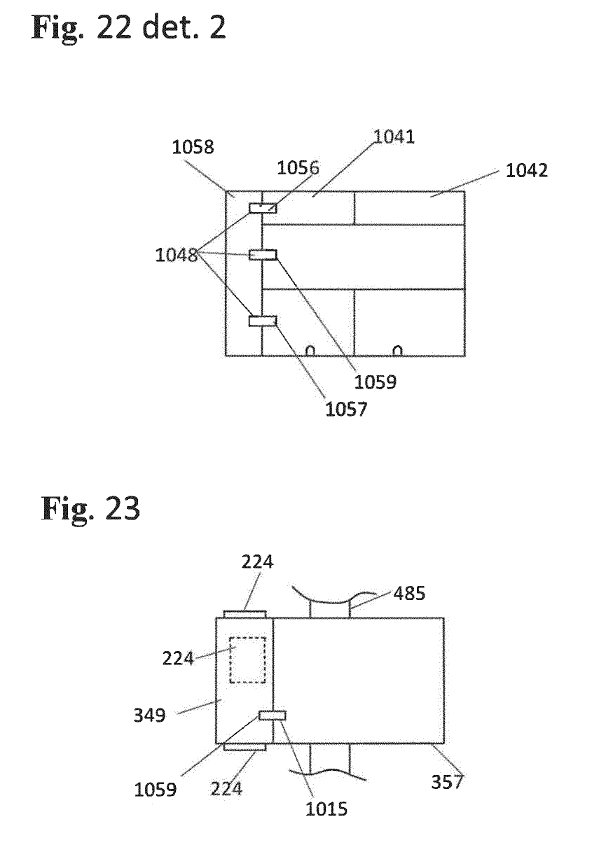

FIG. 22 shows the variant of the monitor 349 for short-term tests where the monitored person deliberately touches the contact areas 224 for a short period of time. The monitor 349 is placed into the monitor base 1016 comprising the control unit 1045 of the base, the accumulator 1027 of the monitor base, the double cross-connecting bridge 1046 and the snap fasteners 487 to connect and attach the monitor according to the preceding embodiments. The bridge is equipped with the connector link 1047 into which the monitor 349 is connected via the connector 1059 base 1016 via base connector 1057. The monitor is equipped with the contact areas 224 for sensing the signal of the first arm and the signal of the second arm.

The bases 1016 with various accessories are used where necessary.

FIG. 22, det. 1 shows the extended embodiment from FIG. 22, where the bridge is comprised of the triple-bridge 1058 that via the connector link 1048 also ensures the connection of the display control unit 1041 and the large display 1044 via the display connector 1056.

FIG. 22, det. 2 shows a similar embodiment as that provided in FIG. 22, det. 1 with the difference that the small display 1042 located next to the display control unit 1041 is used, which results in a lower height of the entire assembly.

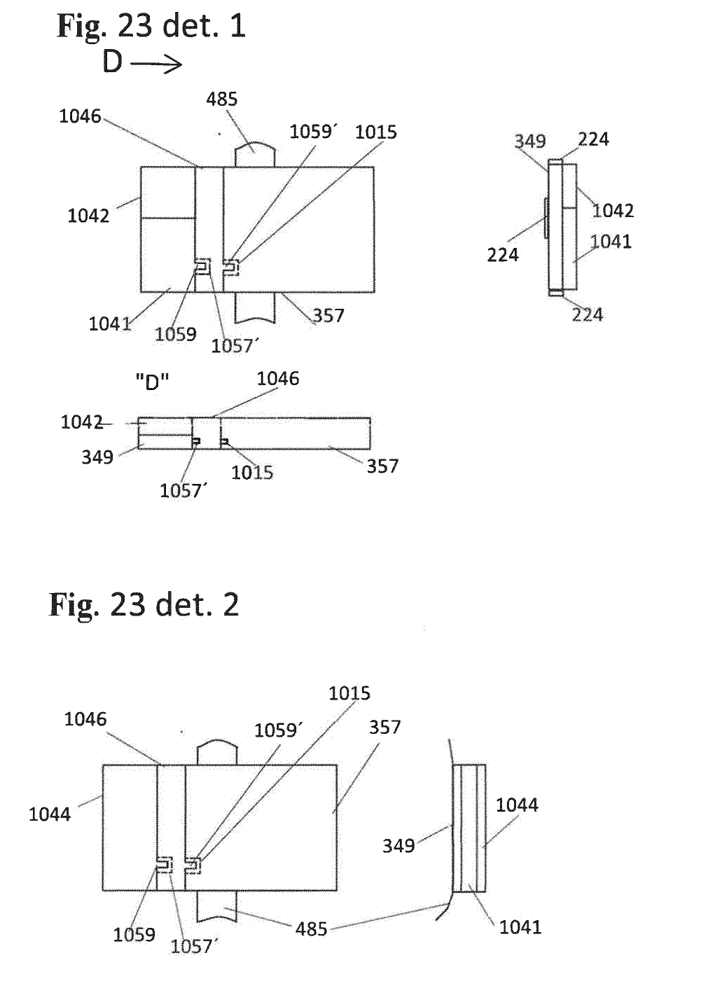

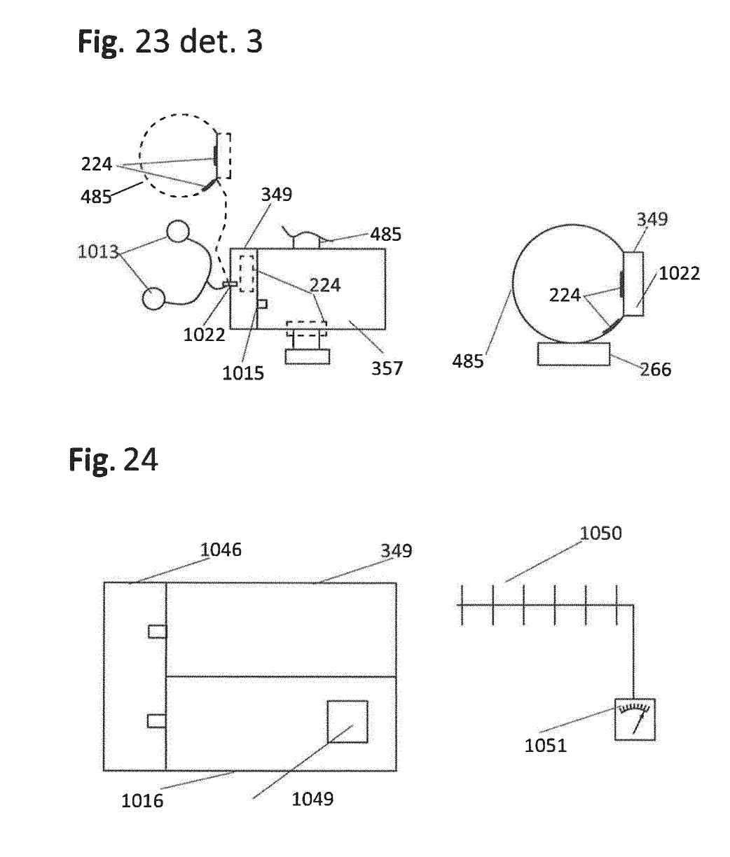

FIG. 23 shows the solution of the monitor 349 connected via the monitor data connector 1059 to the controlling and displaying operative module 357 positioned in a removable manner on the wristband 485. The monitor is equipped with the sensing contact areas 224 arranged in a manner allowing the signal of the first arm to be sensed by the contact area on the bottom side of the monitor 349 and the signal of the second arm to be sensed by pressing the fingers of the second arm against the other contact area 224 located on the side of the monitor body.

FIG. 23 det. 1 shows the embodiment of the monitor provided in FIG. 23 equipped in addition with the display control unit 1041 and the small display 1042, to which it is connected by the connector 1059 in the double cross-connecting bridge being an intermediate element between the monitor 349 and the operative module 357. The connector 1057′ of the first bridge provides connection with the module 357 via the second bridge connectors 1059′ and the module connector 1015.

FIG. 23 det. 2 shows the embodiment of the monitor 349 provided in FIG. 23, det 1 where, however, the large display 1044 above the control unit 1041 is used.

FIG. 23 det. 3 shows the embodiment of the monitor 349 cooperating with the operative controlling module 357 however designed also for long-term tests. In this embodiment the monitor 349 is equipped with the electrode connector 1022 designed for a wire link of the electrodes sending the signal of the first arm, or where applicable that of the second arm, using the electrodes 1013 or the contact areas 224. These electrodes are positioned either on wristbands on the wrists and/or are glued on suitable parts of the body as described in FIG. 16 det. 3. Alternatively, the electrode of the first arm is arranged based on the contact area 224 on the bottom side of the wristband 485. In the case of wire-connected electrodes the operative module is fixed in its position on the wristband 485, and therefore the communication of the person under monitoring using the operative module 357 is enabled by the removable voice unit 266 which is positioned in a removable manner on the wristband 485 and connected to the controlling operative module in a wireless manner, preferably by a Bluetooth unit, and allows “hand-free” communication.

FIG. 24 shows the variant of the monitor 349 with the localizable transmitter 1049, namely in the embodiment with the monitor base 1016. This principle can be applied to any embodiment of the monitor 349. The transmitter transmits at a constant energy level, preferably at time intervals allowing to be energy efficient. Its position is, depending on the signal power, localizable by the directional antenna 1050 connected to the measuring receiver which, depending on the signal intensity, preferably determines the approximate distance from the transmitter. Cooperation of two such receivers can localize the position of the transmitter as an intersection of two ascertained directions. Such a localization of the transmitter's position is used where no GPS system is available, where the module 358 transmits information on its current position, preferably via a mobile operator's or a Wi-Fi network.

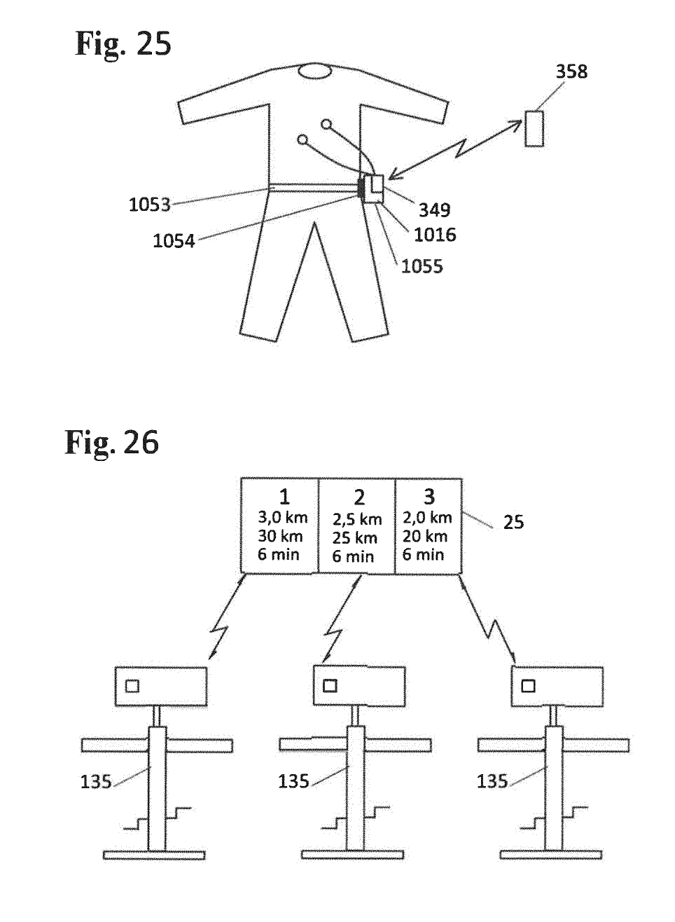

FIG. 25 shows the monitor 349, positioned on the base 1016, which is mounted on the belt 1053 on the body of the person being monitored, using the housing 1055 or the spring clamp 1054 preferably mounted to the base 1016 using the snap fasteners 487. For sensing cardiac signals some of the aforementioned contact areas 224 are used, preferably two glued electrodes 143a connected to the monitor 349 using a wire link implemented by the electrode connector 1022. The number of electrodes is extensible to a larger number to bring signals for up to twelve ECG leads.

FIG. 26 shows three Rotopeds 135 equipped with the monitors 349, which transmit, preferably in a wireless trimmer, (continuous) results of cycling, preferably a minimum of “mileage”, speed and time, on the shared screen 25. Upon reaching the finish adjustable by the number of kilometers according to the number of cycles of pedals with adjustable load, time for individual Rotopeds and thus the standing of contestants is measured. In the case of a handicap, various loads are adjusted based on the contestants' performance. In addition, medical information and information on cardiac activity is preferably displayed on the module 358.

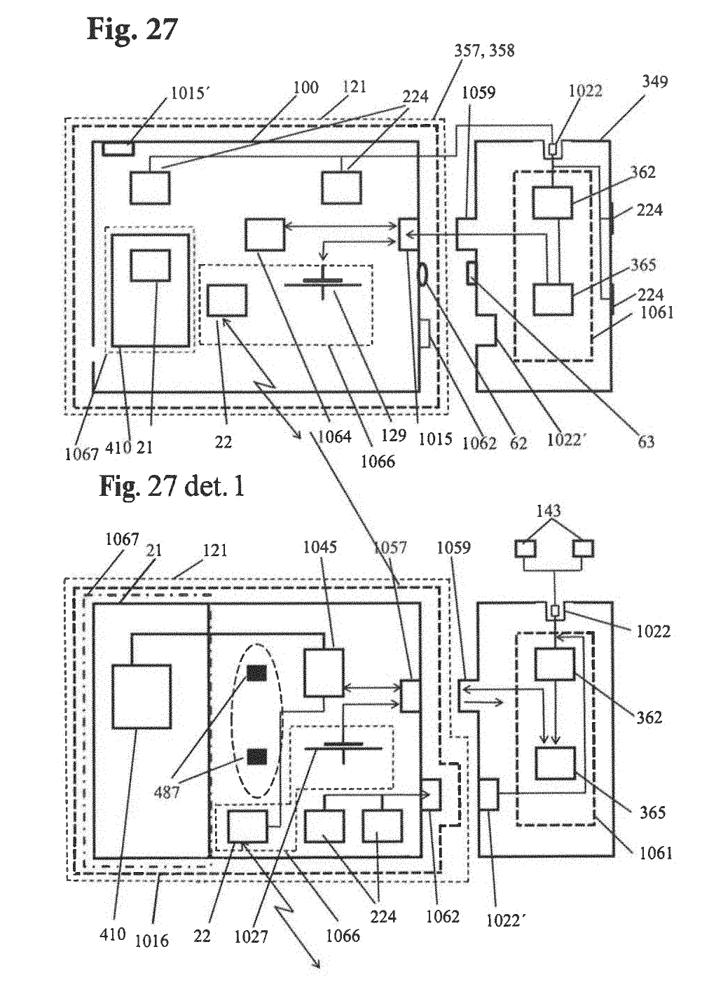

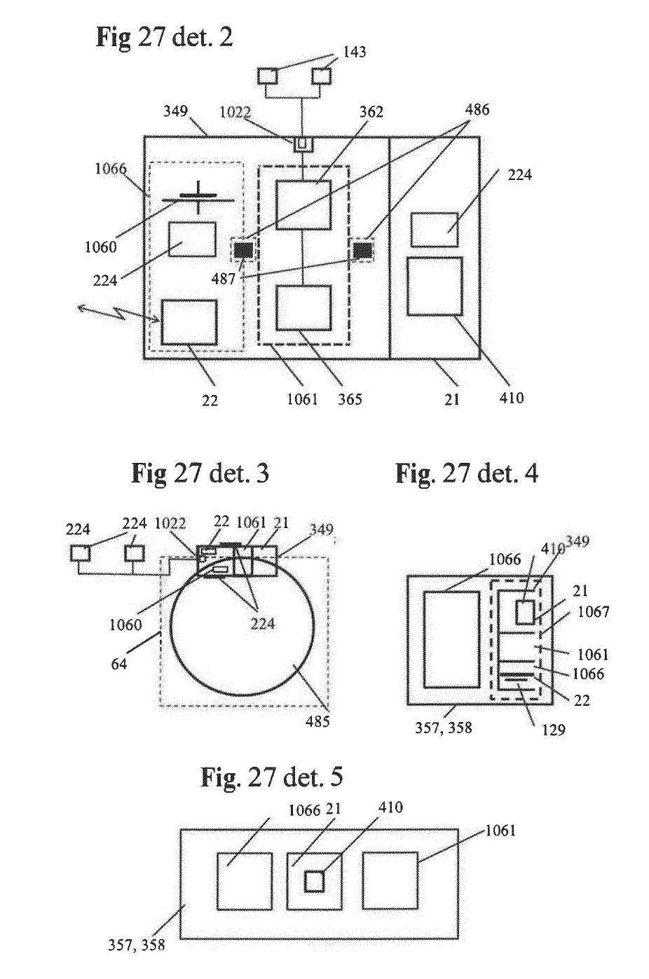

FIG. 27 shows the types of the monitor 349 in a clear manner. The basic type of the monitor 349 illustrated also in Detail 1, comprises only the block 1061 of basic units, preferably comprising the front end 362 and the control unit 365. The block 1066 of supplementary units, which preferably ensures power supply by the accumulator 129 and communication via the communication block 22, is positioned in the cooperating unit 121 where all optional functions are ensured by the block 21 of supplementary units comprising additional units and equipment, and the module 410. The cooperating unit 121 to which the monitor 349 is connected is preferably formed by in particular the controlling and displaying module 358, the controlling and displaying operative module 357 or the monitor base 1016. The fact that the monitor 349 includes only the block 1061 results in the following benefits: the small size and low price of the monitor and the possibility to opt for cooperating units with various accessories 121. The monitor 349 is connected in terms of data and power supply to the cooperating unit 121 using the connector 1059, preferably USB, for power supply and data. The contact areas 224 or the electrodes are preferably connected using the electrode connector 1022. Alternatively, the electrodes are connected using the strip springs 62 and the monitor contacts 63 or the connector 1062 and 1022′, or via the connector 1015 and 1059 if no USB is used. Preferably it is possible to use another second connector 1015′, preferably a USB one providing data and power supply for the module 357, 358 preferably comprised of the mobile phone 100, as a common USB connector which is preferably positioned outside the contact area of the connected monitor so that its use is possible with the connected monitor 349 as well. The block 21 of additional equipment is both in the module 358 and in the operative module 357 and in the monitor base 1016 replaceable by other ones with various accessories as necessary. The replacement is carried out by the fastening mechanism 1067 of the block. The transportable self-contained type of the monitor 349 illustrated in Detail 2, is designed for independent operation, including power supply. In addition to the basic units 1061 it contains the additional equipment 21, preferably including the module 410 of additional units. The self-contained type is mountable, using the mounting elements 486 preferably formed by the snap fasteners 487, to the auxiliary devices 64, preferably formed by the chest belt 749, 749′, or the wrist band 485 or other auxiliary devices preferably allowing not only mounting but also sensing cardiac signals using the contact areas 224 positioned on them.

By inserting the basic type of the monitor 349 into the monitor base 1016, which is equipped with the mounting elements 486 preferably formed by the snap fasteners 487 allowing the basic monitor 349 to be mounted on the auxiliary devices 64, the basic type of the monitor 349 fulfills functions attributable to the self-contained type.

The self-contained type is preferably manufactured with various accessories of the attachment devices 21, or the block 21 of additional equipment is replaceable, preferably using a connector, and the advantage is that thanks to the fastening elements 486, preferably formed by the snap fasteners 487, the self-contained type of the monitor 349 is substitutable on the auxiliary devices 64 depending on current needs.

Det. 3 shows the fixed type of the monitor 349, which is firmly attached to the auxiliary device 64, preferably the wristband 485. The advantage is that the same monitors 349 are mountable to various auxiliary devices, whereby the number of devices whose production is identical is increased and the production costs decreased.

Det. 4 shows the type of the monitor 349, in the given example integrated into the module 357, 358, which is beneficial due to the fact that the same monitors 349 can be mounted into various devices resulting in increased production volumes and decreased production costs. The monitor 349 is preferably removable and replaceable by the monitor 349 with different accessories using the fastening mechanism 1067, including the block 1061 of basic units, the block 1066 of supplementary units, the block 21 of additional equipment, or only the block 21 of additional equipment is replaceable independently. The block 1066 of supplementary units is preferably integrated in the module 357, 358.

In the detailed description of the block connection the FIG. 27 illustrates the preferred solution of the monitor 349, where the monitor is connected to the cooperating unit 121, preferably to the controlling module 357 or the module 358, preferably formed by the mobile phone 100, via the connector 1959 and the cross-connecting connector 1015 of the module, being also connected to the contact areas 224 on the body of the mobile phone sensing cardiac signals, via a short cable and the electrode connector 1022 that transfers these cardiac signals to the monitor basic unit 1061, namely to the front-end 362 and further to the monitor control unit 365 that processes such signals. It communicates with the mobile phone 100 via the aforementioned connector 1059.

The module 358, preferably formed by the mobile phone 100, processes, displays and preferably sends the results using its wireless interfaces to the server 806 of the surveillance centre 762 and to the remote participants 88.

The monitor 349 is powered from the main accumulator 129 of the mobile phone 100.

FIG. 27 det. 1 shows the solution where the monitor 349 described in FIG. 27 is inserted into the monitor base 1016. In this embodiment cardiac signals are transferred alternatively either from the contact areas 224 positioned on the monitor base and connected to the monitor 349 by the connector 1022′, and/or from the electrodes connected via the connector to 1022. The monitor 349 is further connected to the base 1016 using the data connector 1059, via which it is also powered from the accumulator 1027 of the base. The base 1016 includes the block 21 of additional equipment preferably including the module 410 of additional units.

In FIG. 27 det. 2 shows the self-contained embodiment of the monitor 349 designed for fastening using the fastening mechanism, preferably the snap fasteners 487, to the chest belt or another suitable place, if for cardiac signals sensing external electrodes brought to the monitor via the connector 1022 or the contact areas 224 positioned on the monitor are used. The monitor includes the block 1061 of basic units with the front end 362 and the monitor control unit 365 along with the block 1066 of supplementary units with the communication module 22 and the accumulator 1060. In addition, it contains optionally the block 21 of additional equipment with the module 410 of additional units which is preferably replaceable by a different block with different accessories.

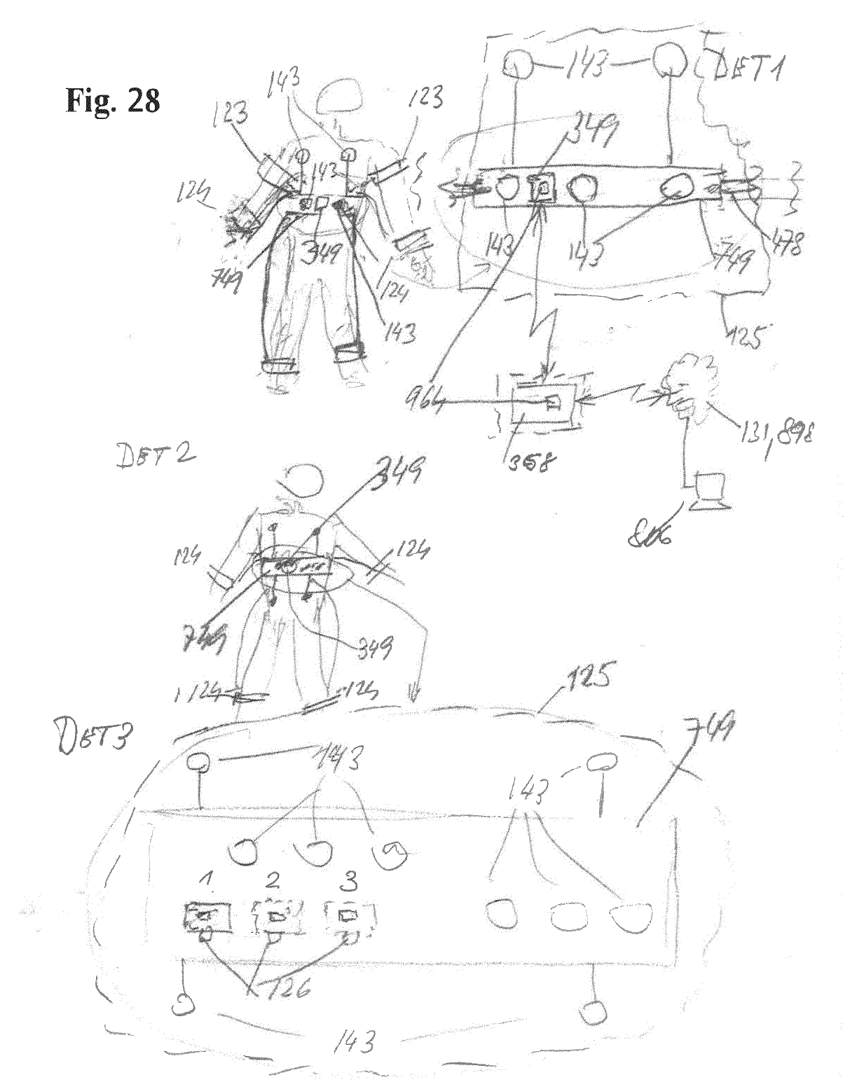

FIG. 28 shows the monitor 349 transferable onto the chest belt 749, fastened using the mounting elements 486, where part of the electrodes 143 is preferably positioned on the chest belt 749 and part of the electrodes 143 is positioned on the body of the monitored person either by gluing, or using the fastening tapes 123 or the clips 124 on the arms, and cross-connected using wires. The advantage is the positioning of part of the electrodes 143 on the chest belt 749 and the connection of other electrodes by wires, resulting in the electrode system 125 being easily installed on the body of the monitored person compared to individual electrodes.

Detail 1 schematically shows the electrode system 125 in magnified form. This system is preferably usable for a Holter with record on the extractable memory medium 964 or it is transmitted, preferably in a wireless manner, to the module 358 or the operative module 357, from which it is preferably transmitted to the server 806 via the wi-fi network 131 or the mobile operator's network 898.

Detail 2 shows the monitor 349 replaced to the chest belt 749 for a 12-lead ECG, in magnified form in det. 3.

The 6 electrodes 144 are positioned preferably on the wide chest belt 749, which allows the optimal arrangement of the electrodes for sensing a 12-lead ECG, namely preferably three and three electrodes in rows with a difference in height. The 4 electrodes are connected by wires, namely the electrodes on the clips 124 on the arms and legs, or they are glued or sucked to the body in places producing the same or a similar signal as the electrodes 143 on the arms and legs allowing a 12-lead ECG to be sensed.

The monitor 349 is preferably positioned in a detachable manner on the belt 749 in one point, and is adapted for sensing a 12-lead ECG, or a monitor is adapted for sensing for part of the leads only. In the case of 6 leads, the monitor 349 is to be moved after sensing of 6 leads in position 1 to position 2 for sensing 6 more leads, a total of 12 leads. When adapted to 4 leads, it is moved three times to positions 1, 2 and 3 for sensing 12 leads. With each move the monitor 349 is connected via the mounting elements 486 preferably formed by the connectors 126 for the respective electrodes.

The advantage of the monitor moving to the chest belt connectors 126 is the saving realized in respect of the monitor designed for only 4 or 6 leads.

The advantage of the electrode system 124 with the 6 or 4 electrodes 143 connected by wires rests in easy installation for sensing of an ECG and following dismantling as a whole compared to individual electrodes connected by wires.

The 12-lead system is preferably used for short-term sensing, such as the acquisition of an ECG on the PC 889 where the monitor is preferably connected wirelessly, or for long-term sensing stored preferably on the extractable memory medium 964 or transmitted to the server 806.

The system 125 is useful for the monitoring of phenomena which cannot be detected by a fewer-lead ECG, for example infarction or the points with the incidence of extrasystoles or bigeminies. With long-term sensing it is advantageous to display on the display, preferably of the module 358, 357, the information about occurrence of ventricular extrasystoles and quantity per a unit of time for the assessment of cardiac activity preferably in athletes and monitored persons with a heart disease.

### INDUSTRIAL APPLICABILITY

The universal chest belt can be used both for sports and for the prevention of arrhythmia and also for medical purposes.

1. An equipment for detecting of the state of health of the monitored persons, characterized in that
it comprises a monitor (349) of heart signals, which monitor is adapted for sensing and processing of heart signals of the monitored persons, and this both for the short-time and the long-time sensing and processing,
wherein the short-time sensing is carried out by means of touching the sensing elements (1065) with fingers and/or with a palm, which touching is carried out by the monitored person or by means of applying said sensing elements (1065) to the body of the monitored person and said long-time sensing is carried out by means of said firmly fixing sensing elements (1065) on the body of the monitored person,
wherein a monitor (359) is transferrable between cooperating units (121)
and/or by means of pieces of auxiliary equipment (64), which pieces are formed preferably by at least one controlling and displaying operative module (357), a controlling and displaying module (358), a monitor base (1016), a wristband (485), a chest belt (749), a control element (31).

2. The equipment for detecting of the state of health of the monitored persons according to claim 1, characterized in that
said main monitor (349) of heart signals comprises a block (1061) of basic units of said monitor, and via a data connector (1059) of said monitor it is connectable to said cooperating units (121), wherein it is transferrable between said cooperating units (121).

3. The equipment for detecting of the state of health of the monitored persons according to claim 1, characterized in that
said self-supporting monitor (349) of heart signals comprises said block (1061) of basic units of said monitor, a block (1066) of supplementary units, and a block (21) of pieces of additional equipment, and it is connectable to said pieces of auxiliary equipment (64) by means of mounting elements (486) and it is communicating wirelessly by means of a communication module (22).

4. The equipment for detecting of the state of health of the monitored persons according to claim 1, characterized in that
said main monitor (349) of heart signals is adapted to fulfill the features of said self-sufficient monitor by being connected to said base (1016) of said monitor, which base comprises a block (1066) of supplementary units, a block (21) of pieces of additional equipment, and mounting elements (486).

5. The equipment for detecting of the state of health of the monitored persons according to claim 1, characterized in that
said fixed monitor (349) of heart signals is non-detachably mounted on said auxiliary equipment (64) and comprises said block (1061) of basic units of said monitor, said block (1066) of supplementary units, and said block (21) of pieces of additional equipment.

6. The equipment for detecting of the state of health of the monitored persons according to claim 1, characterized in that
said built in monitor (349) of heart signals is built in said controlling and displaying module (358), or in said controlling and displaying operative module (357), and it comprises said block (1061) of basic units of said monitor, said block (1066) of supplementary units, and said block (21) of pieces of additional equipment.

7. The equipment for detecting of the state of health of the monitored persons according to claim 6, characterized in that
said block (21) of pieces of additional equipment is exchangeable for another one, which is equipped optionally according to requirements, namely for at least one independently operating microprocessor unit (1068), which unit is adapted for processing of applications and/or operation systems.

8. The equipment for detecting of the state of health of the monitored persons according to claim 1 characterized in that
said sensing elements (1065) are formed by at least one of the ECG electrodes (143), an external electrode (143′), electrodes (950), external electrodes (957), on the chest applied electrodes (1013), a bottom electrode (1023) for fingers, contact areas (224) placed on said monitor (349) of heart signals, and/or on cooperating unit (121), and/or on said auxiliary equipment (64).
